SEQUENCE LISTING (2 sequences)

SEQ ID NO: 1 - DNA, 62 bp, synthetic construct
  modified_base 51..52: mod_base = OTHER; note = Thymidine-succinyl hexamide CED phosphoramidite
  source 1..62: mol_type = other DNA
agacaatcaaccatttggggtggacagccttgacctctagacttcggcatnntttttttttt

SEQ ID NO: 2 - DNA, 15 bp, synthetic construct
  misc_feature 1..15: note = Description of Artificial Sequence: Synthetic oligonucleotide
  source 1..15: mol_type = other DNA
agatcagtcagtgtc

(19) United States
(12) Patent Application Publication
(10) Pub. No.: US 2026/0208143 A1
(43) Pub. Date: Jul. 23, 2026

(54) ELECTROCHEMICAL POLYNUCLEOTIDE SYNTHESIS

(71) Applicant: Twist Bioscience Corporation, South San Francisco, CA (US)

(72) Inventors: Jeremy LACKEY, Foster City, CA (US); Maryam HABIBIAN, San Bruno, CA (US); YanYou LIN, Fremont, CA (US)

(73) Assignee: Twist Bioscience Corporation, South San Francisco, CA (US)

(21) Appl. No.: 19/451,765

(22) Filed: Jan. 16, 2026

Related U.S. Application Data
(63) Continuation of application No. 17/702,638, filed on Mar. 23, 2022, now abandoned.
(60) Provisional application No. 63/165,661, filed on Mar. 24, 2021.

(51) Int. Cl. B01J 19/00; C12P 19/34; C25B 3/23; C25B 3/25

(52) U.S. Cl. CPC B01J 19/0046; C12P 19/34; C25B 3/23; C25B 3/25; B01J 2219/00623; B01J 2219/00722

(57) ABSTRACT

Provided herein are compositions, devices, systems and methods for generation and use of biomolecule-based information for storage. Further provided are devices comprising addressable electrodes controlling polynucleotide synthesis (deprotection, extension, or cleavage, etc.) Further provided are compositions for low voltage deprotection of polynucleotides.

### CROSS-REFERENCE

This application is a continuation of U.S. patent application Ser. No. 17,702,638, filed Mar. 23, 2022, which claims priority to U.S. Provisional Patent Application No. 63/165,661 filed on Mar. 24, 2021, each of which are incorporated herein by reference in their entirety.

### SEQUENCE LISTING

The instant application contains a Sequence Listing which has been submitted electronically in .XML format and is hereby incorporated by reference in its entirety. Said .XML copy was created on Jan. 14, 2026, is named “Sequence_Listing.xml”, and is 3,728 bytes in size.

### BACKGROUND

Biomolecule based information storage systems, e.g., DNA-based, have a large storage capacity and stability over time. However, there is a need for scalable, automated, highly accurate and highly efficient systems for generating biomolecules for information storage.

### BRIEF SUMMARY

Provided herein are compositions, methods, devices, and systems for electrochemical polynucleotide synthesis.

Provided herein are compositions for electrochemical acid generation comprising: a first redox compound; a second redox compound; an organic salt; and at least one solvent, wherein the redox potential between the first redox compound and the second redox compound is less than 2 volts in the solvent, and wherein application of a voltage to the composition results in electrochemical acid generation. Further provided herein are compositions wherein the first redox compound or the second redox compound independently has the structure:

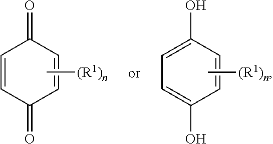

wherein each R¹ is independently hydrogen, halogen, —CN, —OR^{a}, —SR^{a}, —S(═O)R^{b}, —NO₂, —NR^{c}R^{d}, —S(═O)₂R^{d}, —NR^{a}S(═O)₂R^{d}, —S(═O)₂NR^{c}R^{d}, —C(═O)R, —OC(═O)R, —CO₂R^{a}, —OCO₂R^{a}, —C(═O)NR^{c}R^{d}, —OC(═O)NR^{c}R^{d}, —NR^{a}C(═O)NR^{c}R^{d}, —NR^{a}C(═O)R^{b}, —NR^{a}C(═O)OR^{a}, C₁-C₆ alkyl, C₂-C₆ alkenyl, C₂-C₆ alkynyl, C₁-C₆ heteroalkyl, C₃-C₈ cycloalkyl, C₂-C₈ heterocycloalkyl, aryl, or heteroaryl; wherein the alkyl, alkenyl, alkynyl, and heteroalkyl is optionally substituted with one, two, or three of halogen or —OR^{a}; and the cycloalkyl, heterocycloalkyl, aryl, and heteroaryl is optionally substituted with one, two, or three of halogen, C₁-C₆ alkyl, C₁-C₆ haloalkyl, —OR^{a}, or —NR^{c}R^{d}; or two or more R¹ are taken together to form a cycloalkyl, heterocycloalkyl, aryl, or heteroaryl ring; R^{a} is hydrogen, C₁-C₆ alkyl, C₂-C₆ alkenyl, C₂-C₆ alkynyl, C₁-C₆ heteroalkyl, C₃-C₈ cycloalkyl, C₂-C₈ heterocycloalkyl, aryl, or heteroaryl; wherein the alkyl, alkenyl, alkynyl, and heteroalkyl is optionally substituted with one, two, or three of halogen, —OH, —OMe, or —NH₂; and the cycloalkyl, heterocycloalkyl, aryl, and heteroaryl is optionally substituted with one, two, or three of halogen, C₁-C₆ alkyl, C₁-C₆ haloalkyl, —OH, —OMe, or —NH₂; R is C₁-C₆ alkyl, C₂-C₆ alkenyl, C₂-C₆ alkynyl, C₁-C₆ heteroalkyl, C₃-C₈ cycloalkyl, C₂-C₈ heterocycloalkyl, aryl, or heteroaryl; wherein the alkyl, alkenyl, alkynyl, and heteroalkyl is optionally substituted with one, two, or three of halogen, —OH, —OMe, or —NH₂; and the cycloalkyl, heterocycloalkyl, aryl, and heteroaryl is optionally substituted with one, two, or three of halogen, C₁-C₆ alkyl, C₁-C₆ haloalkyl, —OH, —OMe, or —NH₂; each R^{c} and R^{d} is independently hydrogen, C₁-C₆ alkyl, C₂-C₆ alkenyl, C₂-C₆ alkynyl, C₁-C₆ heteroalkyl, C₃-C₈ cycloalkyl, C₂-C₈ heterocycloalkyl, aryl, or heteroaryl; wherein the alkyl, alkenyl, alkynyl, and heteroalkyl is optionally substituted with one, two, or three of halogen, —OH, —OMe, or —NH₂; and the cycloalkyl, heterocycloalkyl, aryl, and heteroaryl is optionally substituted with one, two, or three of halogen, C₁-C₆ alkyl, C₁-C₆ haloalkyl, —OH, —OMe, or —NH₂; or R^{c} and R^{d}, together with the nitrogen atom to which they are attached, form a heterocycloalkyl or heteroaryl; wherein the heterocycloalkyl and heteroaryl is optionally substituted with one, two, or three of halogen, C₁-C₆ alkyl, C₁-C₆ haloalkyl, —OH, —OMe, or —NH₂; and n is 0-4. Further provided herein are compositions wherein the first redox compound has the structure:

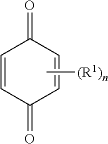

and the second redox compound has the structure:

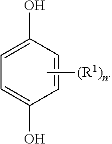

Further provided herein are compositions wherein the first redox compound has the structure:

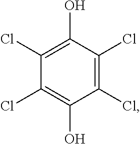 [SMILES: Oc1c(Cl)c(Cl)c(O)c(Cl)c1Cl]

and the second redox compound has the structure

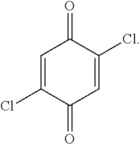

Further provided herein are compositions wherein the first redox compound has the structure:

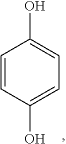

and the second redox compound has the structure

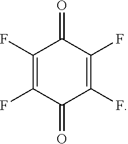 [SMILES: O=C1C(F)=C(F)C(=O)C(F)=C1F]

Further provided herein are compositions wherein the first redox compound has the structure:

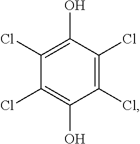 [SMILES: Oc1c(Cl)c(Cl)c(O)c(Cl)c1Cl]

and the second redox compound has the structure

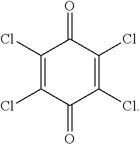 [SMILES: O=C1C(Cl)=C(Cl)C(=O)C(Cl)=C1Cl]

Further provided herein are compositions wherein the first redox compound has the structure:

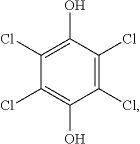 [SMILES: Oc1c(Cl)c(Cl)c(O)c(Cl)c1Cl]

and the second redox compound has the structure

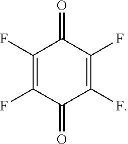 [SMILES: O=C1C(F)=C(F)C(=O)C(F)=C1F]

Further provided herein are compositions wherein the organic salt comprises a tetraalkylammonium cation. Further provided herein are compositions wherein the organic salt comprises a hexafluorophosphate anion. Further provided herein are compositions wherein the organic salt is tetrabutylammonium hexafluorophosphate. Further provided herein are compositions wherein the solvent is acetonitrile, methanol, ethanol, dichloromethane, chloroform, 1,2-dichloromethane, dimethylformamide, ethylene glycol, propylene carbonate, or a mixture thereof. Further provided herein are compositions wherein the concentration of the first redox compound and the concentration of the second redox compound is 0.1-2M. Further provided herein are compositions wherein the concentration of the first redox compound and the concentration of the second redox compound is 0.1-0.5M. Further provided herein are compositions wherein the concentration of the organic salt is 10-50 mM. Further provided herein are compositions wherein the composition does not comprise an amine base. Further provided herein are compositions wherein the amine base is a non-nucleophilic base. Further provided herein are compositions wherein the amine base is 2,6-lutidine, DIPEA, DBU, or pyridine.

Provided herein are methods for polynucleotide synthesis comprising: (a) contacting a nucleoside attached to a solid support with a protected nucleoside, wherein the protected nucleoside is configured to form a covalent bond with the nucleoside to generate a protected polynucleotide; (b) contacting the protected polynucleotide with a composition provided herein, and (c) applying a voltage to a solvent in fluid communication with the protected polynucleotide, wherein the voltage results in deprotection of the terminal nucleoside of the protected polynucleotide. Further provided herein are methods wherein the voltage is less than 2 volts. Further provided herein are methods wherein the voltage is 0.1-2 volts. Further provided herein are methods wherein the voltage is applied for 0.001-5000 seconds. Further provided herein are methods wherein the voltage is applied for 0.001-5 seconds. Further provided herein are methods wherein the voltage is applied in one or more pulses. Further provided herein are methods wherein the time between pulses is 50-500 milliseconds. Further provided herein are methods wherein the protected polynucleotide comprises an acid-cleavable protecting group. Further provided herein are methods wherein the voltage generates acid. Further provided herein are methods wherein the protected polynucleotide is 25-500 bases in length.

Provided herein are devices for polynucleotide synthesis comprising: a surface comprising a plurality of loci configured for polynucleotide synthesis, wherein the composition described herein is in contact with one or more loci; and a plurality of vias and/or routing configured for addressable control of the plurality of loci, wherein the area of each loci is 50-500 nm. Further provided herein are devices wherein the loci comprises a pitch distance of no more than 1000 nm. Further provided herein are devices wherein the loci comprises a pitch distance of no more than 500 nm. Further provided herein are devices wherein the loci comprises a pitch distance of no more than 250 nm. Further provided herein are devices wherein the loci comprises a pitch distance of no more than 100 nm. Further provided herein are devices wherein the device comprises at least 10 loci per square micron. Further provided herein are devices wherein the device comprises at least 5 loci per square micron. Further provided herein are devices wherein the device is integrated into a CMOS. Further provided herein are devices wherein the device further comprises a fluidics interface.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a cyclic voltammogram for different pairs of redox compounds.

FIG. 2A illustrates a cross-section view of a high-density device for polynucleotide synthesis. Two exemplary addressable device arrays are shown for clarity only.

FIG. 2B illustrates a top view of a high-density device for polynucleotide synthesis. Nine exemplary addressable device arrays are shown for clarity only.

FIG. 2C illustrates a top view of a high-density device for polynucleotide synthesis. Four exemplary addressable device arrays are shown for clarity only.

FIG. 2D illustrates a cross-section view of a high-density device for polynucleotide synthesis. Two exemplary device arrays are shown for clarity only.

FIG. 2E illustrates a top view of a high-density device for polynucleotide synthesis. Sixteen exemplary addressable device arrays are shown for clarity only.

FIG. 2F illustrates a cross-section view of a high-density device for polynucleotide synthesis, comprising a device layer for polynucleotide synthesis in communication with a CMOS chip.

FIG. 3A depicts a device for polynucleotide synthesis which is configured for electrochemical deblocking. Lighter regions indicate loci which were selectively deblocked using electrochemical acid generation and subsequently coupled to a fluorescent nucleoside.

FIG. 3B depicts synthesis of a polynucleotide 15-mer using electrochemical deblocking.

FIG. 3B discloses SEQ ID NO: 2.

FIG. 4A illustrates an example of a computer system.

FIG. 4B is a block diagram illustrating architecture of a computer system.

FIG. 4C is a diagram demonstrating a network configured to incorporate a plurality of computer systems, a plurality of cell phones and personal data assistants, and Network Attached Storage (NAS).

FIG. 4D is a block diagram of a multiprocessor computer system using a shared virtual address memory space.

FIG. 5A is a front side of an example of a solid support array. Such arrays in some instances may comprise thousands or millions of polynucleotide synthesis devices as described herein.

FIG. 5B is a back side of an example of a solid support array.

FIG. 5C is a schema of solid support comprising an active area and fluidics interface.

FIG. 5D is an example of rack-style instrument. Such instruments may comprise hundreds or thousands of solid support arrays.

### DETAILED DESCRIPTION OF THE INVENTION

There is a need for larger capacity storage systems as the amount of information generated and stored is increasing exponentially. A biomolecule such as a DNA molecule provides a suitable host for information storage in-part due to its stability over time and capacity for four bit information coding, as opposed to traditional binary information coding. Provided herein are methods to increase DNA synthesis throughput through increased sequence density, increased step efficiency, and decreased turn-around time using electrochemical control of the polynucleotide deblocking step.

### Definitions

Unless defined otherwise, all technical and scientific terms used herein have the same meaning as is commonly understood by one of ordinary skill in the art to which these inventions belong.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

As used herein, the terms “preselected sequence”, “predefined sequence” or “predetermined sequence” are used interchangeably. The terms mean that the sequence of the polymer is known and chosen before synthesis or assembly of the polymer. In particular, various aspects of the invention are described herein primarily with regard to the preparation of nucleic acids molecules, the sequence of the polynucleotide being known and chosen before the synthesis or assembly of the nucleic acid molecules.

Provided herein are methods and compositions for production of synthetic (i.e. de novo synthesized or chemically synthesized) polynucleotides. Polynucleotides may also be referred to as oligonucleotides or oligos. Polynucleotide sequences described herein may be, unless stated otherwise, comprise DNA or RNA.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₈ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2)π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R—N(R^{a})C(O)R^{a}, —R—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” or “cycloalkyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R—N(R^{a})C(O)OR^{a}, —R^{b}—N(R^{a})C(O)R^{a}, —R—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical are optionally substituted as defined above.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” or “heterocycloalkyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R—N(R^{a})C(O)OR^{a}, —R^{b}—N(R^{a})C(O)R^{a}, —R—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R—N(R^{a})C(O)OR^{a}, —R—N(R^{a})C(O)R^{a}, —R—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)—. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

### Nucleic Acid Synthesis

Provided herein are devices, methods, compositions, and systems for nucleic acid storage and synthesis on solid supports. In some instances, solid supports comprise surfaces. Further provided herein are pluralities of devices which are combined to form larger arrays or chips. Further provided herein are devices and methods which are configured for electrochemical deprotection or deblocking during polynucleotide synthesis. Further provided herein are methods which minimize voltages required for deblocking. Further provided herein are combinations of redox-active molecules for electrochemical acid generation. Further provided herein are combinations of redox-active molecules for voltage-controlled deblocking.

Conventional methods of electrochemical acid generation often require voltages in excess of those tolerable by high-density transistor devices (e.g., CMOS). Excess voltages in some instances give rise to unstable currents which reduce the fidelity of deblocking during polynucleotide synthesis. In some instances, methods described herein are configured to operate at voltages less than 2 volts. In some instances, methods described herein are configured for voltages of no more than 2.00, 1.95, 1.9, 1.85, 1.80, 1.75, 1.70, 1.65, 1.60, or no more than 1.50 volts. In some instances, methods described herein are configured for voltages of 0.1-2, 0.1-1.5, 1-1.9, 1-1.8, 1-1.7, 1-1.6 or 1-1.5 volts. In some instances, compositions described herein allow for reduced concentrations of redox compounds relative to previous methods. In some instances, compositions described herein allow for reduced concentrations of additives, such as reduced or eliminate concentrations of bases. In some instances, compositions described herein allow for reduced concentrations of additives, such as reduced or eliminate concentrations of amine bases, (e.g., 2,6-lutidine).

Described herein are redox compositions which facilitate electrochemical acid generation. In some instances, a composition comprises one or more redox compounds. In some instances, a composition comprises a first redox compound and a second redox compound. In some instances, the first redox compound is a quinone or substituted derivative thereof. In some instances, the second redox compound is a hydroquinone or substituted derivative thereof. In some instances, a redox compound has the structure:

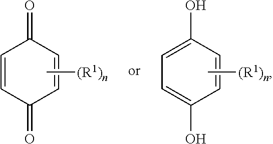

wherein
each R¹ is independently halogen, —CN, —OR^{a}, —SR^{a}, —S(═O)R, —NO₂, —NR^{c}R^{d}, —S(═O)₂R^{d}, —NR^{a}S(═O)₂R^{d}, —S(═O)₂NR^{c}R^{d}, —C(═O)R, —OC(═O)R, —CO₂R^{a}, —OCO₂R^{a}, —C(═O)NR^{c}R^{d}, —OC(═O)NR^{c}R^{d}, —NR^{a}C(═O)NR^{c}R^{d}, —NR^{a}C(═O)R^{b}, —NR^{a}C(═O)OR^{a}, C₁-C₆ alkyl, C₂-C₆ alkenyl, C₂-C₆ alkynyl, C₁-C₆ heteroalkyl, C₃-C₈ cycloalkyl, C₂-C₈ heterocycloalkyl, aryl, or heteroaryl; wherein the alkyl, alkenyl, alkynyl, and heteroalkyl is optionally substituted with one, two, or three of halogen or —OR^{a}; and the cycloalkyl, heterocycloalkyl, aryl, and heteroaryl is optionally substituted with one, two, or three of halogen, C₁-C₆ alkyl, C₁-C₆ haloalkyl, —OR^{a}, or —NR^{c}R^{d}; or two or more R¹ are taken together to form a cycloalkyl, heterocycloalkyl, aryl, or heteroaryl ring;
R^{a} is hydrogen, C₁-C₆ alkyl, C₂-C₆ alkenyl, C₂-C₆ alkynyl, C₁-C₆ heteroalkyl, C₃-C₈ cycloalkyl, C₂-C₅ heterocycloalkyl, aryl, or heteroaryl; wherein the alkyl, alkenyl, alkynyl, and heteroalkyl is optionally substituted with one, two, or three of halogen, —OH, —OMe, or —NH₂; and the cycloalkyl, heterocycloalkyl, aryl, and heteroaryl is optionally substituted with one, two, or three of halogen, C₁-C₆ alkyl, C₁-C₆ haloalkyl, —OH, —OMe, or —NH₂;
R^{b} is C₁-C₆ alkyl, C₂-C₆ alkenyl, C₂-C₆ alkynyl, C₁-C₆ heteroalkyl, C₃-C₈ cycloalkyl, C₂-C₈ heterocycloalkyl, aryl, or heteroaryl; wherein the alkyl, alkenyl, alkynyl, and heteroalkyl is optionally substituted with one, two, or three of halogen, —OH, —OMe, or —NH₂; and the cycloalkyl, heterocycloalkyl, aryl, and heteroaryl is optionally substituted with one, two, or three of halogen, C₁-C₆ alkyl, C₁-C₆ haloalkyl, —OH, —OMe, or —NH₂;
each R^{c} and R^{d} is independently hydrogen, C₁-C₆ alkyl, C₂-C₆ alkenyl, C₂-C₆ alkynyl, C₁-C₆ heteroalkyl, C₃-C₈ cycloalkyl, C₂-C₈ heterocycloalkyl, aryl, or heteroaryl; wherein the alkyl, alkenyl, alkynyl, and heteroalkyl is optionally substituted with one, two, or three of halogen, —OH, —OMe, or —NH₂; and the cycloalkyl, heterocycloalkyl, aryl, and heteroaryl is optionally substituted with one, two, or three of halogen, C₁-C₆ alkyl, C₁-C₆ haloalkyl, —OH, —OMe, or —NH₂;
or R^{c} and R^{d}, together with the nitrogen atom to which they are attached, form a heterocycloalkyl or heteroaryl; wherein the heterocycloalkyl and heteroaryl is optionally substituted with one, two, or three of halogen, C₁-C₆ alkyl, C₁-C₆ haloalkyl, —OH, —OMe, or —NH₂; and
n is 0-4.

In some instances, n is 0. In some instances, n is 1. In some instances, n is 2. In some instances, n is 3. In some instances, n is 4. In some instances, R¹ is independently selected from —CN, hydrogen, C₁-C₆ alkyl, halo, —OH, or —NH₂. In some instances, R¹ is independently C₁, F, Br, or I. In some instances, at least one R¹ is not hydrogen. In some instances, two or more R¹ are taken together to form a cycloalkyl, heterocycloalkyl, aryl, or heteroaryl ring. In some instances, two or more R¹ are taken together to form an aryl or heteroaryl ring. In some instances, a redox compound has the structure:

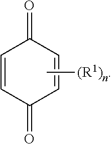

In some instances, a redox compound has the structure:

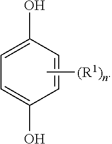

In some instances, a redox compound has the structure:

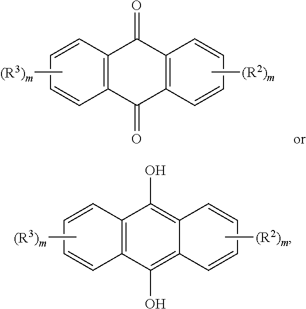

wherein
each R² or R³ is independently hydrogen, halogen, —CN, —OR^{a}, —SR^{a}, —S(═O)R, —NO₂, —NR^{c}R^{d}, —S(═O)₂R^{d}, —NR^{a}S(═O)₂R^{d}, —S(═O)₂NR^{c}R^{d}, —C(═O)R, —OC(═O)R, —CO₂R^{a}, —OCO₂R^{a}, —C(═O)NR^{c}R^{d}, —OC(═O)NR^{c}R^{d}, —NR^{a}C(═O)NR^{c}R^{d}, —NR^{a}C(═O)R^{b}, —NR^{a}C(═O)OR^{a}, C₁-C₆ alkyl, C₂-C₆ alkenyl, C₂-C₆ alkynyl, C₁-C₆ heteroalkyl, C₃-C₈ cycloalkyl, C₂-C₈ heterocycloalkyl, aryl, or heteroaryl; wherein the alkyl, alkenyl, alkynyl, and heteroalkyl is optionally substituted with one, two, or three of halogen or —OR^{a}; and the cycloalkyl, heterocycloalkyl, aryl, and heteroaryl is optionally substituted with one, two, or three of halogen, C₁-C₆ alkyl, C₁-C₆ haloalkyl, —OR^{a}, or —NR^{c}R^{d}; or two or more R² or R³ are taken together to form a cycloalkyl, heterocycloalkyl, aryl, or heteroaryl ring;
R^{a} is hydrogen, C₁-C₆ alkyl, C₂-C₆ alkenyl, C₂-C₆ alkynyl, C₁-C₆ heteroalkyl, C₃-C₈ cycloalkyl, C₂-C₅ heterocycloalkyl, aryl, or heteroaryl; wherein the alkyl, alkenyl, alkynyl, and heteroalkyl is optionally substituted with one, two, or three of halogen, —OH, —OMe, or —NH₂; and the cycloalkyl, heterocycloalkyl, aryl, and heteroaryl is optionally substituted with one, two, or three of halogen, C₁-C₆ alkyl, C₁-C₆ haloalkyl, —OH, —OMe, or —NH₂;
R^{b} is C₁-C₆ alkyl, C₂-C₆ alkenyl, C₂-C₆ alkynyl, C₁-C₆ heteroalkyl, C₃-C₈ cycloalkyl, C₂-C₈ heterocycloalkyl, aryl, or heteroaryl; wherein the alkyl, alkenyl, alkynyl, and heteroalkyl is optionally substituted with one, two, or three of halogen, —OH, —OMe, or —NH₂; and the cycloalkyl, heterocycloalkyl, aryl, and heteroaryl is optionally substituted with one, two, or three of halogen, C₁-C₆ alkyl, C₁-C₆ haloalkyl, —OH, —OMe, or —NH₂;
each R^{c} and R^{d} is independently hydrogen, C₁-C₆ alkyl, C₂-C₆ alkenyl, C₂-C₆ alkynyl, C₁-C₆ heteroalkyl, C₃-C₈ cycloalkyl, C₂-C₈ heterocycloalkyl, aryl, or heteroaryl; wherein the alkyl, alkenyl, alkynyl, and heteroalkyl is optionally substituted with one, two, or three of halogen, —OH, —OMe, or —NH₂; and the cycloalkyl, heterocycloalkyl, aryl, and heteroaryl is optionally substituted with one, two, or three of halogen, C₁-C₆ alkyl, C₁-C₆ haloalkyl, —OH, —OMe, or —NH₂;
or R^{c} and R^{d}, together with the nitrogen atom to which they are attached, form a heterocycloalkyl or heteroaryl; wherein the heterocycloalkyl and heteroaryl is optionally substituted with one, two, or three of halogen, C₁-C₆ alkyl, C₁-C₆ haloalkyl, —OH, —OMe, or —NH₂; and
m is 0-8.

In some instances, m is 0. In some instances, m is 1. In some instances, m is 2. In some instances, m is 3. In some instances, m is 4. In some instances, at least one R² is not hydrogen. In some instances, at least one R³ is not hydrogen. In some instances, R² or R³ is independently selected from —CN, hydrogen, C₁-C₆ alkyl, halo, —OH, or —NH₂. In some instances, R² or R³ is independently C₁, F, Br, or I. In some instances, a redox compound is selected from a compound of Table 1.

TABLE 1
Redox Compounds

| # | Oxidized Structure | # | Reduced Structure |
|---|---|---|---|
| 1A | 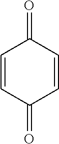 | 1B | 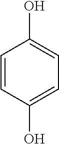 |
| 2A | 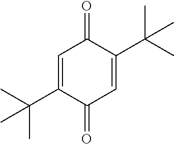 [SMILES: CC(C)(C)C1=CC(=O)C(C(C)(C)C)=CC1=O] | 2B | 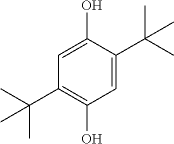 [SMILES: CC(C)(C)c1cc(O)c(C(C)(C)C)cc1O] |
| 3A | 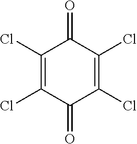 [SMILES: O=C1C(Cl)=C(Cl)C(=O)C(Cl)=C1Cl] | 3B | 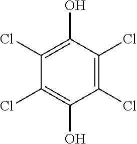 [SMILES: Oc1c(Cl)c(Cl)c(O)c(Cl)c1Cl] |
| 4A | 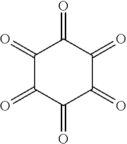 [SMILES: O=C1C(=O)C(=O)C(=O)C(=O)C1=O] | 4B | 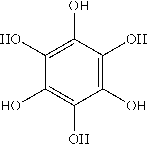 [SMILES: Oc1c(O)c(O)c(O)c(O)c1O] |
| 5A | 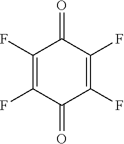 [SMILES: O=C1C(F)=C(F)C(=O)C(F)=C1F] | 5B | 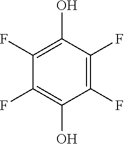 [SMILES: Oc1c(F)c(F)c(O)c(F)c1F] |
| 6A | 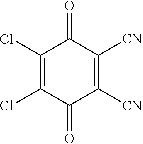 [SMILES: N#CC1=C(C#N)C(=O)C(Cl)=C(Cl)C1=O] | 6B | 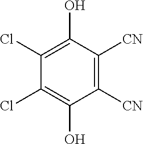 [SMILES: N#Cc1c(O)c(Cl)c(Cl)c(O)c1C#N] |
| 7A | 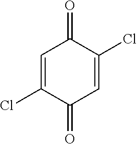 | 7B | 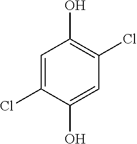 |
| 8A | 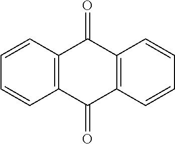 [SMILES: O=C1c2ccccc2C(=O)c2ccccc21] | 8B | 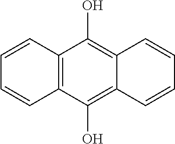 [SMILES: Oc1c2ccccc2c(O)c2ccccc12] |
| 9A | 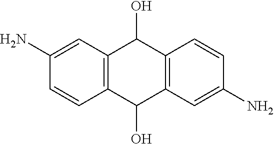 [SMILES: Nc1ccc2c(c1)C(O)c1ccc(N)cc1C2O] | 9B | 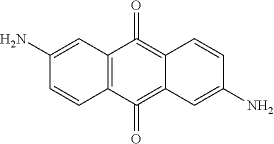 |

Redox compositions may comprise a first redox compound and a second redox compound (e.g., redox “pair”). In some instances, a redox pair is selected from pairs E1-E43 in Table 3. In some instances, a first redox compound is selected the “B” column of Table 1. In some instances, a second redox compound is selected the “A” column of Table 1. In some instances, the first redox compound has the structure:

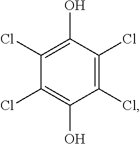 [SMILES: Oc1c(Cl)c(Cl)c(O)c(Cl)c1Cl]

and the second redox compound has the structure

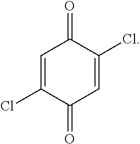

In some instances, the first redox compound has the structure:

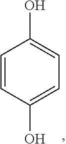

and the second redox compound has the structure

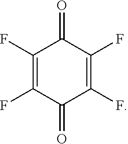 [SMILES: O=C1C(F)=C(F)C(=O)C(F)=C1F]

In some instances, the first redox compound has the structure:

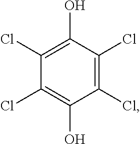 [SMILES: Oc1c(Cl)c(Cl)c(O)c(Cl)c1Cl]

and the second redox compound has the structure

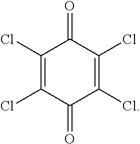 [SMILES: O=C1C(Cl)=C(Cl)C(=O)C(Cl)=C1Cl]

In some instances, first redox compound has the structure:

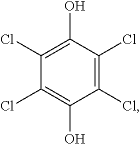 [SMILES: Oc1c(Cl)c(Cl)c(O)c(Cl)c1Cl]

and the second redox compound has the structure

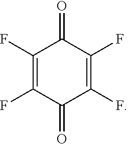 [SMILES: O=C1C(F)=C(F)C(=O)C(F)=C1F]

In some instances, the first redox compound and the second redox compound are selected such that a redox potential is less than 2 volts. In some instance, a first redox compound is present in a reduced state. In some instance, a second redox compound is present in an oxidized state. In some instances, redox compositions described herein further comprise a plurality of polynucleotides. In some instances, at least some of the polynucleotides comprise acid-sensitive deblocking groups.

The concentration of a redox compound may be varied to achieve a desired redox potential. In some instances, a redox compound is present at 0.1-1M. In some instances, a redox compound is present at 0.1-0.5, 0.1-0.9, 0.3-0.7, 0.4-0.6, 0.4-1, 0.5-1, or 0.6-1M. In some instances, a redox compound is present at about 0.1, 0.2, 0.3, 0.4, 0.5, 0.6, 0.7, 0.8, 0.9, or about 1M. In some instances, a redox compound is present at no more than 0.1, 0.2, 0.3, 0.4, 0.5, 0.6, 0.7, 0.8, 0.9, or no more than 1M. In some instances, a first redox compound is present at 0.1-0.5, 0.1-0.9, 0.3-0.7, 0.4-0.6, 0.4-1, 0.5-1, or 0.6-1M. In some instances, a first redox compound is present at 0.1, 0.2, 0.3, 0.4, 0.5, 0.6, 0.7, 0.8, 0.9, or about 1M. In some instances, a second redox compound is present at 0.1-0.5, 0.1-0.9, 0.3-0.7, 0.4-0.6, 0.4-1, 0.5-1, or 0.6-1M. In some instances, a second redox compound is present at 0.1, 0.2, 0.3, 0.4, 0.5, 0.6, 0.7, 0.8, 0.9, or about 1M.

The redox compositions described herein may comprise additional components. In some instances, a redox composition comprises a salt. In some instances, the salt is an organic salt. In some instances, the organic salt is a phase-transfer catalyst. In some instances, the organic salt comprises a tetra alkyl ammonium cation. In some instances, the organic salt comprises an tetrabutylammonium cation. In some instances, the organic salt comprises an tetramethylammonium cation. In some instances, the organic salt comprises an hexafluorophosphate cation. In some instances, the organic salt comprises hexafluorophosphate, tetrafluoroborate, tetraphenylborate, hexafluorophosphate, perchlorate, tetrachloroferrate, hexafluoroarsenate, hexafluoroantimonate, pentafluorohy-droxyantimonate, hexachloroantimonate, tetrakispentafluorophenylborate, tetrakis-(pentafluoromethylphenyl) borate,bi(trifluoromethylsulphonyl)amides or tris(trifluoromethylsulphonyl)methides. In some instances, the concentration of a salt (e.g., organic salt) is 10-50 mM. In some instances, the concentration of a salt (e.g., organic salt) is 10-50 mM, 5-100 mM, 15-75 mM, 25-100 mM, 25-50 mM, 15-25 mM, or 15-50 mM. In some instances, the concentration of a salt (e.g., organic salt) is about 5, 10, 15, 20, 25, 30, 35, 40, 45, 50, 55, 60, or about 65 mM. In some instances, the concentration of a salt (e.g., organic salt) is no more than 5, 10, 15, 20, 25, 30, 35, 40, 45, 50, 55, 60, or no more than 65 mM.

The redox compositions described herein may comprise at least one solvent. In some instances, the solvent is an organic solvent. In some instances, the solvent comprises hydrocarbons (e.g., hexane, decane, benzene, toluene, xylene, isomers thereof, and the like), ethers (e.g., THF, diethyl ether, methyl t-butyl ether, and the like), esters (e.g., methyl acetate, ethyl acetate, tert-butylacetate, etc.), lactones, ketones (e.g., acetone, methyl ethyl ketone, cyclopentanone, and the like), alcohols (e.g., ethanol, butanol, isopropanol, and the like), amides (e.g., DMF, N-methylpyrrolidinone, or other amides), ureas, carbonates (e.g., diethylcarbonate, or other carbonate), carbamates, aldehydes, amines, cyanates, isocyanates, sulfoxides, sulfones, aromatics, heteroaromatics, thiols, phosphoramides, nitriles (e.g., acetonitrile), alkynes, alkenes, alkanes, halogenated solvents (e.g., tetrachloromethane, dichloromethane, chloroform, or other halogenated solvent), silanes, perfluorocarbons (C₂-C₁₈ perfluorinated branched or straight alkanes such as perfluorohexane, perfluoroheptane, perfluorodecane, perfluoro aromatics such as perfluorobenzene, or other perfluorocarbon), supercritical fluids, ionic liquids, compressed gases, and the like. In some instances, solvents comprise a nitrile, such as acetonitrile. In some instances, solvents comprise an alcohol, such as methanol or ethanol. In some instances, solvents comprise a halogenated solvent, such as dichloromethane, chloroform, or 1,2-dichloromethane. Any of the solvents described herein may be present as a mixture.

Compositions described herein may be substantially free from bases. In some instances, the base is a non-nucleophilic base. In some instances, base is an amine base. Non-limited examples of amine bases include 2,6-lutidine, diisopropylethylamine, 1,8-diazabicyclo[5.4.0]undec-7-ene (DBU), 1,5-Diazabicyclo(4.3.0)non-5-ene (DBN), 2,6-Di-tert-butylpyridine, pyridine, or other amine base. In some instances the base is a phosphazene base, such as t-Bu-P₄.

Redox compositions described herein may result in higher levels of spatial control over deblocking. In some instances, neighboring (e.g., adjacent) loci for polynucleotide synthesis which are not subjected to deblocking voltages are not deblocked. In some instances, adjacent loci are deblocked less than 1%, 0.5%, 0.2%, 0.1%, 0.05%, 0.01%, 0.005%, 0.001% or less than 0.0001%.

### Synthesis Devices

Provided herein are devices for polynucleotide synthesis. Such devices in some instances comprise a solid support comprising a plurality of features (or loci) for polynucleotide synthesis. Such devices may comprise conductive elements or electrodes. Such electrodes may function as anodes or cathodes. Polynucleotides comprise a protecting or blocking group bound to a terminal base during at least one synthesis cycle. Application of a voltage through electrodes during a synthesis step in some instances generates a deprotection reagent (such as an acid or other deprotection reagent) which deblocks polynucleotides (removes the protection group). The length of time the voltage is applied, number of times it applied, and other variables have a significant effect on the extent of deprotection, speed of deprotection, and reduction in unwanted side reactions caused by excess deblocking reagents. The geometry of the device's surface and electrodes also may influence the efficiency of the deblocking step.

Provided herein are devices for polynucleotide synthesis comprising layers of materials. Such devices may comprise any number of layers of materials comprising conductors, semiconductors, or insulative materials. Traditional devices comprise a base layer, conducting materials (one or more conducting layers configured for use as an electrode; conducting materials may be buried in the base layer or above the base layer), and a porous growth layer surface. In some instances, conductive layer is in electrical contact with layer. Each of such layers may be individually patterned to generate features for polynucleotide synthesis such as pores, holes, wells, channels, or other shape. Various layers of such devices are in some instances combined to form addressable solid supports. Layers or surfaces of such devices may be in fluid communication with solvents, solutes, or other reagents used during polynucleotide synthesis. Further described herein are devices comprising a plurality of surfaces. In some instances, surfaces comprise features for polynucleotides synthesis in proximity to conducting materials. In some instances, devices described herein comprise 1, 2, 5, 10, 50, 100, or even thousands of surfaces per device. In some instances, a voltage is applied to one or more layers of a device described herein to facilitate polynucleotide synthesis. In some instances, a voltage is applied to one or more layers of a device described herein to facilitate a step in polynucleotide synthesis, such as deblocking. Different layers on different surfaces of different devices are often energized with a voltage at varying times or with varying voltages. For example, a positive voltage is applied to a first layer, and a negative voltage is applied to a second layer of the same or a different device. In some instances, one or more layers on different devices are energized, while others are disconnected from a ground. In some instances, base layers comprise additional circuitry, such as complementary metal-oxide-semiconductors (CMOS) devices. In some instances, various layers of one or more devices are connected laterally via routing, and/or vertically with vias. In some instances, various layers of one or more devices are connected laterally via routing, and/or vertically with vias to a CMOS layer. In some instances, various layers of one or more devices are connected to a CMOS device via wire bonds, pogo pin contacts, or through Si Vias (TSV).

A first device provided herein comprises a base layer, and a patterned top layer. In some instances, the top layer comprises a conducting material (conducting layer). In some instances, a polynucleotide synthesis surface is formed on the solvent-exposed surface of the base layer. Such a device provides fluid communication between the polynucleotide synthesis surface and the top layer.

A second device provided herein comprises a base layer, a buried shield electrode, and a patterned top layer. In some instances, the top layers and comprise a conducting material. In some instances, devices comprise a conducting layer present in the base layer. In some instances, a polynucleotide synthesis surface is formed by pores in the top layer. Such a device provides fluid communication between the polynucleotide synthesis surface and the top layer. In some instances, the buried shield electrode does not contact the synthesis surface or top layer. In some instances, voltage is passed through the shield electrode to influence the flow of ions in a solvent which contacts the synthesis surface. In some instances a different voltage is applied to the shield electrode compared to the voltage applied to the top layer. In some instances, a voltage applied to the shield electrode is synchronized with an adjacent or proximate conducting layer. In some instances, the time between a voltage applied to the shield electrode and the proximate anode is no more than 0.001 microseconds, 0.1, 0.2, 0.5, 0.8, 1.0, 1.2, 1.5, 1.8, 2, 5, 8, 10, 12, 15, 20, 50, 80, or no more than 100 microseconds. In some instances, the time between a voltage applied to the shield electrode and the proximate anode is no more than 0.001 seconds, 0.1, 0.2, 0.5, 0.8, 1.0, 1.2, 1.5, 1.8, 2, 5, 8, 10, 12, 15, 20, 50, 80, or no more than 100 seconds. In some instances, the time between a voltage applied to the shield electrode and the proximate anode is no more than 0.1 microsecond, 0.2, 0.5, 0.8, 1.0, 1.2, 1.5, 1.8, 2, 5, 8, 10, 12, 15, 20, 50, 80, or no more than 100 microseconds. In some instances, the time between a voltage applied to the shield electrode and the proximate anode is about 0.1 microsecond, 0.2, 0.5, 0.8, 1.0, 1.2, 1.5, 1.8, 2, 5, 8, 10, 12, 15, 20, 50, 80, or about 100 microseconds. In some instances, the time between a voltage applied to the shield electrode and the proximate anode is 0.1-1, 0.1-5, 0.1-10, 0.1-100, 0.5-10, 0.5-100, 1-10, 1-50, 1-100, 5-50, 10-100 or 50-100 microseconds.

A third device provided herein comprises a base layer, and an intermediate layer, and a top layer. In some instances, the intermediate layer and layer comprise a conducting material. In some instances, the top layer comprises a polynucleotide synthesis surface. Such a device provides fluid communication between the polynucleotide synthesis surface and the intermediate layer. The polynucleotide synthesis surfaces in some instances are patterned as cylinders, substantially rectangular shapes, channels, or other shape. In some instances, polynucleotide synthesis surfaces are randomly distributed. In some instances, the intermediate layer comprises a thermal oxide. Devices in some instances comprise one or more additional bonding layers between the synthesis surface and the bottom layer.

A fourth device provided herein comprises a base layer, a first intermediate layer, a top layer. In some instances, the first intermediate comprises a polynucleotide synthesis surface. In some instances, the smallest feature dimension is. In some instances, the smallest feature dimension is proportional to the diffusion distance of a reagent generated proximate to a conducting layer.

A fifth device provided herein comprises a base layer, a first intermediate layer, a second intermediate layer, a top layer. In some instances, polynucleotides are synthesized on top layer. The polynucleotide synthesis surfaces in some instances are patterned as cylinders, substantially rectangular shapes, channels, or other shape. In some instances, polynucleotide synthesis surfaces are randomly patterned. In some instances, the smallest feature dimension is. In some instances, a device comprises additional bonding layers. In some instances, the smallest feature dimension is proportional to the diffusion distance of a reagent generated proximate to a conducting layer. In some instances, a device comprises a conductive layer configured for use as a cathode which is above the plane of one or more conductive layers configured for use as an anode (attached to a lower conductive layer). In some instances, the anode is in fluid communication with one or more loci for polynucleotide synthesis. In another configuration, a conductive layer configured for use as a cathode and located in the same plane as one or more conductive layers configured for use as an anode (attached to a lower conductive layer).

A sixth device provided herein comprises a plurality of addressable solid supports, which are in fluid communication with the flow cell area. In some instances, such a device is used to evaluate operational variables of the device. Each surface comprises a plurality of features for polynucleotide synthesis surrounded by a conducting layer. In some instances, a device comprises at least 1, 2, 5, 10, 20, 50, or more than 50 addressable solid supports. In some instances, the surfaces comprise a series of patterned features such as pores or wells for polynucleotide synthesis. The smallest feature dimension in some instances is the diameter of the wells and/or the distance between wells. In some instances, a device comprises at least 1, 2, 5, 10, 20, 50, or more than 50 addressable solid supports. In some instances, the surfaces comprise a series of patterned features such as channels for polynucleotide synthesis. The smallest feature dimension in some instances is the width of the channels and/or the distance between channels. The smallest feature dimension in some instances is the width of the channels and/or the distance between channels. In some instances, surfaces are located on a device such to maximize the available surface area. In some instances, the distance between any two surfaces is 5-1000 microns, 10-500, 50-500, 5-100, 3-10, 3-50, 25-500 or 50-1000 microns. Additional patterns of features are also in some instances used with the devices described herein. In some instances, the pattern of features on a device are random.

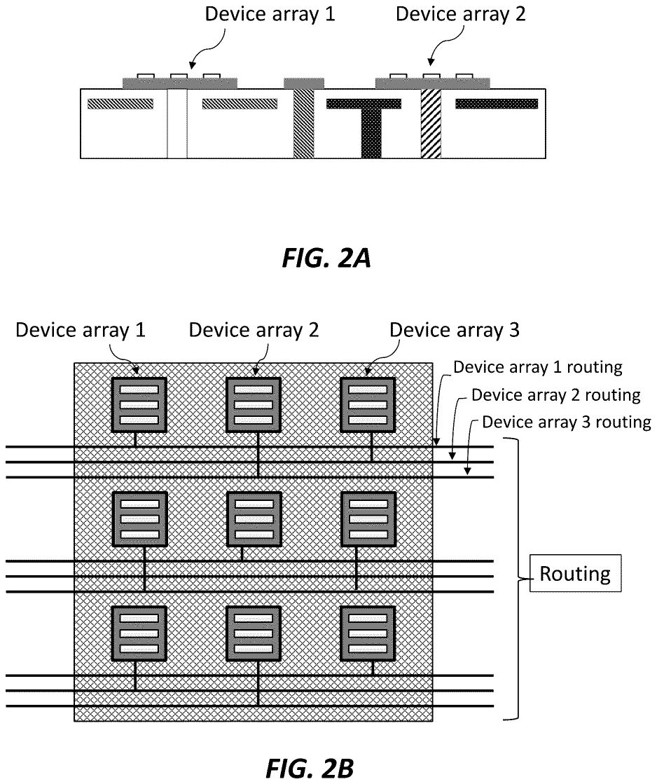

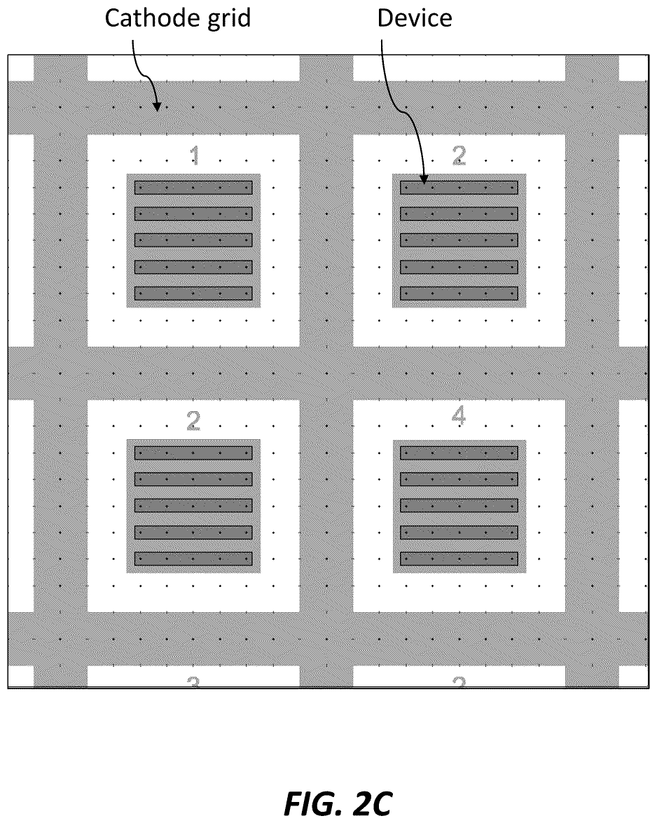

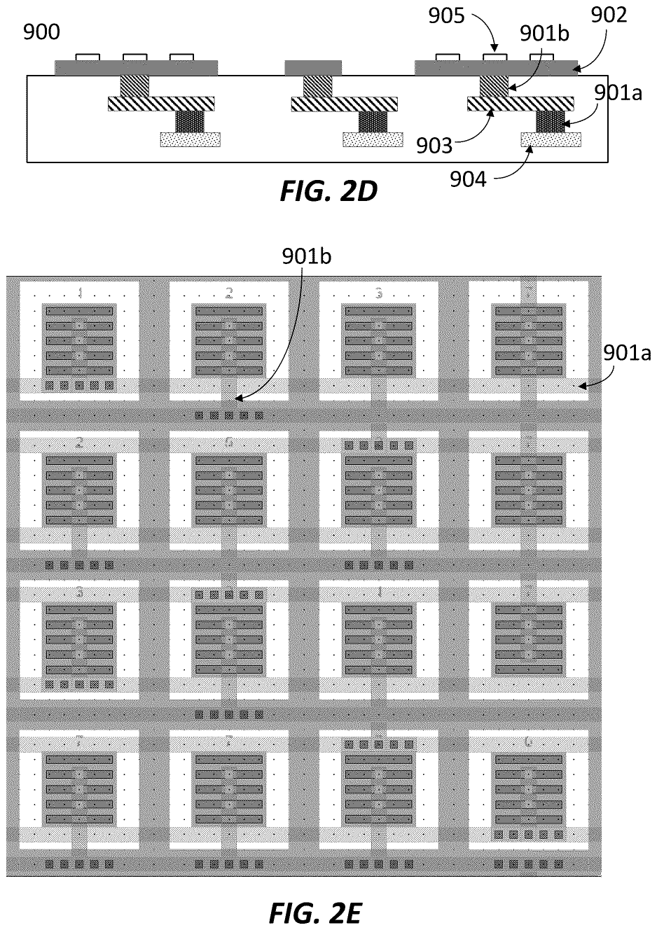

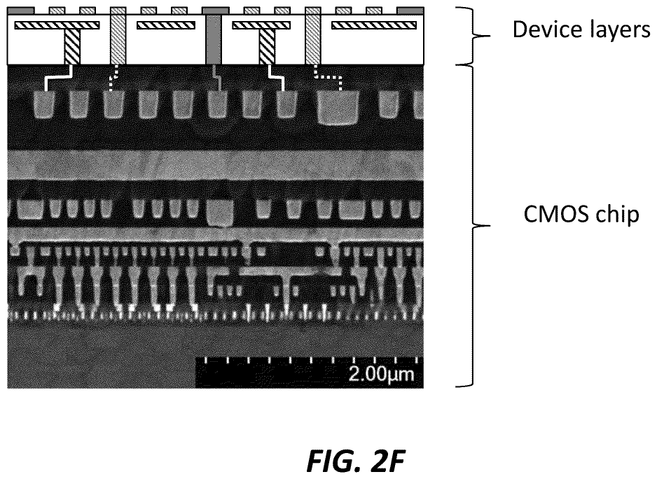

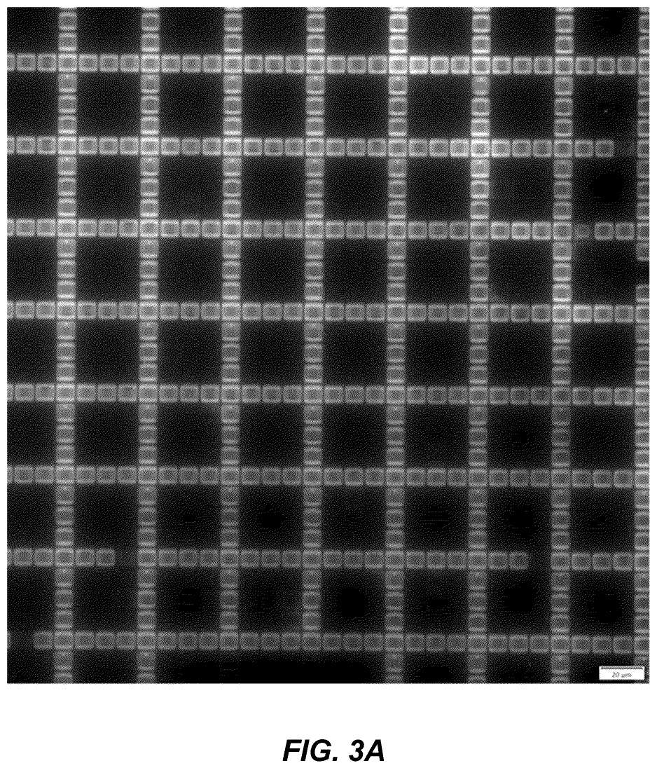

A seventh device described herein comprises a plurality of device arrays (or addressable solid supports), as shown in FIGS. 2A-2E. FIG. 2A shows two such device arrays for clarity, although such devices may comprise any number of device arrays. Nine such device arrays are shown in FIG. 2B, along with routing connections which allow addressable control of individual or groups of device arrays. In some instances, device array 1 is individually addressable from device arrays 2 and 3. Four such device arrays are shown in FIG. 2C for clarity only; any number of devices may be arrayed in this way. The four devices are addressable in groups 1, 2, and 4 as shown for clarity only; the number of addressable groups in some instances is equal to or less than the number of total device arrays. Conductive layers 902 in some instances generate reagents (e.g., acid) for electrochemical deprotection of biomolecules, such as polynucleotides. In some instances, 902 is configured for use as an anode. In some instances, a conducting layer is also configured for use as a cathode as shown in FIG. 2C. A cross section of devices of FIG. 2C or 2E are shown in FIG. 2D. In some instances, polynucleotides are synthesized on oxide layer 905. In some instances, polynucleotides are synthesized on conductive layer 902. Such devices are in some instances addressable by a first layer of routing 901a and a second layer of routing 901b. Such devices may comprise any number of routing layers, for example 1, 2, 3, 4, 5, 10, 20, 50, 100, or more than 100 layers of routing. Routing in different horizontal planes in some instances is connected by one or more vertical interconnect accesses (VIAs), 903 and 904. Such devices may comprise any number of vias, for example 1, 2, 3, 4, 5, 10, 20, 50, 100, 1000, or more than 1000 vias per square micron. The number and size of routing and vias in some instances are proportional to the number of addressable solid supports on a device. A device comprising 16 device arrays (similar to FIG. 2C) is shown in FIG. 2E. Routing 901b is superficial to routing 901a in the device. The sixteen device arrays of FIG. 2E are in some instances addressable as seven groups, but other configurations are also consistent with the devices and methods described herein.

Devices may comprise any number of device arrays. In some instances, devices comprise at least 10, 50, 100, 1000, 10,000, 100,000, or more than 100,000 device arrays in a single device. In some instances, devices comprise about 10, 50, 100, 1000, 10,000, 100,000, or about 100,000 device arrays in a single device. In some instances, devices comprise 10-50, 10-5000, 10-10,000, 100-1000, 100-10,000, 100-100,000, 1000-10,000, or 1000-100,000 device arrays in a single device.

Device arrays may be scaled to any size or dimensions. In some instances, device arrays are about 0.01, 0.02, 0.05, 0.1, 0.2, 0.5, 0.8, 1, 2, 5, 8, or about 10 microns in width. In some instances, device arrays are no more than 0.01, 0.02, 0.05, 0.1, 0.2, 0.5, 0.8, 1, 2, 5, 8, or no more than 10 microns in width. In some instances, device arrays are 0.01-10, 0.1-10, 0.1-1, 0.5-1, 1-10, or 5-30 microns in width. In some instances, device arrays are separated by about 0.01, 0.02, 0.05, 0.1, 0.2, 0.5, 0.8, 1, 2, 5, 8, or about 10 microns. In some instances, device arrays are separated by no more than 0.01, 0.02, 0.05, 0.1, 0.2, 0.5, 0.8, 1, 2, 5, 8, or no more than 10 microns. In some instances, device arrays are separated by 0.01-10, 0.1-10, 0.1-1, 0.5-1, 1-10, or 5-30 microns.

Devices with addressable device arrays may be addressed in different patterns or configurations. In some instances, only specific groups of device arrays are activated simultaneously. In some instances, device arrays are addressed according to. Any number of device arrays may be activated simultaneously. In some instances, about 1%, 2%, 5%, 10%, 20%, 50%, 75%, or about 100% of the device arrays in a device described herein are activated simultaneously. In some instances, no more than 1%, 2%, 5%, 10%, 20%, 50%, 75%, or no more than 99% of the device arrays in a device described herein are activated simultaneously. In some instances, at least 1%, 2%, 5%, 10%, 20%, 50%, 75%, or at least 99% of the device arrays in a device described herein are activated simultaneously.

Devices described herein may be fabricated using numerous methods, such as masking methods. In some instances, a lift-off fabrication method is used. Lift-off methods in some instances comprises addition of a sacrificial layer (e.g., photoresist or “PR”) to a base layer coated with an oxide layer, addition of a conductive layer, and removal of the sacrificial layer. In some instances, a dry-etch fabrication method is used. Dry-etch methods in some instances comprises addition of one or more layers to a base layer, such as an oxide layer, a first intermediate layer (e.g., TiN, or other material), a conductive layer (e.g., platinum), a second intermediate layer (e.g., TiN, or other material), and a sacrificial layer (e.g., photoresist); partial removal of the second intermediate layer to expose the conductive layer; partial removal of the conductive layer to expose the first intermediate layer; partial removal of the first conductive layer to expose the first intermediate layer; and partial removal of the first intermediate layer to expose the oxide layer.

### Electrochemistry

Provided herein are methods of applying voltage to devices with the redox compositions described herein. Such voltages may result in any number of different effects, such as electrochemical reaction with solvents or solutes. In some instances, apply a voltage results in deblocking or removal of protecting groups on molecules attached to a synthesis surface. In some instances molecules are polysaccharides, polynucleotides, polypeptides, or other polymer. In some instances, apply a voltage results in deblocking nucleic acids using the devices described herein. Further provided herein are methods wherein the devices described herein are energized with an electrical voltage. In some instances, the electrical voltage is used to deblock oligonucleotides bound to a solid support or surface. Such deblocking in some instances occurs through direct electrochemical reaction of a blocking group on a polynucleotide, or through the generation of deblocking reagents, such as an acid.

Methods described herein may comprising energizing a device with a voltage (applying a voltage) for a period of time. Applied voltages in some instances form a circuit between a cathode and an anode, leading to current flow through the device, solvent, and/or other components. In some instances, a layer of a device is configured as an anode or cathode. In some instances, a device comprises an anode which is located above the plane of a cathode (“sandwiched”). In some instances, conductive layer is in electrical contact with layer. In some instances, a device comprises an anode which is located in substantially the same plane of the cathode. Application of voltage in some instances is configured to perform a step of polynucleotide synthesis. In some instances, methods comprise application of a voltage to deblock polynucleotides. In some instances application of a voltage generates a deblocking reagent. In some instances, devices comprise conducting layers in fluid communication with a solvent, wherein the solvent comprises reagents which generate a deblocking reagent. In some instances, the deblocking reagent is an acid. In some instances, the acid is H+.

Methods described herein may comprise applying a voltage to one or more devices described herein. In some instances, such voltages result in deprotection of molecules (polynucleotides, polypeptides, polysaccharides, or other polymer) at one or more devices or regions. In some instances, application of a voltage at one or more devices results in deprotection of polynucleotides at one or more devices or regions within one or more devices. In some instances, a device is described as “inactive” if a deprotection reagent (e.g., acid or other reagent) is not generated at or in the vicinity of a device or region of a device. In some instances, a device is described as “active” if a deprotection reagent (e.g., acid or other reagent) is generated at or in the vicinity of a device or region of a device. In some instances, deprotection of polynucleotides occurs at or near one or more active devices, or regions of one or more active devices. In some instances, both active and inactive devices are energized with voltages. In some instances, voltage is applied to inactive devices in levels which are insufficient to generate a deprotection reagent. In some instances, deprotection comprises application of one or more voltages (or voltage levels) for periods of time. In some instances, a single voltage level is used for deprotection of polynucleotides. In some instances, more than one voltage level is used during deprotection. In some instances, a cathode voltage is kept constant at 0V, while the anode voltage is increased from 0V to 2V during a “pulse”. In some instances, a cathode voltage is kept constant at a negative voltage (e.g., −1V or other negative voltage), while the anode voltage is increased from 0V to 2V during a “pulse”. In some instances, a cathode voltage is decreased from 0V to a negative voltage (such as −1V), and an anode voltage is increased from 0V to 1V during a “pulse”. In some instances such voltages are synchronized, wherein the decrease in voltage at the cathode and increase in voltage at the anode occur at approximately the same time. In some instances such voltages are synchronized, wherein the decrease in voltage at the cathode and increase in voltage at the anode occur within 1 sec, 0.5 sec, 0.1 sec, 0.05 sec, 0.01 sec, 0.005 sec, 0.001 sec, or occur within 0.0005 sec of each other. In some instances, two voltage levels are used during a deprotection step. In some instances, two voltage levels are used during a deprotection step, e.g., a positive and neutral voltage. In some instances, two voltage levels are used during a deprotection step, e.g., a positive and negative voltage. In some instances, three voltage levels are used during a deprotection step, e.g., a positive, neutral (or zero/about zero), and negative voltage. Voltage in some instances is applied to multiple electrodes in fluid communication with the same surface, for example between a deblocking electrode and a shield electrode. Voltages between the deblocking electrode and shield electrode are in some instances are synchronized. In some instances, when the difference between the cathode and anode voltages exceeds a threshold, acid or other reagent is generated. In some instances, synchronizing positive anode and negative cathode voltages results in the advantage of reducing the magnitude of the voltages that are necessary to drive a device.

Devices may be described as circuits between an anode and a cathode. In some instances, such circuits are described as being in device states, such as “on”, “off”, or “alternate resistance”. In some instances, alternate resistance is a high resistance state, or “disconnect” state. In some instances, a high resistance state is a resistance state that is higher than an off state (e.g., low/no voltage in off state, but still connected to a ground). In some instances, a high resistance state provides an effective amount of resistance to reduce current flow through one or more inactive devices. Without being bound by theory, the disconnect state in some instances reduces undesired deprotection at areas adjacent to an on device. In some instances, a high resistance state provides an effective amount of resistance to reduce current flow to near zero in one or more inactive devices. In some instances an off state is generated by zero (or near zero) voltage between an inactive device and a common cathode. In some instances an off state exists even with a minimum voltage applied between an inactive device and a common cathode, wherein the minimum voltage is below that amount needed for deprotection. In some instances a high resistance state is generated by zero voltage between an inactive device and the cathode and a higher resistance between the inactive devices and a common cathode. In some instances, an off state indicates zero voltage or negative voltage between the anode and active device (cathode). In some instances, an on state indicates positive voltage between the anode and active device (cathode) which is sufficient for deprotection. In some instances, an inactive device is in the off or alternate resistance state. In some instances, an active device (where deprotection is desired) is cycled (pulsed) between one or more on and off states for a period of time. In some instances, an active device (where deprotection is desired) is cycled between one or more on and off states for a period of time and neighboring inactive devices are maintained in an alternative resistance state.

A voltage may be applied to the cathode in addition to the anode. In some instances, the cathode is biased with a negative voltage relative to ground. In some instances, biasing the voltage (bias voltage) of the cathode reduces the maximum anode voltage needed for electrochemical deprotection (e.g., the voltage difference between the anode and cathode will equal the anode voltage plus the magnitude of the negative bias voltage at the cathode). In some instances, a device comprises a contact bias on the cathode. In some instances, a bias voltage at the cathode is switched whenever the anode voltage is switched (e.g., synched). In some instances, a cathode controls electrochemistry for a single device. In some instances, a cathode controls electrochemistry for a plurality of devices (“common” cathode). In some instances, use of a common cathode results fewer transistors needed per device. In some instances, the bias voltage is no more than −0.1, −0.2, −0.3, −0.5, −0.7, −0.9, −1.0-1.1, −1.2, −1.5, −1.8, −2.0, −2.1, −2.2, or no more than −2.5 volts. In some instances, the biased voltage is at least −0.1, −0.2, −0.3, −0.5, −0.7, −0.9, −1.0-1.1, −1.2, −1.5, −1.8, −2.0, −2.1, −2.2, or at least −2.5 volts. In some instances, the biased voltage is about −0.1, −0.2, −0.3, −0.5, −0.7, −0.9, −1.0-1.1, −1.2, −1.5, −1.8, −2.0, −2.1, −2.2, or about −2.5 volts. In some instances, the biased voltage is −0.1 to −2.5 volts, −0.2 to −2.5 volts, −0.5 to −2.5 volts, −1.0 to −2.5 volts, −1.5 to −2.5 volts, −1.0 to −2.0 volts, −0.5 to −1.0 volts, −0.2 to −1.5 volts, or −2.0 to −2.5 volts.

The voltage between two layers of a device or surface may be varied. In some instances, a voltage is between the anode and cathode. In some instances the voltage is 0.5-3, 1-3, 1.5-2.5, 1-2.5, or 1.5-2 volts. In some instances, the voltage is at least 0.5, 0.75, 1, 1.2, 1.5, 1.7, 1.9, 2, 2.2, 2.4, or more than 2.4 volts. In some instances, the voltage is about 0.5, 0.75, 1, 1.2, 1.5, 1.7, 1.9, 2, 2.2, 2.4, or about 2.4 volts. In some instances, the voltage is −0.1 to −2.5 volts, −0.2 to −2.5 volts, −0.5 to −2.5 volts, −1.0 to −2.5 volts, −1.5 to −2.5 volts, −1.0 to −2.0 volts, −0.5 to −1.0 volts, −0.2 to −1.5 volts, or −2.0 to −2.5 volts. In some instances, a conducting layer of a device is charged with a positive voltage. In some instances, a conducting layer of a device is charged with a negative voltage. In some instances, a first layer of a device is charged with a positive voltage, and a second layer is charged with a negative voltage at the same time.

The (total) amount of time a voltage is applied may be varied for each synthesis cycle (e.g., deblocking, coupling, etc.). Voltage is applied in some instances for no more than 0.1, 0.2, 0.5, 0.8, 1, 2, 5, or no more than 10 seconds. Voltage is applied in some instances for 0.1-10, 0.5-10, 0.5-5, 0.1-5, 2-5, 2-10, 3-10, or 0.1-2 seconds. Voltage is applied in some instances about 0.1, 0.2, 0.5, 0.8, 1, 2, 5, or about 10 seconds. Voltage is applied in some instances for no more than 0.1, 0.2, 0.5, 0.8, 1, 2, 5, 10, 20, 50, 100, 200, 500, 800, or no more than 1000 milliseconds (ms). Voltage is applied in some instances for about 0.1, 0.2, 0.5, 0.8. 1, 2, 5, 10, 20, 50, 100, 200, 500, 800, or about 1000 milliseconds. Voltage is applied in some instances for 0.1-1000, 0.5-500, 0.5-50, 0.1-5, 2-50, 2-100, 3-200, 0.1-10, 1-100, 1-50, or 0.1-2 milliseconds.

Voltage may be applied as a single “on”/“off” cycle, or applied as a series of alternating “on” and “off” cycles to an active device. In some instances an “on” state is a positive voltage or a negative voltage. The application of voltage in the “on” state followed by an “off” state is in some instances defined as a “pulse.” In some instances, voltage is applied in a series of pulses, such as no more than 1, 2, 3, 4, 5, 6, 7, 8, 10, 20, 50, 80, 100, 110, 120, 150, 180, 200, 220, 250, 300, 500, or more than 500 pulses. In some instances, voltage is applied in a series of pulses, such as at least 1, 2, 3, 4, 5, 6, 7, 8, 10, 20, 50, 80, 100, 110, 120, 150, 180, 200, 220, 250, 300, 500, or at least 500 pulses. In some instances, voltage is applied in a series of pulses, such as 1-1000, 1-500, 1-300, 10-500, 10-100, 50-500, 50-200, 100-1000, 2-10, 2-8, 20-200, or 300-750 pulses. In some instances, voltage is applied in a series of pulses, such as about 1, 2, 3, 4, 5, 6, 7, 8, 10, 20, 50, 80, 100, 110, 120, 150, 180, 200, 220, 250, 300, 500, or about 500 pulses. In some instances, voltage is applied in a series of pulses, such as no more than 100, 200, 500, 800, 1000, 2000, 5000, 8000, 10000, 11000, 12000, 15000, 18000, 20000, 50000, 80000, 100,000, 200,000, 500,000, 800,000, or more than 1,000,000 pulses. In some instances, voltage is applied in a series of pulses, such as at least 100, 200, 500, 800, 1000, 2000, 5000, 8000, 10000, 11000, 12000, 15000, 18000, 20000, 50000, 80000, 100,000, 200,000, 500,000, 800,000, or at least 1,000,000 pulses. In some instances, voltage is applied in a series of pulses, such as about 100, 200, 500, 800, 1000, 2000, 5000, 8000, 10000, 11000, 12000, 15000, 18000, 20000, 50000, 80000, 100,000, 200,000, 500,000, 800,000, or about 1,000,000 pulses. In some instances, voltage is applied in a series of pulses, such as at least 10-1000, 10-5000, 100-10,000, 1000-50,000, 10000-100,000, 50000-500,000, 50000-1,000,000, 10,000-100,000 or 500,000-1,000,000 pulses.

The voltage application time may be divided by the number of pulses to define a pulse time (or pulse width, or time per pulse). In some instances a pulse time is no more than 0.1, 0.2, 0.5, 0.8. 1, 2, 5, 10, 20, 50, 100, 200, 500, 800, or no more than 1000 milliseconds. In some instances, a pulse time is 0 seconds. In some instances, a pulse time is greater than 0 seconds. In some instances a pulse time is no more than 0.1, 0.2, 0.5, 0.8. 1, 2, 5, 10, 20, 50, 100, 200, 500, 800, 1000, 2000, or no more than 5000 seconds. In some instances a pulse time is about 0.1, 0.2, 0.5, 0.8. 1, 2, 5, 10, 20, 50, 100, 200, 500, 800, or about 1000 milliseconds. In some instances a pulse time is about 0.1, 0.2, 0.5, 0.8. 1, 2, 5, 10, 20, 50, 100, 200, 500, 800, 1000, 2000, or no more than 5000 seconds. The pulse time in some instances is 0.1-1000, 0.5-500, 0.5-50, 0.1-5, 2-50, 2-100, 3-200, 0.1-10, 1-100, 1-50, or 0.1-2 milliseconds. The pulse time in some instances is 0.1-5000, 0.5-5000, 0.5-2000, 0.1-1000, 2-500, 2-100, 3-200, 0.1-10, 1-1000, 1-500, or 0.1-200 seconds. In some instances a pulse time is no more than 0.1, 0.2, 0.5, 0.8. 1, 2, 5, 10, 20, 50, 100, 200, 500, 800, or no more than 1000 microseconds. In some instances a pulse time is about 0.1, 0.2, 0.5, 0.8. 1, 2, 5, 10, 20, 50, 100, 200, 500, 800, or about 1000 microseconds. The pulse time in some instances is 0.1-1000, 0.5-500, 0.5-50, 0.1-5, 2-50, 2-100, 3-200, 0.1-10, 1-100, 1-50, or 0.1-2 microseconds. In some instances, a polynucleotide synthesis surface is washed with a solvent in between pulses. In some instances, a polynucleotide synthesis surface is not washed with a solvent in between pulses. In some instances a series of pulses are used to deliver voltage to a surface, followed by a wash step, followed by another series of pulses. Pulses need not be the same voltage. In some instances, a first pulse is positive, and a second pulse is negative. In some instances, the time between a positive and negative voltage is substantially instantaneous. In some instances, a first pulse is about 2 volts and a second pulse is about −0.6 volts. In some instances, a first pulse is 0.5 to 3 volts and a second pulse is −0.1 to −1.0 volts.

The time period between pulses may be varied to allow, without being bound by theory, electrochemically generated reagents to dissipate. The time between pulses in some instances is no more than 0.1, 0.2, 0.5, 0.8, 1, 2, 5, or no more than 10 seconds. The time between pulses in some instances is 0.1-10, 0.5-10, 0.5-5, 0.1-5, 2-5, 2-10, 3-10, or 0.1-2 seconds. The time between pulses in some instances is about 0.1, 0.2, 0.5, 0.8, 1, 2, 5, or about 10 seconds. The time between pulses in some instances is no more than 0.1, 0.2, 0.5, 0.8. 1, 2, 5, 10, 20, 50, 100, 200, 500, 800, or no more than 1000 milliseconds (ms). The time between pulses in some instances is about 0.1, 0.2, 0.5, 0.8. 1, 2, 5, 10, 20, 50, 100, 200, 500, 800, or about 1000 milliseconds. The time between pulses in some instances is 0.1-1000, 0.5-500, 0.5-50, 0.1-5, 2-50, 2-100, 3-200, 0.1-10, 1-100, 1-50, or 0.1-2 milliseconds. The time between pulses in some instances is no more than 0.1, 0.2, 0.5, 0.8. 1, 2, 5, 10, 20, 50, 100, 200, 500, 800, or no more than 1000 microseconds (ms). The time between pulses in some instances is about 0.1, 0.2, 0.5, 0.8. 1, 2, 5, 10, 20, 50, 100, 200, 500, 800, or about 1000 microseconds. The time between pulses in some instances is 0.1-1000, 0.5-500, 0.5-50, 0.1-5, 2-50, 2-100, 3-200, 0.1-10, 1-100, 1-50, or 0.1-2 microseconds. In some instances, the ratio between on and off times for series of pulses is described as a duty cycle. In some instances, a duty cycle is about 1:100, 1:50, 1:20, 1:10, 1:5, 1:2, 1:1.5, 1:1.05, 1.05:1, 1.5:1, 2:1, or about 3:1. In some instances, a duty cycle is no more than 1:100, 1:50, 1:20, 1:10, 1:5, 1:2, 1:1.5, 1:1.05, 1.05:1, 1.5:1, 2:1, or no more than 3:1. In some instances, a duty cycle is at least 1:100, 1:50, 1:20, 1:10, 1:5, 1:2, 1:1.5, 1:1.05, 1.05:1, 1.5:1, 2:1, or at least 3:1.

Various chemical reactions may be used to deblock polynucleotides, directly or indirectly. In some instances electrical voltage oxidizes or reduces a blocking group (protecting group) directly on a polynucleotide, causing the polynucleotide to be deblocked. In some instances, the voltage generates an in-situ reagent which deblocks the blocked nucleotide. In some instances, the polynucleotide comprises an acid-cleavable blocking group. In some instances, the reagent is dissolved in a solvent. In some instances, the reagent is an acid, such as H+. Various reagents may be used to electrochemically generate deblocking reagents such as acids. In some instances, a reagent comprises a quinone. In some instances, the quinone is benzoquinone, hydroquinone, anthraquinone, substituted benzoquinone, a hydrazine, a diazirine, or other reagent configured to generate an acid when a voltage is applied. In some instances, the reagent comprises a mixture of hydroquinone (HQ) and benzoquinone (BQ). In some instances, the reagent comprises a mixture of hydroquinone and benzoquinone, wherein the ratio of HQ:BQ is about 100:1, 50:1, 20:1, 10:1, 8:1, 5:1, 3:1, 2:1, 1:1, 1:2, 1:5, or about 1:10. In some instances, the ratio of HQ:BQ is at least 100:1, 50:1, 20:1, 10:1, 8:1, 5:1, 3:1, 2:1, or at least 1:1. In some instances, the ratio of HQ:BQ is no more than 100:1, 50:1, 20:1, 10:1, 8:1, 5:1, 3:1, 2:1, or no more than 1:1. In some instances, the ratio of HQ:BQ is 100:1-10:1, 50:1-1:1, 20:1-5:1, 15:1-5:1, 10:1-1:1, 10:1-2:1. 20:1-2:1, 1:1-1:5, 1:1-10:1 10:1-1:10, or 5:1-1:5.

Provided herein are methods of fabricating the devices and surfaces for polynucleotide synthesis. Described herein are layers integrated into a solid support. In some instances, layers comprise electrodes or are configured for use as electrodes. In some instances, electrodes are configured as cathodes or anodes. In some instances, an anode comprises a metal oxide. In some instances, nucleic acids are synthesized on an anode. In some instances, nucleic acids are synthesized on a metal oxide layer. In some instances, nucleic acids are synthesized on a porous metal oxide layer comprising a continuous metal layer beneath it. Electrodes in some instances comprise at least one conductor, and are fabricated of materials well known in the art. In some instances, electrodes comprise at least one conductor and one or more insulators or semi-conductors. Materials may comprise metals, non-metals, mixed-metal oxides, nitrides, carbides, silicon-based materials, or other material. In some instances, metal oxides include TiO₂, Ta₂O₅, IrO₂, RuO₂, RhO₂, Nb₂O₅, Al₂O₃, BaO, Y₂O₃, HfO₂, SrO or other metal oxide known in the art. In some instances, metal carbides include TiC, WC, ThC₂, ThC, VC, W₂C, ZrC, HfC, NbC, TaC, Ta₂C, or other metal carbide known in the art. In some instances, metal nitrides include GaN, InN, BN, Be₃N₂, Cr₂N, MoN, Si₃N₄, TaN, Th₂N₂, VN, ZrN, TiN, HfN, NbC, WN, TaN, or other metal nitride known in the art. In some instances, a device disclosed herein is manufactured with a combination of materials listed herein or any other suitable material known in the art.

Solid supports comprising layers may be coated with additional materials such as semiconductors or insulators. In some instances, a layer is configured for use as an electrode. In some instances, electrodes are coated with materials for polynucleotide attachment and synthesis. Each electrode can control one, or a plurality of different loci for synthesis, wherein each locus for synthesis has a density of polynucleotides. In some instances, the density is at least 1 oligo per 10 nm², 20, 50, 100, 200, 500, 1,000, 2,000, 5,000 or at least 1 oligo per 10,000 nm². In some instances, the density is about 1 oligo per 10 nm² to about 1 oligo per 5,000 nm², about 1 oligo per 50 nm² to about 1 oligo per 500 nm², or about 1 oligo per 25 nm² to about 1 oligo per 75 nm². In some instances, the density of polynucleotides is about 1 oligo per 25 nm² to about 1 oligo per 75 nm².

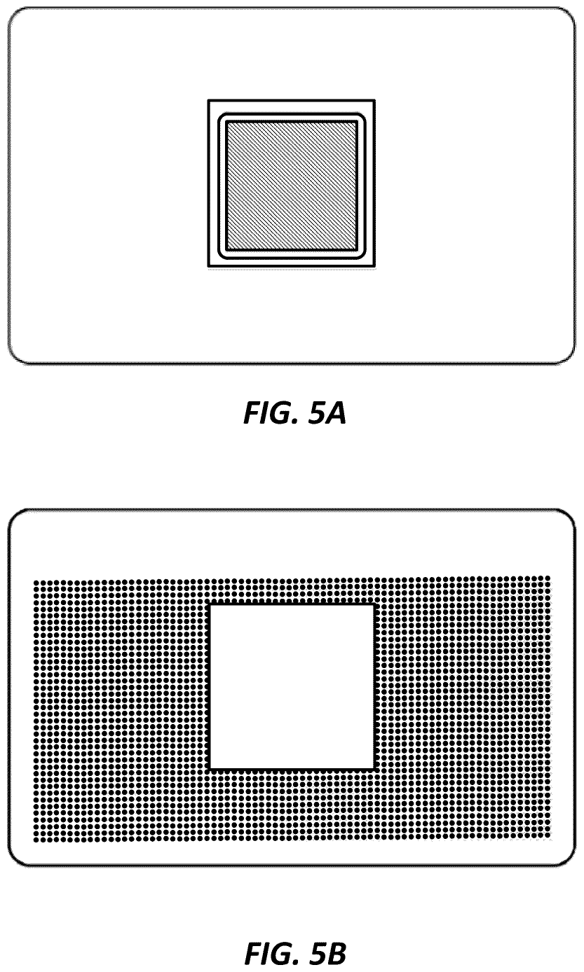

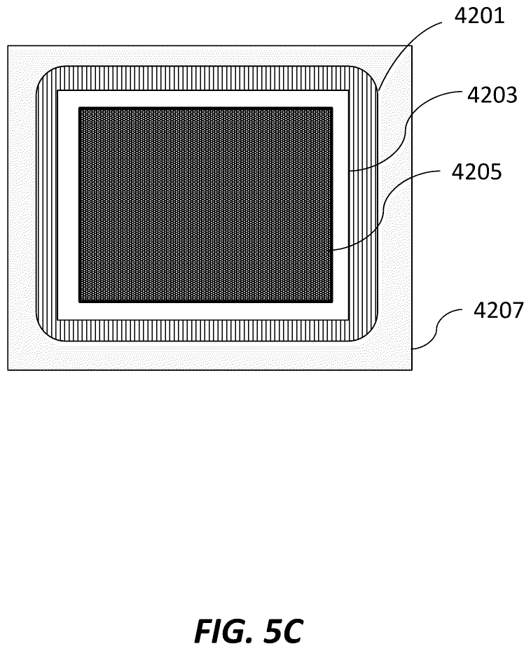

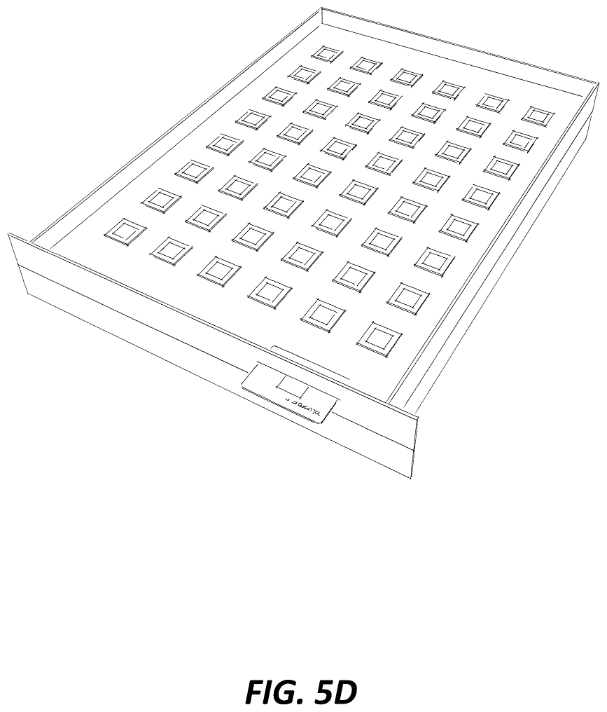

Described herein are devices wherein two or more solid supports are assembled. In some instances, solid supports are interfaced together on a larger unit. Interfacing may comprise exchange of fluids, electrical signals, or other medium of exchange between solid supports. This unit is capable of interface with any number of servers, computers, or networked devices. For example, a plurality of solid support is integrated onto a rack unit, which is conveniently inserted or removed from a server rack. The rack unit may comprise any number of solid supports. In some instances the rack unit comprises at least 1, 2, 5, 10, 20, 50, 100, 200, 500, 1000, 2000, 5000, 10,000, 20,000, 50,000, 100,000 or more than 100,000 solid supports. In some instances, two or more solid supports are not interfaced with each other. Nucleic acids (and the information stored in them) present on solid supports can be accessed from the rack unit. See, e.g., FIG. 5D. In some instances, solid supports are present on solid supports such as chips (FIGS. 5A-5C). Access includes removal of polynucleotides from solid supports, direct analysis of polynucleotides on the solid support, or any other method which allows the information stored in the nucleic acids to be manipulated or identified. Information in some instances is accessed from a plurality of racks, a single rack, a single solid support in a rack, a portion of the solid support, or a single locus on a solid support. In various instances, access comprises interfacing nucleic acids with additional devices such as mass spectrometers, HPLC, sequencing instruments, PCR thermocyclers, or other device for manipulating nucleic acids. Access to nucleic acid information in some instances is achieved by cleavage of polynucleotides from all or a portion of a solid support. Cleavage in some instances comprises exposure to chemical reagents (ammonia or other reagent), electrical potential, radiation, heat, light, acoustics, or other form of energy capable of manipulating chemical bonds. In some instances, cleavage occurs by charging one or more electrodes in the vicinity of the polynucleotides. In some instances, electromagnetic radiation in the form of UV light is used for cleavage of polynucleotides. In some instances, a lamp is used for cleavage of polynucleotides, and a mask mediates exposure locations of the UV light to the surface. In some instances, a laser is used for cleavage of polynucleotides, and a shutter opened/closed state controls exposure of the UV light to the surface. In some instances, access to nucleic acid information (including removal/addition of racks, solid supports, reagents, nucleic acids, or other component) is completely automated.

Solid supports as described herein comprise an active area. In some instances, the active area comprises addressable solid supports, regions, or loci for nucleic acid synthesis. In some instances, the active area comprises addressable regions or loci for nucleic acid storage. In some instances, an active area is in fluid communication with solvents or other reagents. The active area comprises varying dimensions. For example, the dimension of the active area is between about 1 mm to about 50 mm by about 1 mm to about 50 mm. In some instances, the active area comprises a width of at least or about 0.5, 1, 1.5, 2, 2.5, 3, 5, 5, 10, 12, 14, 16, 18, 20, 25, 30, 35, 40, 45, 50, 60, 70, 80, or more than 80 mm. In some instances, the active area comprises a height of at least or about 0.5, 1, 1.5, 2, 2.5, 3, 5, 5, 10, 12, 14, 16, 18, 20, 25, 30, 35, 40, 45, 50, 60, 70, 80, or more than 80 mm. An exemplary active area within a solid support is seen in FIG. 5C. A package 4207 comprises an active area 4205 within a solid support 4203. The package 4207 also comprises a fluidics interface 4201.

Described herein are devices, compositions, systems and methods for solid support based nucleic acid synthesis and storage, wherein the solid support has a number of sites (e.g., spots) or positions for synthesis or storage. In some instances, the solid support comprises up to or about 10,000 by 10,000 positions in an area. In some instances, the solid support comprises between about 1000 and 20,000 by between about 1000 and 20,000 positions in an area. In some instances, the solid support comprises at least or about 10, 30, 50, 75, 100, 200, 300, 400, 500, 1000, 2000, 3000, 4000, 5000, 6000, 7000, 8000, 9000, 10,000, 12,000, 14,000, 16,000, 18,000, 20,000 positions by least or about 10, 30, 50, 75, 100, 200, 300, 400, 500, 1000, 2000, 3000, 4000, 5000, 6000, 7000, 8000, 9000, 10,000, 12,000, 14,000, 16,000, 18,000, 20,000 positions in an area. In some instances the area is up to 0.25, 0.5, 0.75, 1.0, 1.25, 1.5, or 2.0 inches squared. In some instances, the solid support comprises addressable loci having a pitch of at least or about 0.1, 0.2, 0.25, 0.3, 0.4, 0.5, 1.0, 1.5, 2.0, 2.5, 3.0, 3.5, 4.0, 4.5, 5, 6, 7, 8, 9, 10, or more than 10 μm. In some instances, the solid support comprises addressable loci having a pitch of about 5 μm. In some instances, the solid support comprises addressable loci having a pitch of about 2 μm. In some instances, the solid support comprises addressable loci having a pitch of about 1 μm. In some instances, the solid support comprises addressable loci having a pitch of about 0.2 um. In some instances, the solid support comprises addressable loci having a pitch of about 0.2 um to about 10 μm, about 0.2 to about 8 μm, about 0.5 to about 10 μm, about 1 μm to about 10 μm, about 2 μm to about 8 μm, about 3 μm to about 5 μm, about 1 μm to about 3 μm or about 0.5 um to about 3 μm. In some instances, the solid support comprises addressable loci having a pitch of about 0.1 um to about 3 μm. In some instances, the solid support comprises addressable loci having a pitch of at least or about 0.01, 0.02, 0.025, 0.03, 0.04, 0.05, 0.1, 0.15, 0.02, 0.25, 0.30, 0.35, 0.4, 0.45, 0.5, 0.6, 0.7, 0.8, 0.9, 1, or more than 1 μm. In some instances, the solid support comprises addressable loci having a pitch of about 0.5 um. In some instances, the solid support comprises addressable loci having a pitch of about 0.2 um. In some instances, the solid support comprises addressable loci having a pitch of about 0.1 um. In some instances, the solid support comprises addressable loci having a pitch of about 0.02 um. In some instances, the solid support comprises addressable loci having a pitch of about 0.02 um to about 1 μm, about 0.02 to about 0.8 um, about 0.05 to about 0.1 um, about 0.1 um to about 1 μm, about 0.2 um to about 0.8 um, about 0.3 um to about 0.5 um, about 0.1 um to about 0.3 um or about 0.05 um to about 0.3 um. In some instances, the solid support comprises addressable loci having a pitch of about 0.01 um to about 0.3 um.

The solid support for nucleic acid synthesis or storage as described herein comprises a high capacity for storage of data. For example, the capacity of the solid support is at least or about 1, 2, 3, 4, 5, 6, 7, 8, 9, 10, 20, 50, 100, 200, 300, 400, 500, 600, 700, 800, 900, 1000, or more than 1000 petabytes. In some instances, the capacity of the solid support is between about 1 to about 10 petabytes or between about 1 to about 100 petabytes. In some instances, the capacity of the solid support is about 100 petabytes. In some instances, the data is stored as addressable arrays of packets as droplets. In some instances, the data is stored as addressable arrays of packets as droplets on a spot. In some instances, the data is stored as addressable arrays of packets as dry wells. In some instances, the addressable arrays comprise at least or about 1, 2, 3, 4, 5, 6, 7, 8, 9, 10, 20, 50, 100, 200, or more than 200 gigabytes of data. In some instances, the addressable arrays comprise at least or about 1, 2, 3, 4, 5, 6, 7, 8, 9, 10, 20, 50, 100, 200, or more than 200 terabytes of data. In some instances, an item of information is stored in a background of data. For example, an item of information encodes for about 10 to about 100 megabytes of data and is stored in 1 petabyte of background data. In some instances, an item of information encodes for at least or about 1, 10, 20, 30, 40, 50, 60, 70, 80, 90, 100, 150, 200, 300, 400, 500, or more than 500 megabytes of data and is stored in 1, 10, 20, 30, 40, 50, 60, 70, 80, 90, 100, 150, 200, 300, 400, 500, or more than 500 petabytes of background data.

### Nucleic Acid Based Information Storage

Provided herein are devices, compositions, systems and methods for nucleic acid-based information (data) storage. In a first step, a digital sequence encoding an item of information (i.e., digital information in a binary code for processing by a computer) is received. An encryption scheme is applied to convert the digital sequence from a binary code to a nucleic acid sequence. A surface material for nucleic acid extension, a design for loci for nucleic acid extension (aka, arrangement spots), and reagents for nucleic acid synthesis are selected. The surface of a structure is prepared for nucleic acid synthesis. De novo polynucleotide synthesis is performed. The synthesized polynucleotides are stored and available for subsequent release, in whole or in part. Once released, the polynucleotides, in whole or in part, are sequenced, subject to decryption to convert nucleic sequence back to digital sequence. The digital sequence is then assembled to obtain an alignment encoding for the original item of information.

### Items of Information

Optionally, an early step of data storage process disclosed herein includes obtaining or receiving one or more items of information in the form of an initial code. Items of information include, without limitation, text, audio and visual information. Exemplary sources for items of information include, without limitation, books, periodicals, electronic databases, medical records, letters, forms, voice recordings, animal recordings, biological profiles, broadcasts, films, short videos, emails, bookkeeping phone logs, internet activity logs, drawings, paintings, prints, photographs, pixelated graphics, and software code. Exemplary biological profile sources for items of information include, without limitation, gene libraries, genomes, gene expression data, and protein activity data. Exemplary formats for items of information include, without limitation, .txt, .PDF, .doc, .docx, .ppt, .pptx, .xls, .xlsx, .rtf, .jpg, gif, .psd, .bmp, tiff, .png, and .mpeg. The amount of individual file sizes encoding for an item of information, or a plurality of files encoding for items of information, in digital format include, without limitation, up to 1024 bytes (equal to 1 KB), 1024 KB (equal to 1 MB), 1024 MB (equal to 1 GB), 1024 GB (equal to 1 TB), 1024 TB (equal to 1 PB), 1 exabyte, 1 zettabyte, 1 yottabyte, 1 xenottabyte or more. In some instances, an amount of digital information is at least 1 gigabyte (GB). In some instances, the amount of digital information is at least 1, 2, 3, 4, 5, 6, 7, 8, 9, 10, 20, 50, 100, 200, 300, 400, 500, 600, 700, 800, 900, 1000 or more than 1000 gigabytes. In some instances, the amount of digital information is at least 1 terabyte (TB). In some instances, the amount of digital information is at least 1, 2, 3, 4, 5, 6, 7, 8, 9, 10, 20, 50, 100, 200, 300, 400, 500, 600, 700, 800, 900, 1000 or more than 1000 terabytes. In some instances, the amount of digital information is at least 1 petabyte (PB). In some instances, the amount of digital information is at least 1, 2, 3, 4, 5, 6, 7, 8, 9, 10, 20, 50, 100, 200, 300, 400, 500, 600, 700, 800, 900, 1000 or more than 1000 petabytes. In some instances, items of information are encoded using with an encoding scheme (e.g., 2 bits per base, 3 bits per base, or other encoding scheme).

### Structures for Polynucleotide Synthesis

Provided herein are rigid or flexibles structures for polynucleotide synthesis. In the case of rigid structures, provided herein are devices having a structure for the generation of a library of polynucleotides. In some instances, the structure comprises a plate.

In the case of flexible structures, provided herein are devices wherein the flexible structure comprises a continuous loop wrapped around one or more fixed structures, e.g., a pair of rollers or a non-continuous flexible structure wrapped around separate fixed structures, e.g., a pair reels. In some instances, the structures comprise multiple regions for polynucleotide synthesis. An exemplary structure comprises a plate with distinct regions for polynucleotide synthesis. The distinct regions may be separated by breaking or cutting. Each of the distinct regions may be further released, sequenced, decrypted, and read or stored. An alternative structure comprises a tape comprises with distinct regions for polynucleotide synthesis. The distinct regions may be separated by breaking or cutting. Each of the distinct regions may be further released, sequenced, decrypted, and read or stored. Provided herein are flexible structures having a surface with a plurality of loci for polynucleotide extension. Each locus in a portion of the flexible structure, may be a substantially planar spot (e.g., flat), a channel, or a well. In some instances, each locus of the structure has a width of about 10 μm and a distance between the center of each structure of about 21 μm. In some instances, each locus of the structure has a width of about 1 μm and a distance between the center of each structure of about 2 μm. In some instances, each locus of the structure has a width of about 0.1 um and a distance between the center of each structure of about 0.2 um. Loci may comprise, without limitation, circular, rectangular, tapered, or rounded shapes. Alternatively or in combination, the structures are rigid. In some instances, the rigid structures comprise loci for polynucleotide synthesis. In some instances, the rigid structures comprise substantially planar regions, channels, or wells for polynucleotide synthesis.

In some instances, a well described herein has a width to depth (or height) ratio of 1 to 0.01, wherein the width is a measurement of the width at the narrowest segment of the well. In some instances, a well described herein has a width to depth (or height) ratio of 0.5 to 0.01, wherein the width is a measurement of the width at the narrowest segment of the well. In some instances, a well described herein has a width to depth (or height) ratio of about 0.01, 0.05, 0.1, 0.15, 0.16, 0.2, 0.5, or 1. Provided herein are structures for polynucleotide synthesis comprising a plurality of discrete loci for polynucleotide synthesis. Exemplary structures for the loci include, without limitation, substantially planar regions, channels, wells or protrusions. Structures described herein are may comprise a plurality of clusters, each cluster comprising a plurality of wells, loci or channels. Alternatively, described herein are may comprise a homogenous arrangement of wells, loci or channels. Structures provided herein may comprise wells having a height or depth from about 5 μm to about 500 μm, from about 5 μm to about 400 μm, from about 5 μm to about 300 μm, from about 5 μm to about 200 μm, from about 5 μm to about 100 μm, from about 5 μm to about 50 μm, or from about 10 μm to about 50 μm. In some instances, the height of a well is less than 100 μm, less than 80 μm, less than 60 μm, less than 40 μm or less than 20 μm. In some instances, well height is about 10, 20, 30, 40, 50, 60, 70, 80, 90, 100, 200, 300, 400, 500 μm or more. In some instances, the height or depth of the well is at least 10, 25, 50, 75, 100, 200, 300, 400, 500, 600, 700, 800, 900, 1000, or more than 1000 nm. In some instances, the height or depth of the well is in a range of about 10 nm to about 1000 nm, about 25 nm to about 900 nm, about 50 nm to about 800 nm, about 75 nm to about 700 nm, about 100 nm to about 600 nm, or about 200 nm to about 500. In some instances, the height or depth of the well is in a range of about 50 nm to about 1 μm. In some instances, well height is about 10, 20, 30, 40, 50, 60, 70, 80, 90, 100, 200, 300, 400, 500, 700, 800, 900 or about 1000 nm.

Structures for polynucleotide synthesis provided herein may comprise channels. The channels may have a width to depth (or height) ratio of 1 to 0.01, wherein the width is a measurement of the width at the narrowest segment of the microchannel. In some instances, a channel described herein has a width to depth (or height) ratio of 0.5 to 0.01, wherein the width is a measurement of the width at the narrowest segment of the microchannel. In some instances, a channel described herein has a width to depth (or height) ratio of about 0.01, 0.05, 0.1, 0.15, 0.16, 0.2, 0.5, or 1.

Described herein are structures for polynucleotide synthesis comprising a plurality of discrete loci. Structures comprise, without limitation, substantially planar regions, channels, protrusions, or wells for polynucleotide synthesis. In some instances, structures described herein are provided comprising a plurality of channels, wherein the height or depth of the channel is from about 5 μm to about 500 μm, from about 5 μm to about 400 μm, from about 5 μm to about 300 μm, from about 5 μm to about 200 μm, from about 5 μm to about 100 μm, from about 5 μm to about 50 μm, or from about 10 μm to about 50 μm. In some cases, the height of a channel is less than 100 μm, less than 80 μm, less than 60 μm, less than 40 μm or less than 20 μm. In some cases, channel height is about 10, 20, 30, 40, 50, 60, 70, 80, 90, 100, 200, 300, 400, 500 μm or more. In some instances, the height or depth of the channel is at least 10, 25, 50, 75, 100, 200, 300, 400, 500, 600, 700, 800, 900, 1000, or more than 1000 nm. In some instances, the height or depth of the channel is in a range of about 10 nm to about 1000 nm, about 25 nm to about 900 nm, about 50 nm to about 800 nm, about 75 nm to about 700 nm, about 100 nm to about 600 nm, or about 200 nm to about 500. Channels described herein may be arranged on a surface in clusters or as a homogenous field.

The width of a locus on the surface of a structure for polynucleotide synthesis described herein may be from about 0.1 um to about 500 μm, from about 0.5 um to about 500 μm, from about 1 μm to about 200 μm, from about 1 μm to about 100 μm, from about 5 μm to about 100 μm, or from about 0.1 um to about 100 μm, for example, about 90 μm, 80 um, 70 μm, 60 um, 50 μm, 40 um, 30 μm, 20 um, 10 μm, 5 um, 1 μm or 0.5 um. In some instances, the width of a locus is less than about 100 μm, 90 um, 80 μm, 70 um, 60 μm, 50 um, 40 μm, 30 um, 20 μm or 10 μm. In some instances, the width of a locus is at least 10, 25, 50, 75, 100, 200, 300, 400, 500, 600, 700, 800, 900, 1000, or more than 1000 nm. In some instances, the width of a locus is in a range of about 10 nm to about 1000 nm, about 25 nm to about 900 nm, about 50 nm to about 800 nm, about 75 nm to about 700 nm, about 100 nm to about 600 nm, or about 200 nm to about 500. In some instances, the width of a locus is in a range of about 50 nm to about 1000 nm. In some instances, the distance between the center of two adjacent loci is from about 0.1 um to about 500 um, 0.5 um to about 500 um, from about 1 um to about 200 um, from about 1 um to about 100 um, from about 5 um to about 200 um, from about 5 um to about 100 um, from about 5 um to about 50 um, or from about 5 um to about 30 um, for example, about 20 um. In some instances, the total width of a locus is about 5 um, 10 um, 20 um, 30 um, 40 um, 50 um, 60 um, 70 um, 80 um, 90 um, or 100 um. In some instances, the total width of a locus is about 1 um to 100 um, 30 um to 100 um, or 50 um to 70 um. In some instances, the distance between the center of two adjacent loci is from about 0.5 um to about 2 um, 0.5 um to about 2 um, from about 0.75 um to about 2 um, from about 1 um to about 2 um, from about 0.2 um to about 1 um, from about 0.5 um to about 1.5 um, from about 0.5 um to about 0.8 um, or from about 0.5 um to about 1 um, for example, about 1 um. In some instances, the total width of a locus is about 50 nm, 0.1 um, 0.2 um, 0.3 um, 0.4 um, 0.5 um, 0.6 um, 0.7 um, 0.8 um, 0.9 um, 1 μm, 1.1 um, 1.2 um, 1.3 um, 1.4 um, or 1.5 um. In some instances, the total width of a locus is about 0.5 um to 2 μm, 0.75 um to 1 μm, or 0.9 um to 2 um.

In some instances, each locus supports the synthesis of a population of polynucleotides having a different sequence than a population of polynucleotides grown on another locus. Provided herein are surfaces which comprise at least 10, 100, 256, 500, 1000, 2000, 3000, 4000, 5000, 6000, 7000, 8000, 9000, 10000, 11000, 12000, 13000, 14000, 15000, 20000, 30000, 40000, 50000 or more clusters. Provided herein are surfaces which comprise more than 2,000; 5,000; 10,000; 20,000; 30,000; 50,000; 100,000; 200,000; 300,000; 400,000; 500,000; 600,000; 700,000; 800,000; 900,000; 1,000,000; 5,000,000; or 10,000,000 or more distinct loci. In some cases, each cluster includes 1, 2, 3, 4, 5, 6, 7, 8, 9, 10, 20, 30, 40, 50, 60, 70, 80, 90, 100, 120, 130, 150, 200, 500 or more loci. In some cases, each cluster includes 50 to 500, 50 to 200, 50 to 150, or 100 to 150 loci. In some cases, each cluster includes 100 to 150 loci. In some instances, each cluster includes 109, 121, 130 or 137 loci.

Provided herein are loci having a width at the longest segment of 5 to 100 μm. In some cases, the loci have a width at the longest segment of about 30, 35, 40, 45, 50, 55 or 60 μm. In some cases, the loci are channels having multiple segments, wherein each segment has a center to center distance apart of 5 to 50 μm. In some cases, the center to center distance apart for each segment is about 5, 10, 15, 20 or 25 um.

Provided herein are loci having a width at the longest segment of 5 to 500 nm. In some cases, the loci have a width at the longest segment of about 30, 35, 40, 45, 50, 55, 60, 80, or 100 nm. In some cases, the loci are channels having multiple segments, wherein each segment has a center to center distance apart of 5 to 50 nm. In some cases, the center to center distance apart for each segment is about 5, 10, 15, 20, 25, 50, 100, or 200 nm.

In some instances, the number of distinct polynucleotides synthesized on the surface of a structure described herein is dependent on the number of distinct loci available in the substrate. In some instances, the density of loci within a cluster of a substrate is at least or about 1 locus per mm², 10 loci per mm², 25 loci per mm², 50 loci per mm², 65 loci per mm², 75 loci per mm², 100 loci per mm², 130 loci per mm², 150 loci per mm², 175 loci per mm², 200 loci per mm², 300 loci per mm², 400 loci per mm², 500 loci per mm², 1,000 loci per mm², 104 loci per mm², 105 loci per mm², 106 loci per mm², or more. In some cases, a substrate comprises from about 10 loci per mm² to about 500 mm², from about 25 loci per mm² to about 400 mm², from about 50 loci per mm² to about 500 mm², from about 100 loci per mm² to about 500 mm², from about 150 loci per mm² to about 500 mm², from about 10 loci per mm² to about 250 mm², from about 50 loci per mm² to about 250 mm², from about 10 loci per mm² to about 200 mm², or from about 50 loci per mm² to about 200 mm². In some cases, a substrate comprises from about 104 loci per mm² to about 105 mm². In some cases, a substrate comprises from about 105 loci per mm² to about 10⁷ mm². In some cases, a substrate comprises at least 105 loci per mm². In some cases, a substrate comprises at least 106 loci per mm². In some cases, a substrate comprises at least 10⁷ loci per mm². In some cases, a substrate comprises from about 104 loci per mm² to about 105 mm². In some instances, the density of loci within a cluster of a substrate is at least or about 1 locus per um², 10 loci per um², 25 loci per um², 50 loci per um², 65 loci per um², 75 loci per um², 100 loci per um², 130 loci per um², 150 loci per um², 175 loci per um², 200 loci per um², 300 loci per um², 400 loci per um², 500 loci per um², 1,000 loci per um² or more. In some cases, a substrate comprises from about 10 loci per um² to about 500 um², from about 25 loci per um² to about 400 um², from about 50 loci per um² to about 500 um², from about 100 loci per um² to about 500 um², from about 150 loci per um² to about 500 um², from about 10 loci per um² to about 250 um², from about 50 loci per um² to about 250 um², from about 10 loci per um² to about 200 um², or from about 50 loci per um² to about 200 um².

In some instances, the distance between the centers of two adjacent loci within a cluster is from about 10 um to about 500 um, from about 10 um to about 200 um, or from about 10 um to about 100 um. In some cases, the distance between two centers of adjacent loci is greater than about 10 um, 20 um, 30 um, 40 um, 50 um, 60 um, 70 um, 80 um, 90 um or 100 um. In some cases, the distance between the centers of two adjacent loci is less than about 200 um, 150 um, 100 um, 80 um, 70 um, 60 um, 50 um, 40 um, 30 um, 20 um or 10 um. In some cases, the distance between the centers of two adjacent loci is less than about 10000 nm, 8000 nm, 6000 nm, 4000 nm, 2000 nm 1000 nm, 800 nm, 600 nm, 400 nm, 200 nm, 150 nm, 100 nm, 80 um, 70 nm, 60 nm, 50 nm, 40 nm, 30 nm, 20 nm or 10 nm. In some instances, each square meter of a structure described herein allows for at least 10⁷, 10⁸, 10⁹, 10¹⁰, 10¹¹ loci, where each locus supports one polynucleotide. In some instances, 10⁹ polynucleotides are supported on less than about 6, 5, 4, 3, 2 or 1 m² of a structure described herein.

In some instances, a structure described herein provides support for the synthesis of more than 2,000; 5,000; 10,000; 20,000; 30,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more non-identical polynucleotides. In some cases, the structure provides support for the synthesis of more than 2,000; 5,000; 10,000; 20,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more polynucleotides encoding for distinct sequences. In some instances, at least a portion of the polynucleotides have an identical sequence or are configured to be synthesized with an identical sequence. In some instances, the structure provides a surface environment for the growth of polynucleotides having at least 50, 60, 70, 75, 80, 85, 90, 95, 100, 110, 120, 130, 140, 150, 160, 175, 200, 225, 250, 275, 300, 325, 350, 375, 400, 425, 450, 475, 500 bases or more. In some arrangements, structures for polynucleotide synthesis described herein comprise sites for polynucleotide synthesis in a uniform arrangement.

In some instances, polynucleotides are synthesized on distinct loci of a structure, wherein each locus supports the synthesis of a population of polynucleotides. In some cases, each locus supports the synthesis of a population of polynucleotides having a different sequence than a population of polynucleotides grown on another locus. In some instances, the loci of a structure are located within a plurality of clusters. In some instances, a structure comprises at least 10, 500, 1000, 2000, 3000, 4000, 5000, 6000, 7000, 8000, 9000, 10000, 11000, 12000, 13000, 14000, 15000, 20000, 30000, 40000, 50000 or more clusters. In some instances, a structure comprises more than 2,000; 5,000; 10,000; 100,000; 200,000; 300,000; 400,000; 500,000; 600,000; 700,000; 800,000; 900,000; 1,000,000; 1,100,000; 1,200,000; 1,300,000; 1,400,000; 1,500,000; 1,600,000; 1,700,000; 1,800,000; 1,900,000; 2,000,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; or 10,000,000 or more distinct loci. In some cases, each cluster includes 1, 2, 3, 4, 5, 6, 7, 8, 9, 10, 20, 30, 40, 50, 60, 70, 80, 90, 100, 120, 130, 150 or more loci. In some instances, each cluster includes 50 to 500, 100 to 150, or 100 to 200 loci. In some instances, each cluster includes 109, 121, 130 or 137 loci. In some instances, each cluster includes 5, 6, 7, 8, 9, 10, 11 or 12 loci. In some instances, polynucleotides from distinct loci within one cluster have sequences that, when assembled, encode for a contiguous longer polynucleotide of a predetermined sequence.

### Structure Size

In some instances, a structure described herein is about the size of a plate (e.g., chip), for example between about 40 and 120 mm by between about 25 and 100 mm. In some instances, a structure described herein has a diameter less than or equal to about 1000 mm, 500 mm, 450 mm, 400 mm, 300 mm, 250 nm, 200 mm, 150 mm, 100 mm or 50 mm. In some instances, the diameter of a substrate is between about 25 mm and 1000 mm, between about 25 mm and about 800 mm, between about 25 mm and about 600 mm, between about 25 mm and about 500 mm, between about 25 mm and about 400 mm, between about 25 mm and about 300 mm, or between about 25 mm and about 200. Non-limiting examples of substrate size include about 300 mm, 200 mm, 150 mm, 130 mm, 100 mm, 84 mm, 76 mm, 54 mm, 51 mm and 25 mm. In some instances, a substrate has a planar surface area of at least 100 mm²; 200 mm²; 500 mm²; 1,000 mm²; 2,000 mm²; 4,500 mm²; 5,000 mm²; 10,000 mm²; 12,000 mm²; 15,000 mm²; 20,000 mm²; 30,000 mm²; 40,000 mm²; 50,000 mm² or more. In some instances, the thickness is between about 50 mm and about 2000 mm, between about 50 mm and about 1000 mm, between about 100 mm and about 1000 mm, between about 200 mm and about 1000 mm, or between about 250 mm and about 1000 mm. Non-limiting examples thickness include 275 mm, 375 mm, 525 mm, 625 mm, 675 mm, 725 mm, 775 mm and 925 mm. In some instances, the thickness is at least or about 0.5 mm, 1.0 mm, 1.5 mm, 2.0 mm, 2.5 mm, 3.0 mm, 3.5 mm, 4.0 mm, or more than 4.0 mm. In some cases, the thickness of varies with diameter and depends on the composition of the substrate. For example, a structure comprising materials other than silicon may have a different thickness than a silicon structure of the same diameter. Structure thickness may be determined by the mechanical strength of the material used and the structure must be thick enough to support its own weight without cracking during handling. In some instances, a structure is more than about 1, 2, 3, 4, 5, 10, 15, 30, 40, 50 feet in any one dimension.

### Materials

Provided herein are devices comprising a surface, wherein the surface is modified to support polynucleotide synthesis at predetermined locations and with a resulting low error rate, a low dropout rate, a high yield, and a high oligo representation. In some instances, surfaces of devices for polynucleotide synthesis provided herein are fabricated from a variety of materials capable of modification to support a de novo polynucleotide synthesis reaction. In some cases, the devices are sufficiently conductive, e.g., are able to form uniform electric fields across all or a portion of the devices. Devices described herein may comprise a flexible material. Exemplary flexible materials include, without limitation, modified nylon, unmodified nylon, nitrocellulose, and polypropylene. Devices described herein may comprise a rigid material. Exemplary rigid materials include, without limitation, glass, fuse silica, silicon, silicon dioxide, silicon nitride, plastics (for example, polytetrafluoroethylene, polypropylene, polystyrene, polycarbonate, and blends thereof, and metals (for example, gold, platinum). Devices disclosed herein may be fabricated from a material comprising silicon, polystyrene, agarose, dextran, cellulosic polymers, polyacrylamides, polydimethylsiloxane (PDMS), glass, or any combination thereof. In some cases, devices disclosed herein are manufactured with a combination of materials listed herein or any other suitable material known in the art.

Devices described herein may comprise material having a range of tensile strength. Exemplary materials having a range of tensile strengths include, but are not limited to, nylon (70 MPa), nitrocellulose (1.5 MPa), polypropylene (40 MPa), silicon (268 MPa), polystyrene (40 MPa), agarose (1-10 MPa), polyacrylamide (1-10 MPa), polydimethylsiloxane (PDMS) (3.9-10.8 MPa). Solid supports described herein can have a tensile strength from 1 to 300, 1 to 40, 1 to 10, 1 to 5, or 3 to 11 MPa. Solid supports described herein can have a tensile strength of about 1, 1.5, 2, 3, 4, 5, 6, 7, 8, 9, 10, 11, 20, 25, 40, 50, 60, 70, 80, 90, 100, 150, 200, 250, 270, or more MPa. In some instances, a device described herein comprises a solid support for polynucleotide synthesis that is in the form of a flexible material capable of being stored in a continuous loop or reel, such as a tape or flexible sheet.

Young's modulus measures the resistance of a material to elastic (recoverable) deformation under load. Exemplary materials having a range of Young's modulus stiffness include, but are not limited to, nylon (3 GPa), nitrocellulose (1.5 GPa), polypropylene (2 GPa), silicon (150 GPa), polystyrene (3 GPa), agarose (1-10 GPa), polyacrylamide (1-10 GPa), polydimethylsiloxane (PDMS) (1-10 GPa). Solid supports described herein can have a Young's moduli from 1 to 500, 1 to 40, 1 to 10, 1 to 5, or 3 to 11 GPa. Solid supports described herein can have a Young's moduli of about 1, 1.5, 2, 3, 4, 5, 6, 7, 8, 9, 10, 11, 20, 25, 40, 50, 60, 70, 80, 90, 100, 150, 200, 250, 400, 500 GPa, or more. As the relationship between flexibility and stiffness are inverse to each other, a flexible material has a low Young's modulus and changes its shape considerably under load. In some instances, a solid support described herein has a surface with a flexibility of at least nylon.

In some cases, devices disclosed herein comprise a silicon dioxide base and a surface layer of silicon oxide. Alternatively, the devices may have a base of silicon oxide. Surface of the devices provided here may be textured, resulting in an increase overall surface area for polynucleotide synthesis. Devices disclosed herein in some instances comprise at least 5%, 10%, 25%, 50%, 80%, 90%, 95%, or 99% silicon. Devices disclosed herein in some instances are fabricated from silicon on insulator (SOI) wafer.

The structure may be fabricated from a variety of materials, suitable for the methods and compositions of the invention described herein. In instances, the materials from which the substrates/solid supports of the comprising the invention are fabricated exhibit a low level of polynucleotide binding. In some situations, material that are transparent to visible and/or UV light can be employed. Materials that are sufficiently conductive, e.g. those that can form uniform electric fields across all or a portion of the substrates/solids support described herein, can be utilized. In some instances, such materials may be connected to an electric ground. In some cases, the substrate or solid support can be heat conductive or insulated. The materials can be chemical resistant and heat resistant to support chemical or biochemical reactions such as a series of polynucleotide synthesis reactions. For flexible materials, materials of interest include nylon, both modified and unmodified, nitrocellulose, polypropylene, and the like.

For rigid materials, specific materials of interest include glass; fuse silica; silicon, plastics (for example polytetrafluoroethylene, polypropylene, polystyrene, polycarbonate, and blends thereof, and the like); metals (for example, gold, platinum, and the like). The structure can be fabricated from a material selected from the group consisting of silicon, polystyrene, agarose, dextran, cellulosic polymers, polyacrylamides, polydimethylsiloxane (PDMS), and glass. The substrates/solid supports or the microstructures, reactors therein may be manufactured with a combination of materials listed herein or any other suitable material known in the art.

In some instances, a substrate disclosed herein comprises a computer readable material. Computer readable materials include, without limitation, magnetic media, reel-to-reel tape, cartridge tape, cassette tape, flexible disk, paper media, film, microfiche, continuous tape (e.g., a belt) and any media suitable for storing electronic instructions. In some cases, the substrate comprises magnetic reel-to-reel tape or a magnetic belt. In some instances, the substrate comprises a flexible printed circuit board.

Structures described herein may be transparent to visible and/or UV light. In some instances, structures described herein are sufficiently conductive to form uniform electric fields across all or a portion of a structure. In some instances, structures described herein are heat conductive or insulated. In some instances, the structures are chemical resistant and heat resistant to support a chemical reaction such as a polynucleotide synthesis reaction. In some instances, the substrate is magnetic. In some instances, the structures comprise a metal or a metal alloy.

Structures for polynucleotide synthesis may be over 1, 2, 5, 10, 30, 50 or more feet long in any dimension. In the case of a flexible structure, the flexible structure is optionally stored in a wound state, e.g., in a reel. In the case of a large rigid structure, e.g., greater than 1 foot in length, the rigid structure can be stored vertically or horizontally.

### Surface Preparation

Provided herein are methods to support the immobilization of a biomolecule on a substrate, where a surface of a structure described herein comprises a material and/or is coated with a material that facilitates a coupling reaction with the biomolecule for attachment. To prepare a structure for biomolecule immobilization, surface modifications may be employed that chemically and/or physically alter the substrate surface by an additive or subtractive process to change one or more chemical and/or physical properties of a substrate surface or a selected site or region of the surface. For example, surface modification involves (1) changing the wetting properties of a surface, (2) functionalizing a surface, i.e. providing, modifying or substituting surface functional groups, (3) defunctionalizing a surface, i.e. removing surface functional groups, (4) otherwise altering the chemical composition of a surface, e.g., through etching, (5) increasing or decreasing surface roughness, (6) providing a coating on a surface, e.g., a coating that exhibits wetting properties that are different from the wetting properties of the surface, and/or (7) depositing particulates on a surface. In some instances, the surface of a structure is selectively functionalized to produce two or more distinct areas on a structure, wherein at least one area has a different surface or chemical property that another area of the same structure. Such properties include, without limitation, surface energy, chemical termination, surface concentration of a chemical moiety, and the like.

In some instances, a surface of a structure disclosed herein is modified to comprise one or more actively functionalized surfaces configured to bind to both the surface of the substrate and a biomolecule, thereby supporting a coupling reaction to the surface. In some instances, the surface is also functionalized with a passive material that does not efficiently bind the biomolecule, thereby preventing biomolecule attachment at sites where the passive functionalization agent is bound. In some cases, the surface comprises an active layer only defining distinct loci for biomolecule support.

In some instances, the surface is contacted with a mixture of functionalization groups which are in any different ratio. In some instances, a mixture comprises at least 2, 3, 4, 5 or more different types of functionalization agents. In some cases, the ratio of the at least two types of surface functionalization agents in a mixture is about 1:1, 1:2, 1:5, 1:10, 2:10, 3:10, 4:10, 5:10, 6:10, 7:10, 8:10, 9:10, or any other ratio to achieve a desired surface representation of two groups. In some instances, desired surface tensions, wettabilities, water contact angles, and/or contact angles for other suitable solvents are achieved by providing a substrate surface with a suitable ratio of functionalization agents. In some cases, the agents in a mixture are chosen from suitable reactive and inert moieties, thus diluting the surface density of reactive groups to a desired level for downstream reactions. In some instances, the mixture of functionalization reagents comprises one or more reagents that bind to a biomolecule and one or more reagents that do not bind to a biomolecule. Therefore, modulation of the reagents allows for the control of the amount of biomolecule binding that occurs at a distinct area of functionalization.

In some instances, a method for substrate functionalization comprises deposition of a silane molecule onto a surface of a substrate. The silane molecule may be deposited on a high energy surface of the substrate. In some instances the high surface energy region includes a passive functionalization reagent. Methods described herein provide for a silane group to bind the surface, while the rest of the molecule provides a distance from the surface and a free hydroxyl group at the end to which a biomolecule attaches. In some instances, the silane is an organofunctional alkoxysilane molecule. Non-limiting examples of organofunctional alkoxysilane molecules include dimethylchloro-octodecyl-silane, methyldichloro-octodecyl-silane, trichloro-octodecyl-silane, and trimethyl-octodecyl-silane, triethyl-octodecyl-silane. In some instances, the silane is an amino silane. Examples of amino silanes include, without limitation, 11-acetoxyundecyltriethoxysilane, n-decyltriethoxysilane, (3-aminopropyl)trimethoxysilane, (3-aminopropyl)triethoxysilane, glycidyloxypropyl/trimethoxysilane and N-(3-triethoxysilylpropyl)-4-hydroxybutyramide. In some instances, the silane comprises 11-acetoxyundecyltriethoxysilane, n-decyltriethoxysilane, (3-aminopropyl)trimethoxysilane, (3-aminopropyl)triethoxysilane, glycidyloxypropyl/trimethoxysilane, N-(3-triethoxysilylpropyl)-4-hydroxybutyramide, or any combination thereof. In some instances, an active functionalization agent comprises 11-acetoxyundecyltriethoxysilane. In some instances, an active functionalization agent comprises n-decyltriethoxysilane. In some cases, an active functionalization agent comprises glycidyloxypropyltriethoxysilane (GOPS). In some instances, the silane is a fluorosilane. In some instances, the silane is a hydrocarbon silane. In some cases, the silane is 3-iodo-propyltrimethoxysilane. In some cases, the silane is octylchlorosilane.

In some instances, silanization is performed on a surface through self-assembly with organofunctional alkoxysilane molecules. The organofunctional alkoxysilanes are classified according to their organic functions. Non-limiting examples of siloxane functionalizing reagents include hydroxyalkyl siloxanes (silylate surface, functionalizing with diborane and oxidizing the alcohol by hydrogen peroxide), diol (dihydroxyalkyl) siloxanes (silylate surface, and hydrolyzing to diol), aminoalkyl siloxanes (amines require no intermediate functionalizing step), glycidoxysilanes (3-glycidoxypropyl-dimethyl-ethoxysilane, glycidoxy-trimethoxysilane), mercaptosilanes (3-mercaptopropyl-trimethoxysilane, 3-4 epoxycyclohexyl-ethyltrimethoxysilane or 3-mercaptopropyl-methyl-dimethoxysilane), bicyclohepthenyl-trichlorosilane, butyl-aldehydr-trimethoxysilane, or dimeric secondary aminoalkyl siloxanes. Exemplary hydroxyalkyl siloxanes include allyl trichlorochlorosilane turning into 3-hydroxypropyl, or 7-oct-1-enyl trichlorochlorosilane turning into 8-hydroxyoctyl. The diol (dihydroxyalkyl) siloxanes include glycidyl trimethoxysilane-derived (2,3-dihydroxypropyloxy)propyl (GOPS). The aminoalkyl siloxanes include 3-aminopropyl trimethoxysilane turning into 3-aminopropyl (3-aminopropyl-triethoxysilane, 3-aminopropyl-diethoxy-methylsilane, 3-aminopropyl-dimethyl-ethoxysilane, or 3-aminopropyl-trimethoxysilane). In some cases, the dimeric secondary aminoalkyl siloxanes is bis(3-trimethoxysilylpropyl) amine turning into bis(silyloxylpropyl)amine.

Active functionalization areas may comprise one or more different species of silanes, for example, 1, 2, 3, 4, 5, 6, 7, 8, 9, 10 or more silanes. In some cases, one of the one or more silanes is present in the functionalization composition in an amount greater than another silane. For example, a mixed silane solution having two silanes comprises a 99:1, 98:2, 97:3, 96:4, 95:5, 94:6, 93:7, 92:8, 91:9, 90:10, 89:11, 88:12, 87:13, 86:14, 85:15, 84:16, 83:17, 82:18, 81:19, 80:20, 75:25, 70:30, 65:35, 60:40, 55:45 ratio of one silane to another silane. In some instances, an active functionalization agent comprises 11-acetoxyundecyltriethoxysilane and n-decyltriethoxysilane. In some instances, an active functionalization agent comprises 11-acetoxyundecyltriethoxysilane and n-decyltriethoxysilane in a ratio from about 20:80 to about 1:99, or about 10:90 to about 2:98, or about 5:95.

In some instances, functionalization comprises deposition of a functionalization agent to a structure by any deposition technique, including, but not limiting to, chemical vapor deposition (CVD), atomic layer deposition (ALD), plasma enhanced CVD (PECVD), plasma enhanced ALD (PEALD), metal organic CVD (MOCVD), hot wire CVD (HWCVD), initiated CVD (iCVD), modified CVD (MCVD), vapor axial deposition (VAD), outside vapor deposition (OVD), physical vapor deposition (e.g., sputter deposition, evaporative deposition), and molecular layer deposition (MLD).

Any step or component in the following functionalization process be omitted or changed in accordance with properties desired of the final functionalized substrate. In some cases, additional components and/or process steps are added to the process workflows embodied herein. In some instances, a substrate is first cleaned, for example, using a piranha solution. An example of a cleaning process includes soaking a substrate in a piranha solution (e.g., 90% H₂SO₄, 10% H₂O₂) at an elevated temperature (e.g., 120° C.) and washing (e.g., water) and drying the substrate (e.g., nitrogen gas). The process optionally includes a post piranha treatment comprising soaking the piranha treated substrate in a basic solution (e.g., NH₄OH) followed by an aqueous wash (e.g., water). In some instances, a surface of a structure is plasma cleaned, optionally following the piranha soak and optional post piranha treatment. An example of a plasma cleaning process comprises an oxygen plasma etch. In some instances, the surface is deposited with an active functionalization agent following by vaporization. In some instances, the substrate is actively functionalized prior to cleaning, for example, by piranha treatment and/or plasma cleaning.

The process for surface functionalization optionally comprises a resist coat and a resist strip. In some instances, following active surface functionalization, the substrate is spin coated with a resist, for example, SPR™ 3612 positive photoresist. The process for surface functionalization, in various instances, comprises lithography with patterned functionalization. In some instances, photolithography is performed following resist coating. In some instances, after lithography, the surface is visually inspected for lithography defects. The process for surface functionalization, in some instances, comprises a cleaning step, whereby residues of the substrate are removed, for example, by plasma cleaning or etching. In some instances, the plasma cleaning step is performed at some step after the lithography step.

In some instances, a surface coated with a resist is treated to remove the resist, for example, after functionalization and/or after lithography. In some cases, the resist is removed with a solvent, for example, with a stripping solution comprising N-methyl-2-pyrrolidone. In some cases, resist stripping comprises sonication or ultrasonication. In some instances, a resist is coated and stripped, followed by active functionalization of the exposed areas to create a desired differential functionalization pattern.

In some instances, the methods and compositions described herein relate to the application of photoresist for the generation of modified surface properties in selective areas, wherein the application of the photoresist relies on the fluidic properties of the surface defining the spatial distribution of the photoresist. Without being bound by theory, surface tension effects related to the applied fluid may define the flow of the photoresist. For example, surface tension and/or capillary action effects may facilitate drawing of the photoresist into small structures in a controlled fashion before the resist solvents evaporate. In some instances, resist contact points are pinned by sharp edges, thereby controlling the advance of the fluid. The underlying structures may be designed based on the desired flow patterns that are used to apply photoresist during the manufacturing and functionalization processes. A solid organic layer left behind after solvents evaporate may be used to pursue the subsequent steps of the manufacturing process. Structures may be designed to control the flow of fluids by facilitating or inhibiting wicking effects into neighboring fluidic paths. For example, a structure is designed to avoid overlap between top and bottom edges, which facilitates the keeping of the fluid in top structures allowing for a particular disposition of the resist. In an alternative example, the top and bottom edges overlap, leading to the wicking of the applied fluid into bottom structures. Appropriate designs may be selected accordingly, depending on the desired application of the resist.

In some instances, a structure described herein has a surface that comprises a material having thickness of at least or at least 0.1 nm, 0.5 nm, 1 nm, 2 nm, 5 nm, 10 nm or 25 nm that comprises a reactive group capable of binding nucleosides. Exemplary include, without limitation, glass and silicon, such as silicon dioxide and silicon nitride. In some cases, exemplary surfaces include nylon and PMMA.

In some instances, electromagnetic radiation in the form of UV light is used for surface patterning. In some instances, a lamp is used for surface patterning, and a mask mediates exposure locations of the UV light to the surface. In some instances, a laser is used for surface patterning, and a shutter opened/closed state controls exposure of the UV light to the surface. The laser arrangement may be used in combination with a flexible structure that is capable of moving. In such an arrangement, the coordination of laser exposure and flexible structure movement is used to create patterns of one or more agents having differing nucleoside coupling capabilities.

Described herein are surfaces for polynucleotide synthesis that are reusable. After synthesis and/or cleavage of polynucleotides, a surface may be bathed, washed, cleaned, baked, etched, or otherwise functionally restored to a condition suitable for subsequent polynucleotide synthesis. The number of times a surface is reused and the methods for recycling/preparing the surface for reuse vary depending on subsequent applications. Surfaces prepared for reuse are in some instances reused at least 1, 2, 3, 5, 10, 20, 50, 100, 1,000 or more times. In some instances, the remaining “life” or number of times a surface is suitable for reuse is measured or predicted.

### Material Deposition Systems

In some cases, the synthesized polynucleotides are stored on the substrate, for example a solid support. Nucleic acid reagents may be deposited on the substrate surface in a non-continuous, or drop-on-demand method. Examples of such methods include the electromechanical transfer method, electric thermal transfer method, and electrostatic attraction method. In the electromechanical transfer method, piezoelectric elements deformed by electrical pulses cause the droplets to be ejected. In the electric thermal transfer method, bubbles are generated in a chamber of the device, and the expansive force of the bubbles causes the droplets to be ejected. In the electrostatic attraction method, electrostatic force of attraction is used to eject the droplets onto the substrate. In some cases, the drop frequency is from about 5 KHz to about 500 KHz; from about 5 KHz to about 100 KHz; from about 10 KHz to about 500 KHz; from about 10 KHz to about 100 KHz; or from about 50 KHz to about 500 KHz. In some cases, the frequency is less than about 500 KHz, 200 KHz, 100 KHz, or 50 KHz.

The size of the droplets dispensed correlates to the resolution of the device. In some instances, the devices deposit droplets of reagents at sizes from about 0.01 pl to about 20 pl, from about 0.01 pl to about 10 pl, from about 0.01 pl to about 1 pl, from about 0.01 pl to about 0.5 pl, from about 0.01 pl to about 0.01 pl, or from about 0.05 pl to about 1 pl. In some instances, the droplet size is less than about 1 pl, 0.5 pl, 0.2 pl, 0.1 pl, or 0.05 pl.

In some arrangements, the configuration of a polynucleotide synthesis system allows for a continuous polynucleotide synthesis process that exploits the flexibility of a substrate for traveling in a reel-to-reel type process. This synthesis process operates in a continuous production line manner with the substrate travelling through various stages of polynucleotide synthesis using one or more reels to rotate the position of the substrate. In an exemplary instance, a polynucleotide synthesis reaction comprises rolling a substrate: through a solvent bath, beneath a deposition device for phosphoramidite deposition, through a bath of oxidizing agent, through an acetonitrile wash bath, and through a deblock bath. Optionally, the tape is also traversed through a capping bath. A reel-to-reel type process allows for the finished product of a substrate comprising synthesized polynucleotides to be easily gathered on a take-up reel, where it can be transported for further processing or storage.

In some arrangements, polynucleotide synthesis proceeds in a continuous process as a continuous flexible tape is conveyed along a conveyor belt system. Similar to the reel-to-reel type process, polynucleotide synthesis on a continuous tape operates in a production line manner, with the substrate travelling through various stages of polynucleotide synthesis during conveyance. However, in a conveyor belt process, the continuous tape revisits a polynucleotide synthesis step without rolling and unrolling of the tape, as in a reel-to-reel process. In some arrangements, polynucleotide synthesis steps are partitioned into zones and a continuous tape is conveyed through each zone one or more times in a cycle. For example, a polynucleotide synthesis reaction may comprise (1) conveying a substrate through a solvent bath, beneath a deposition device for phosphoramidite deposition, through a bath of oxidizing agent, through an acetonitrile wash bath, and through a block bath in a cycle; and then (2) repeating the cycles to achieve synthesized polynucleotides of a predetermined length. After polynucleotide synthesis, the flexible substrate is removed from the conveyor belt system and, optionally, rolled for storage. Rolling may be around a reel, for storage. In some instances, a flexible substrate comprising thermoplastic material is coated with nucleoside coupling reagent. The coating is patterned into loci such that each locus has diameter of about 10 um, with a center-to-center distance between two adjacent loci of about 21 μm. In this instance, the locus size is sufficient to accommodate a sessile drop volume of 0.2 pl during a polynucleotide synthesis deposition step. In some cases, the locus density is about 2.2 billion loci per m² (1 locus/441×10⁻¹² m²). In some cases, a 4.5 m² substrate comprise about 10 billion loci, each with a 10 um diameter.

In some arrangements, a device for application of one or more reagents to a substrate during a synthesis reaction is configured to deposit reagents and/or nucleoside monomers for nucleoside phosphoramidite based synthesis. Reagents for polynucleotide synthesis include reagents for polynucleotide extension and wash buffers. As non-limiting examples, the device deposits cleaning reagents, coupling reagents, capping reagents, oxidizers, de-blocking agents, acetonitrile, gases such as nitrogen gas, and any combination thereof. In addition, the device optionally deposits reagents for the preparation and/or maintenance of substrate integrity. In some instances, the polynucleotide synthesizer deposits a drop having a diameter less than about 200 μm, 100 um, or 50 μm in a volume less than about 1000, 500, 100, 50, or 20 pl. In some cases, the polynucleotide synthesizer deposits between about 1 and 10000, 1 and 5000, 100 and 5000, or 1000 and 5000 droplets per second.

Described herein are devices, methods, systems and compositions where reagents for polynucleotide synthesis are recycled or reused. Recycling of reagents may comprise collection, storage, and usage of unused reagents, or purification/transformation of used reagents. For example, a reagent bath is recycled and used for a polynucleotide synthesis step on the same or a different surface. Reagents described herein may be recycled 1, 2, 3, 4, 5, 6, 7, 8, 9, 10 or more times. Alternatively or in combination, a reagent solution comprising a reaction byproduct is filtered to remove the byproduct, and the reagent solution is used for additional polynucleotide synthesis reactions.

Many integrated or non-integrated elements are often used with polynucleotide synthesis systems. In some instances, a polynucleotide synthesis system comprises one or more elements useful for downstream processing of synthesized polynucleotides. As an example, the system comprises a temperature control element such as a thermal cycling device. In some instances, the temperature control element is used with a plurality of resolved reactors to perform nucleic acid assembly such as PCA and/or nucleic acid amplification such as PCR.

### De Novo Polynucleotide Synthesis

Provided herein are systems and methods for synthesis of a high density of polynucleotides on a substrate in a short amount of time. In some instances, the substrate is a flexible substrate. In some instances, at least 10¹⁰, 10¹¹, 10¹², 10¹³, 10¹⁴, or 10¹⁵ bases are synthesized in one day. In some instances, at least 10×10⁸, 10×10⁹, 10×10¹⁰, 10×10¹¹, or 10×10¹² polynucleotides are synthesized in one day. In some cases, each polynucleotide synthesized comprises at least 20, 25, 50, 100, 200, 300, 400 or at least 500 nucleobases. In some cases, each polynucleotide synthesized comprises 20-500, 25-500, 50-500, 100-500, 200-500, 300-500, 400-500 50-250, 50-300, 100-300, or 150-400 bases. In some cases, these bases are synthesized with a total average error rate of less than about 1 in 100; 200; 300; 400; 500; 1000; 2000; 5000; 10000; 15000; 20000 bases. In some instances, these error rates are for at least 50%, 60%, 70%, 80%, 90%, 95%, 98%, 99%, 99.5%, or more of the polynucleotides synthesized. In some instances, these at least 90%, 95%, 98%, 99%, 99.5%, or more of the polynucleotides synthesized do not differ from a predetermined sequence for which they encode. In some instances, the error rate for synthesized polynucleotides on a substrate using the methods and systems described herein is less than about 1 in 200. In some instances, the error rate for synthesized polynucleotides on a substrate using the methods and systems described herein is less than about 1 in 1,000. In some instances, the error rate for synthesized polynucleotides on a substrate using the methods and systems described herein is less than about 1 in 2,000. In some instances, the error rate for synthesized polynucleotides on a substrate using the methods and systems described herein is less than about 1 in 3,000. In some instances, the error rate for synthesized polynucleotides on a substrate using the methods and systems described herein is less than about 1 in 5,000. Individual types of error rates include mismatches, deletions, insertions, and/or substitutions for the polynucleotides synthesized on the substrate. The term “error rate” refers to a comparison of the collective amount of synthesized polynucleotide to an aggregate of predetermined polynucleotide sequences. In some instances, synthesized polynucleotides disclosed herein comprise a tether of 12 to 25 bases. In some instances, the tether comprises 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more bases.

Described herein are methods, systems, devices, and compositions wherein chemical reactions used in polynucleotide synthesis are controlled using electrochemistry. Electrochemical reactions in some instances are controlled by any source of energy, such as light, heat, radiation, or electricity. For example, electrodes are used to control chemical reactions as all or a portion of discrete loci on a surface. Electrodes in some instances are charged by applying an electrical potential to the electrode to control one or more chemical steps in polynucleotide synthesis. In some instances, these electrodes are addressable. Any number of the chemical steps described herein is in some instances controlled with one or more electrodes. Electrochemical reactions may comprise oxidations, reductions, acid/base chemistry, or other reaction that is controlled by an electrode. In some instances, electrodes generate electrons or protons that are used as reagents for chemical transformations. Electrodes in some instances directly generate a reagent such as an acid. In some instances, an acid is a proton. Electrodes in some instances directly generate a reagent such as a base. Acids or bases are often used to cleave protecting groups, or influence the kinetics of various polynucleotide synthesis reactions, for example by adjusting the pH of a reaction solution. Electrochemically controlled polynucleotide synthesis reactions in some instances comprise redox-active metals or other redox-active organic materials. In some instances, metal or organic catalysts are employed with these electrochemical reactions. In some instances, acids are generated from oxidation of quinones.

Control of chemical reactions with is not limited to the electrochemical generation of reagents; chemical reactivity may be influenced indirectly through biophysical changes to substrates or reagents through electric fields (or gradients) which are generated by electrodes. In some instances, substrates include but are not limited to nucleic acids. In some instances, electrical fields which repel or attract specific reagents or substrates towards or away from an electrode or surface are generated. Such fields in some instances are generated by application of an electrical potential to one or more electrodes. For example, negatively charged nucleic acids are repelled from negatively charged electrode surfaces. Such repulsions or attractions of polynucleotides or other reagents caused by local electric fields in some instances provides for movement of polynucleotides or other reagents in or out of region of the synthesis device or structure. In some instances, electrodes generate electric fields which repel polynucleotides away from a synthesis surface, structure, or device. In some instances, electrodes generate electric fields which attract polynucleotides towards a synthesis surface, structure, or device. In some instances, protons are repelled from a positively charged surface to limit contact of protons with substrates or portions thereof. In some instances, repulsion or attractive forces are used to allow or block entry of reagents or substrates to specific areas of the synthesis surface. In some instances, nucleoside monomers are prevented from contacting a polynucleotide chain by application of an electric field in the vicinity of one or both components. Such arrangements allow gating of specific reagents, which may obviate the need for protecting groups when the concentration or rate of contact between reagents and/or substrates is controlled. In some instances, unprotected nucleoside monomers are used for polynucleotide synthesis. Alternatively, application of the field in the vicinity of one or both components promotes contact of nucleoside monomers with a polynucleotide chain. Additionally, application of electric fields to a substrate can alter the substrates reactivity or conformation. In an exemplary application, electric fields generated by electrodes are used to prevent polynucleotides at adjacent loci from interacting. In some instances, the substrate is a polynucleotide, optionally attached to a surface. Application of an electric field in some instances alters the three-dimensional structure of a polynucleotide. Such alterations comprise folding or unfolding of various structures, such as helices, hairpins, loops, or other 3-dimensional nucleic acid structure. Such alterations are useful for manipulating nucleic acids inside of wells, channels, or other structures. In some instances, electric fields are applied to a nucleic acid substrate to prevent secondary structures. In some instances, electric fields obviate the need for linkers or attachment to a solid support during polynucleotide synthesis.

A suitable method for polynucleotide synthesis on a substrate of this disclosure is a phosphoramidite method comprising the controlled addition of a phosphoramidite building block, i.e. nucleoside phosphoramidite, to a growing polynucleotide chain in a coupling step that forms a phosphite triester linkage between the phosphoramidite building block and a nucleoside bound to the substrate. In some instances, the nucleoside phosphoramidite is provided to the substrate activated. In some instances, the nucleoside phosphoramidite is provided to the substrate with an activator. In some instances, nucleoside phosphoramidites are provided to the substrate in a 1.5, 2, 3, 4, 5, 6, 7, 8, 9, 10, 11, 12, 13, 14, 15, 16, 17, 18, 19, 20, 25, 30, 35, 40, 50, 60, 70, 80, 90, 100-fold excess or more over the substrate-bound nucleosides. In some instances, the addition of nucleoside phosphoramidite is performed in an anhydrous environment, for example, in anhydrous acetonitrile. Following addition and linkage of a nucleoside phosphoramidite in the coupling step, the substrate is optionally washed. In some instances, the coupling step is repeated one or more additional times, optionally with a wash step between nucleoside phosphoramidite additions to the substrate. In some instances, a polynucleotide synthesis method used herein comprises 1, 2, 3 or more sequential coupling steps. Prior to coupling, in many cases, the nucleoside bound to the substrate is de-protected by removal of a protecting group, where the protecting group functions to prevent polymerization. Protecting groups may comprise any chemical group that prevents extension of the polynucleotide chain. In some instances, the protecting group is cleaved (or removed) in the presence of an acid. In some instances, the protecting group is cleaved in the presence of a base. In some instances, the protecting group is removed with electromagnetic radiation such as light, heat, or other energy source. In some instances, the protecting group is removed through an oxidation or reduction reaction. In some instances, a protecting group comprises a triarylmethyl group. In some instances, a protecting group comprises an aryl ether. In some instances, a protecting comprises a disulfide. In some instances a protecting group comprises an acid-labile silane. In some instances, a protecting group comprises an acetal. In some instances, a protecting group comprises a ketal. In some instances, a protecting group comprises an enol ether. In some instances, a protecting group comprises a methoxybenzyl group. In some instances, a protecting group comprises an azide. In some instances, a protecting group is 4,4′-dimethoxytrityl (DMT). In some instances, a protecting group is a tert-butyl carbonate. In some instances, a protecting group is a tert-butyl ester. In some instances, a protecting group comprises a base-labile group.

Following coupling, phosphoramidite polynucleotide synthesis methods optionally comprise a capping step. In a capping step, the growing polynucleotide is treated with a capping agent. A capping step generally serves to block unreacted substrate-bound 5′-OH groups after coupling from further chain elongation, preventing the formation of polynucleotides with internal base deletions. Further, phosphoramidites activated with 1H-tetrazole often react, to a small extent, with the 06 position of guanosine. Without being bound by theory, upon oxidation with I₂/water, this side product, possibly via O6-N7 migration, undergoes depurination. The apurinic sites can end up being cleaved in the course of the final deprotection of the polynucleotide thus reducing the yield of the full-length product. The O6 modifications may be removed by treatment with the capping reagent prior to oxidation with I₂/water. In some instances, inclusion of a capping step during polynucleotide synthesis decreases the error rate as compared to synthesis without capping. As an example, the capping step comprises treating the substrate-bound polynucleotide with a mixture of acetic anhydride and 1-methylimidazole. Following a capping step, the substrate is optionally washed.

Following addition of a nucleoside phosphoramidite, and optionally after capping and one or more wash steps, a substrate described herein comprises a bound growing nucleic acid that may be oxidized. The oxidation step comprises oxidizing the phosphite triester into a tetracoordinated phosphate triester, a protected precursor of the naturally occurring phosphate diester internucleoside linkage. In some instances, phosphite triesters are oxidized electrochemically. In some instances, oxidation of the growing polynucleotide is achieved by treatment with iodine and water, optionally in the presence of a weak base such as a pyridine, lutidine, or collidine. Oxidation is sometimes carried out under anhydrous conditions using tert-Butyl hydroperoxide or (1S)-(+)-(10-camphorsulfonyl)-oxaziridine (CSO). In some methods, a capping step is performed following oxidation. A second capping step allows for substrate drying, as residual water from oxidation that may persist can inhibit subsequent coupling. Following oxidation, the substrate and growing polynucleotide is optionally washed. In some instances, the step of oxidation is substituted with a sulfurization step to obtain polynucleotide phosphorothioates, wherein any capping steps can be performed after the sulfurization. Many reagents are capable of the efficient sulfur transfer, including, but not limited to, 3-(Dimethylaminomethylidene)amino)-3H-1,2,4-dithiazole-3-thione, DDTT, 3H-1,2-benzodithiol-3-one 1,1-dioxide, also known as Beaucage reagent, and N,N,N′N′-Tetraethylthiuram disulfide (TETD).

For a subsequent cycle of nucleoside incorporation to occur through coupling, a protected 5′ end (or 3′ end, if synthesis is conducted in a 5′ to 3′ direction) of the substrate bound growing polynucleotide is be removed so that the primary hydroxyl group can react with a next nucleoside phosphoramidite. In some instances, the protecting group is DMT and deblocking occurs with trichloroacetic acid in dichloromethane. In some instances, the protecting group is DMT and deblocking occurs with electrochemically generated protons. Conducting detritylation for an extended time or with stronger than recommended solutions of acids may lead to increased depurination of solid support-bound polynucleotide and thus reduces the yield of the desired full-length product. Methods and compositions described herein provide for controlled deblocking conditions limiting undesired depurination reactions. In some instances, the substrate bound polynucleotide is washed after deblocking. In some cases, efficient washing after deblocking contributes to synthesized polynucleotides having a low error rate.

Methods for the synthesis of polynucleotides on a substrate described herein may involve an iterating sequence of the following steps: application of a protected monomer to a surface of a substrate feature to link with either the surface, a linker or with a previously deprotected monomer; deprotection of the applied monomer so that it can react with a subsequently applied protected monomer; and application of another protected monomer for linking. One or more intermediate steps include oxidation and/or sulfurization. In some instances, one or more wash steps precede or follow one or all of the steps.

Methods for the synthesis of polynucleotides on a substrate described herein may comprise an oxidation step. For example, methods involve an iterating sequence of the following steps: application of a protected monomer to a surface of a substrate feature to link with either the surface, a linker or with a previously deprotected monomer; deprotection of the applied monomer so that it can react with a subsequently applied protected monomer; application of another protected monomer for linking, and oxidation and/or sulfurization. In some instances, one or more wash steps precede or follow one or all of the steps.

Methods for the synthesis of polynucleotides on a substrate described herein may further comprise an iterating sequence of the following steps: application of a protected monomer to a surface of a substrate feature to link with either the surface, a linker or with a previously deprotected monomer; deprotection of the applied monomer so that it can react with a subsequently applied protected monomer; and oxidation and/or sulfurization. In some instances, one or more wash steps precede or follow one or all of the steps.

Methods for the synthesis of polynucleotides on a substrate described herein may further comprise an iterating sequence of the following steps: application of a protected monomer to a surface of a substrate feature to link with either the surface, a linker or with a previously deprotected monomer; and oxidation and/or sulfurization. In some instances, one or more wash steps precede or follow one or all of the steps.

Methods for the synthesis of polynucleotides on a substrate described herein may further comprise an iterating sequence of the following steps: application of a protected monomer to a surface of a substrate feature to link with either the surface, a linker or with a previously deprotected monomer; deprotection of the applied monomer so that it can react with a subsequently applied protected monomer; and oxidation and/or sulfurization. In some instances, one or more wash steps precede or follow one or all of the steps.

In some instances, polynucleotides are synthesized with photolabile protecting groups, where the hydroxyl groups generated on the surface are blocked by photolabile-protecting groups. When the surface is exposed to UV light, such as through a photolithographic mask, a pattern of free hydroxyl groups on the surface may be generated. These hydroxyl groups can react with photoprotected nucleoside phosphoramidites, according to phosphoramidite chemistry. A second photolithographic mask can be applied and the surface can be exposed to UV light to generate second pattern of hydroxyl groups, followed by coupling with 5′-photoprotected nucleoside phosphoramidite. Likewise, patterns can be generated and oligomer chains can be extended. Without being bound by theory, the lability of a photocleavable group depends on the wavelength and polarity of a solvent employed and the rate of photocleavage may be affected by the duration of exposure and the intensity of light. This method can leverage a number of factors such as accuracy in alignment of the masks, efficiency of removal of photo-protecting groups, and the yields of the phosphoramidite coupling step. Further, unintended leakage of light into neighboring sites can be minimized. The density of synthesized oligomer per spot can be monitored by adjusting loading of the leader nucleoside on the surface of synthesis.

The surface of a substrate described herein that provides support for polynucleotide synthesis may be chemically modified to allow for the synthesized polynucleotide chain to be cleaved from the surface. In some instances, the polynucleotide chain is cleaved at the same time as the polynucleotide is deprotected. In some cases, the polynucleotide chain is cleaved after the polynucleotide is deprotected. In an exemplary scheme, a trialkoxysilyl amine such as (CH₃CH₂O)₃Si—(CH₂)₂—NH₂ is reacted with surface SiOH groups of a substrate, followed by reaction with succinic anhydride with the amine to create an amide linkage and a free OH on which the nucleic acid chain growth is supported. Cleavage includes gas cleavage with ammonia or methylamine. In some instances cleavage includes linker cleavage with electrically generated reagents such as acids or bases. In some instances, once released from the surface, polynucleotides are assembled into larger nucleic acids that are sequenced and decoded to extract stored information.

The surfaces described herein can be reused after polynucleotide cleavage to support additional cycles of polynucleotide synthesis. For example, the linker can be reused without additional treatment/chemical modifications. In some instances, a linker is non-covalently bound to a substrate surface or a polynucleotide. In some embodiments, the linker remains attached to the polynucleotide after cleavage from the surface. Linkers in some embodiments comprise reversible covalent bonds such as esters, amides, ketals, beta substituted ketones, heterocycles, or other group that is capable of being reversibly cleaved. Such reversible cleavage reactions are in some instances controlled through the addition or removal of reagents, or by electrochemical processes controlled by electrodes. Optionally, chemical linkers or surface-bound chemical groups are regenerated after a number of cycles, to restore reactivity and remove unwanted side product formation on such linkers or surface-bound chemical groups.

### Assembly

Polynucleotides may be designed to collectively span a large region of a predetermined sequence that encodes for information. In some instances, larger polynucleotides are generated through ligation reactions to join the synthesized polynucleotides. One example of a ligation reaction is polymerase chain assembly (PCA). In some instances, at least of a portion of the polynucleotides are designed to include an appended region that is a substrate for universal primer binding. For PCA reactions, the presynthesized polynucleotides include overlaps with each other (e.g., 4, 20, 40 or more bases with overlapping sequence). During the polymerase cycles, the polynucleotides anneal to complementary fragments and then are filled in by polymerase. Each cycle thus increases the length of various fragments randomly depending on which polynucleotides find each other. Complementarity amongst the fragments allows for forming a complete large span of double-stranded DNA. In some cases, after the PCA reaction is complete, an error correction step is conducted using mismatch repair detecting enzymes to remove mismatches in the sequence. Once larger fragments of a target sequence are generated, they can be amplified. For example, in some cases, a target sequence comprising 5′ and 3′ terminal adapter sequences is amplified in a polymerase chain reaction (PCR) which includes modified primers that hybridize to the adapter sequences. In some cases, the modified primers comprise one or more uracil bases. The use of modified primers allows for removal of the primers through enzymatic reactions centered on targeting the modified base and/or gaps left by enzymes which cleave the modified base pair from the fragment. What remains is a double-stranded amplification product that lacks remnants of adapter sequence. In this way, multiple amplification products can be generated in parallel with the same set of primers to generate different fragments of double-stranded DNA.

Error correction may be performed on synthesized polynucleotides and/or assembled products. An example strategy for error correction involves site-directed mutagenesis by overlap extension PCR to correct errors, which is optionally coupled with two or more rounds of cloning and sequencing. In certain instances, double-stranded nucleic acids with mismatches, bulges and small loops, chemically altered bases and/or other heteroduplexes are selectively removed from populations of correctly synthesized nucleic acids. In some instances, error correction is performed using proteins/enzymes that recognize and bind to or next to mismatched or unpaired bases within double-stranded nucleic acids to create a single or double-strand break or to initiate a strand transfer transposition event. Non-limiting examples of proteins/enzymes for error correction include endonucleases (T7 Endonuclease I, E. coli Endonuclease V, T4 Endonuclease VII, mung bean nuclease, Cell, E. coli Endonuclease IV, UVDE), restriction enzymes, glycosylases, ribonucleases, mismatch repair enzymes, resolvases, helicases, ligases, antibodies specific for mismatches, and their variants. Examples of specific error correction enzymes include T4 endonuclease 7, T7 endonuclease 1, S1, mung bean endonuclease, MutY, MutS, MutH, MutL, cleavase, CELI, and HINF1. In some cases, DNA mismatch-binding protein MutS (Thermus aquaticus) is used to remove failure products from a population of synthesized products. In some instances, error correction is performed using the enzyme Correctase. In some cases, error correction is performed using SURVEYOR endonuclease (Transgenomic), a mismatch-specific DNA endonuclease that scans for known and unknown mutations and polymorphisms for heteroduplex DNA.

### Sequencing

After extraction and/or amplification of polynucleotides from the surface of the structure, suitable sequencing technology may be employed to sequence the polynucleotides. In some cases, the DNA sequence is read on the substrate or within a feature of a structure. In some cases, the polynucleotides stored on the substrate are extracted is optionally assembled into longer nucleic acids and then sequenced.

Polynucleotides synthesized and stored on the structures described herein encode data that can be interpreted by reading the sequence of the synthesized polynucleotides and converting the sequence into binary code readable by a computer. In some cases the sequences require assembly, and the assembly step may need to be at the nucleic acid sequence stage or at the digital sequence stage.

Provided herein are detection systems comprising a device capable of sequencing stored polynucleotides, either directly on the structure and/or after removal from the main structure. In cases where the structure is a reel-to-reel tape of flexible material, the detection system comprises a device for holding and advancing the structure through a detection location and a detector disposed proximate the detection location for detecting a signal originated from a section of the tape when the section is at the detection location. In some instances, the signal is indicative of a presence of a polynucleotide. In some instances, the signal is indicative of a sequence of a polynucleotide (e.g., a fluorescent signal). In some instances, information encoded within polynucleotides on a continuous tape is read by a computer as the tape is conveyed continuously through a detector operably connected to the computer. In some instances, a detection system comprises a computer system comprising a polynucleotide sequencing device, a database for storage and retrieval of data relating to polynucleotide sequence, software for converting DNA code of a polynucleotide sequence to binary code, a computer for reading the binary code, or any combination thereof.

Provided herein are sequencing systems that can be integrated into the devices described herein. Various methods of sequencing are well known in the art, and comprise “base calling” wherein the identity of a base in the target polynucleotide is identified. In some instances, polynucleotides synthesized using the methods, devices, compositions, and systems described herein are sequenced after cleavage from the synthesis surface. In some instances, sequencing occurs during or simultaneously with polynucleotide synthesis, wherein base calling occurs immediately after or before extension of a nucleoside monomer into the growing polynucleotide chain. Methods for base calling include measurement of electrical currents/voltages generated by polymerase-catalyzed addition of bases to a template strand. In some instances, synthesis surfaces comprise enzymes, such as polymerases. In some instances, such enzymes are tethered to electrodes or to the synthesis surface.

### Computer Systems

In various aspects, any of the systems described herein are operably linked to a computer and are optionally automated through a computer either locally or remotely. In various instances, the methods and systems of the invention further comprise software programs on computer systems and use thereof. Accordingly, computerized control for the synchronization of the dispense/vacuum/refill functions such as orchestrating and synchronizing the material deposition device movement, dispense action and vacuum actuation are within the bounds of the invention. In some instances, the computer systems are programmed to interface between the user specified base sequence and the position of a material deposition device to deliver the correct reagents to specified regions of the substrate.

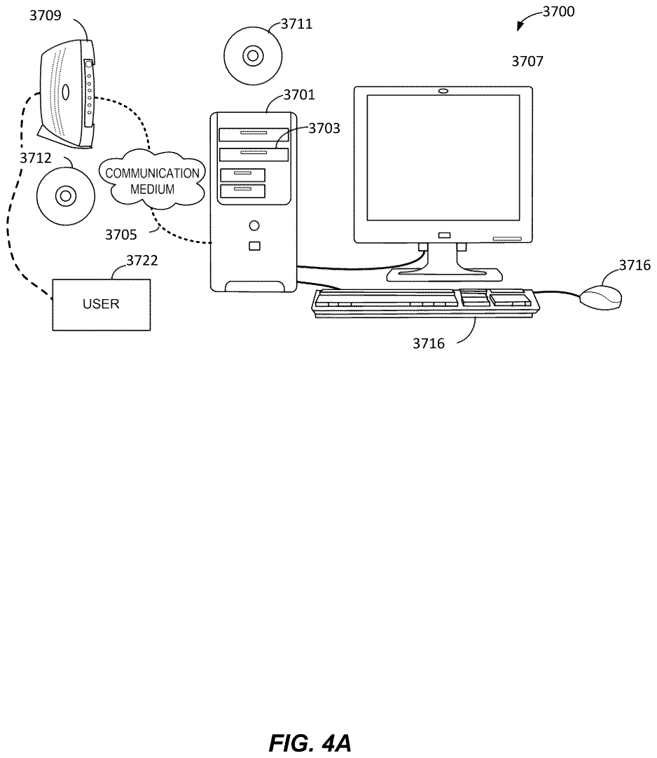

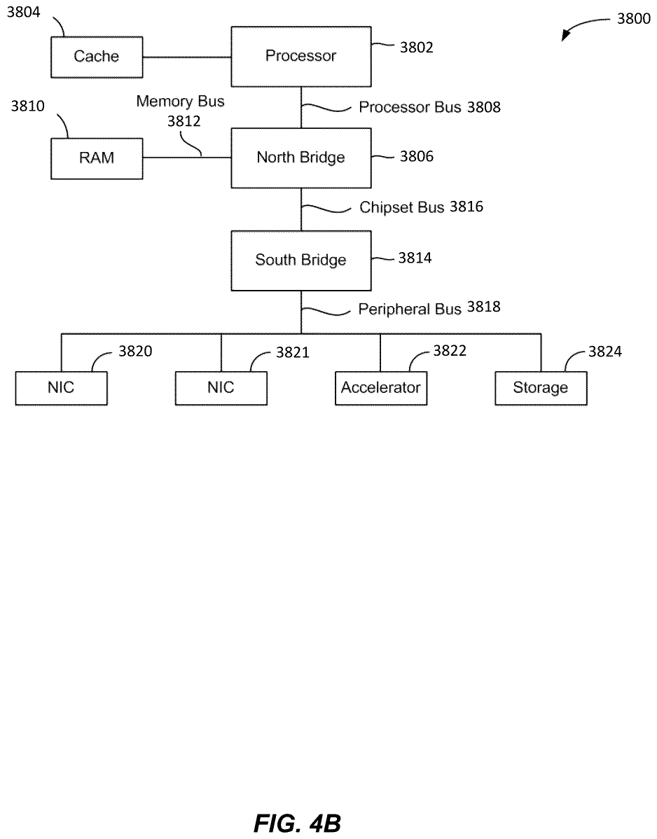

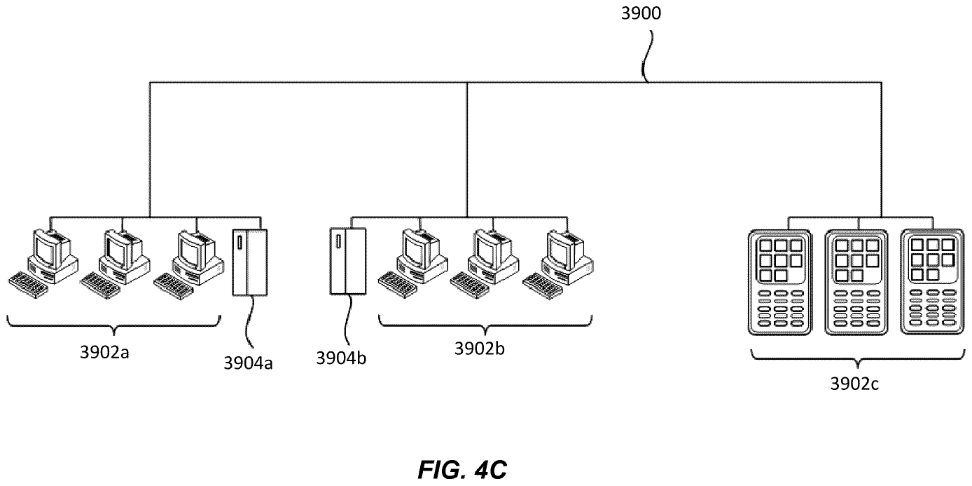

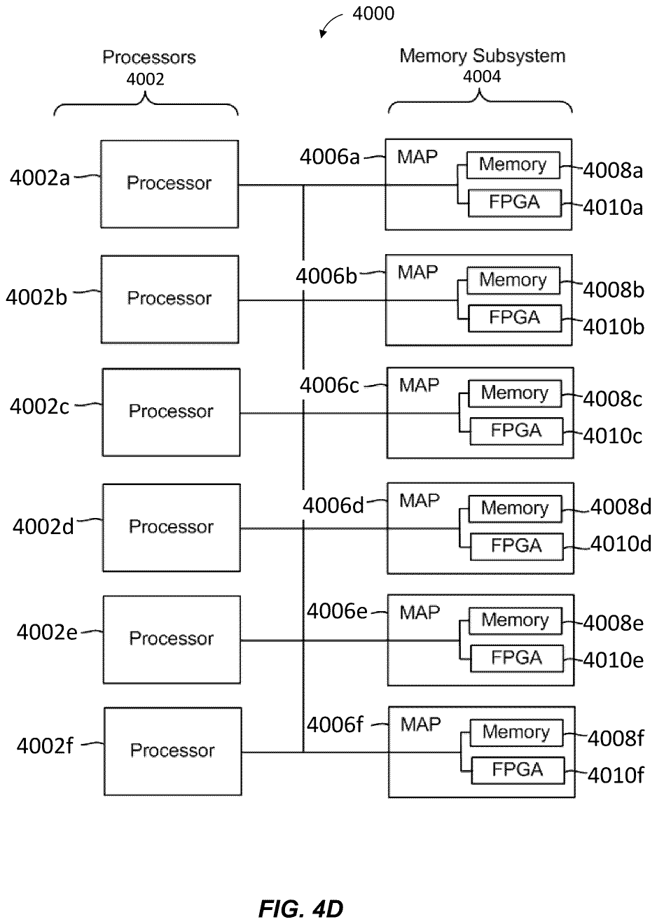

The computer system 3700 illustrated in FIG. 4A may be understood as a logical apparatus that can read instructions from media 3711 and/or a network port 3705, which can optionally be connected to server 3709 having fixed media 3712. The system can include a CPU 3701, disk drives 3703, optional input devices such as keyboard 3715 and/or mouse 3716 and optional monitor 3707. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 3722.

FIG. 4B is a block diagram illustrating a first example architecture of a computer system that can be used in connection with example instances of the present invention. As depicted in FIG. 5, the example computer system can include a processor 3802 for processing instructions. Non-limiting examples of processors include: Intel Xeon™ processor, AMD Opteron™ processor, Samsung 32-bit RISC ARM 1176JZ(F)—S v1.0™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some instances, multiple processors or processors with multiple cores can also be used, whether in a single computer system, in a cluster, or distributed across systems over a network comprising a plurality of computers, cell phones, and/or personal data assistant devices. As illustrated in FIG. 38, a high speed cache 3804 can be connected to, or incorporated in, the processor 3802 to provide a high speed memory for instructions or data that have been recently, or are frequently, used by processor 3802. The processor 3802 is connected to a north bridge 3806 by a processor bus 3808. The north bridge 3806 is connected to random access memory (RAM) 3810 by a memory bus 3812 and manages access to the RAM 3810 by the processor 3802. The north bridge 3806 is also connected to a south bridge 3814 by a chipset bus 3816. The south bridge 3814 is, in turn, connected to a peripheral bus 3818. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 3818. In some alternative architectures, the functionality of the north bridge can be incorporated into the processor instead of using a separate north bridge chip. In some instances, a system 3800 can include an accelerator card 3822 attached to the peripheral bus 3818. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 3824 and can be loaded into RAM 3810 and/or cache 3804 for use by the processor. The system 3800 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with example embodiments of the present invention. In this example, system 3800 also includes network interface cards (NICs) 3820 and 3821 connected to the peripheral bus for providing network interfaces to external storage, such as Network Attached Storage (NAS) and other computer systems that can be used for distributed parallel processing.

FIG. 4C is a diagram showing a network 3900 with a plurality of computer systems 3902a, and 3902b, a plurality of cell phones and personal data assistants 3902c, and Network Attached Storage (NAS) 3904a, and 3904b. In example embodiments, systems 3902a, 3902b, and 3902c can manage data storage and optimize data access for data stored in Network Attached Storage (NAS) 3904a and 3904b. A mathematical model can be used for the data and be evaluated using distributed parallel processing across computer systems 3902a, and 3902b, and cell phone and personal data assistant systems 3902c. Computer systems 3902a, and 3902b, and cell phone and personal data assistant systems 3902c can also provide parallel processing for adaptive data restructuring of the data stored in Network Attached Storage (NAS) 3904a and 3904b. FIG. 39 illustrates an example only, and a wide variety of other computer architectures and systems can be used in conjunction with the various embodiments of the present invention. For example, a blade server can be used to provide parallel processing. Processor blades can be connected through a back plane to provide parallel processing. Storage can also be connected to the back plane or as Network Attached Storage (NAS) through a separate network interface.

In some example embodiments, processors can maintain separate memory spaces and transmit data through network interfaces, back plane or other connectors for parallel processing by other processors. In other embodiments, some or all of the processors can use a shared virtual address memory space. FIG. 4D is a block diagram of a multiprocessor computer system 4000 using a shared virtual address memory space in accordance with an example embodiment. The system includes a plurality of processors 4002a-f that can access a shared memory subsystem 4004. The system incorporates a plurality of programmable hardware memory algorithm processors (MAPs) 4006a-f in the memory subsystem 4004. Each MAP 4006a-f can comprise a memory 4008a-f and one or more field programmable gate arrays (FPGAs) 4010a-f. The MAP provides a configurable functional unit and particular algorithms or portions of algorithms can be provided to the FPGAs 4010a-f for processing in close coordination with a respective processor. For example, the MAPs can be used to evaluate algebraic expressions regarding the data model and to perform adaptive data restructuring in example embodiments. In this example, each MAP is globally accessible by all of the processors for these purposes. In one configuration, each MAP can use Direct Memory Access (DMA) to access an associated memory 4008a-f, allowing it to execute tasks independently of, and asynchronously from, the respective microprocessor 4002a-f. In this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example embodiments, including systems using any combination of general processors, co-processors, FPGAs and other programmable logic devices, system on chips (SOCs), application specific integrated circuits (ASICs), and other processing and logic elements. In some embodiments, all or part of the computer system can be implemented in software or hardware. Any variety of data storage media can be used in connection with example embodiments, including random access memory, hard drives, flash memory, tape drives, disk arrays, Network Attached Storage (NAS) and other local or distributed data storage devices and systems.

In example embodiments, the computer system can be implemented using software modules executing on any of the above or other computer architectures and systems. In other embodiments, the functions of the system can be implemented partially or completely in firmware, programmable logic devices such as field programmable gate arrays (FPGAs), system on chips (SOCs), application specific integrated circuits (ASICs), or other processing and logic elements. For example, the Set Processor and Optimizer can be implemented with hardware acceleration through the use of a hardware accelerator card.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EMBODIMENTS

Provided herein are numbered embodiments 1-36. Embodiment 1. A composition for electrochemical acid generation comprising: a first redox compound; a second redox compound; an organic salt; and at least one solvent, wherein the redox potential between the first redox compound and the second redox compound is less than 2 volts in the solvent, and wherein application of a voltage to the composition results in electrochemical acid generation. Embodiment 2. The composition of embodiment 1, wherein the first redox compound or the second redox compound independently has the structure:

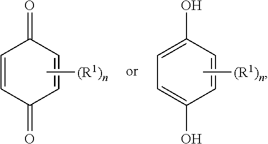

wherein each R¹ is independently hydrogen, halogen, —CN, —OR^{a}, —SR^{a}, —S(═O)R, —NO₂, —NR^{c}R^{d}, —S(═O)₂R^{d}, —NR^{a}S(═O)₂R^{d}, —S(═O)₂NR^{c}R^{d}, —C(═O)R, —OC(═O)R^{b}, —CO₂R^{a}, —OCO₂R^{a}, —C(═O)NR^{c}R^{d}, —OC(═O)NR^{c}R^{d}, —NR^{a}C(═O)NR^{c}R^{d}, —NR^{a}C(═O)R, —NR^{a}C(═O)OR^{a}, C₁-C₆ alkyl, C₂-C₆ alkenyl, C₂-C₆ alkynyl, C₁-C₆ heteroalkyl, C₃-C₈ cycloalkyl, C₂-C₈ heterocycloalkyl, aryl, or heteroaryl; wherein the alkyl, alkenyl, alkynyl, and heteroalkyl is optionally substituted with one, two, or three of halogen or —OR^{a}; and the cycloalkyl, heterocycloalkyl, aryl, and heteroaryl is optionally substituted with one, two, or three of halogen, C₁-C₆ alkyl, C₁-C₆ haloalkyl, —OR^{a}, or —NR^{c}R^{d}; or two or more R¹ are taken together to form a cycloalkyl, heterocycloalkyl, aryl, or heteroaryl ring; R^{a} is hydrogen, C₁-C₆ alkyl, C₂-C₆ alkenyl, C₂-C₆ alkynyl, C₁-C₆ heteroalkyl, C₃-C₈ cycloalkyl, C₂-C₈ heterocycloalkyl, aryl, or heteroaryl; wherein the alkyl, alkenyl, alkynyl, and heteroalkyl is optionally substituted with one, two, or three of halogen, —OH, —OMe, or —NH₂; and the cycloalkyl, heterocycloalkyl, aryl, and heteroaryl is optionally substituted with one, two, or three of halogen, C₁-C₆ alkyl, C₁-C₆ haloalkyl, —OH, —OMe, or —NH₂; R is C₁-C₆ alkyl, C₂-C₆ alkenyl, C₂-C₆ alkynyl, C₁-C₆ heteroalkyl, C₃-C₈ cycloalkyl, C₂-C₈ heterocycloalkyl, aryl, or heteroaryl; wherein the alkyl, alkenyl, alkynyl, and heteroalkyl is optionally substituted with one, two, or three of halogen, —OH, —OMe, or —NH₂; and the cycloalkyl, heterocycloalkyl, aryl, and heteroaryl is optionally substituted with one, two, or three of halogen, C₁-C₆ alkyl, C₁-C₆ haloalkyl, —OH, —OMe, or —NH₂; each R^{c} and R^{d} is independently hydrogen, C₁-C₆ alkyl, C₂-C₆ alkenyl, C₂-C₆ alkynyl, C₁-C₆ heteroalkyl, C₃-C₈ cycloalkyl, C₂-C₈ heterocycloalkyl, aryl, or heteroaryl; wherein the alkyl, alkenyl, alkynyl, and heteroalkyl is optionally substituted with one, two, or three of halogen, —OH, —OMe, or —NH₂; and the cycloalkyl, heterocycloalkyl, aryl, and heteroaryl is optionally substituted with one, two, or three of halogen, C₁-C₆ alkyl, C₁-C₆ haloalkyl, —OH, —OMe, or —NH₂; or R^{c} and R^{d}, together with the nitrogen atom to which they are attached, form a heterocycloalkyl or heteroaryl; wherein the heterocycloalkyl and heteroaryl is optionally substituted with one, two, or three of halogen, C₁-C₆ alkyl, C₁-C₆ haloalkyl, —OH, —OMe, or —NH₂; and n is 0-4. Embodiment 3. The composition of any one of embodiments 1-2, wherein the first redox compound has the structure:

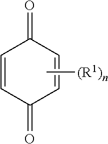

and the second redox compound has the structure:

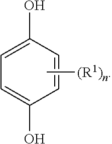

Embodiment 4. The composition of embodiment 3, wherein the first redox compound has the structure:

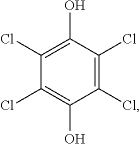 [SMILES: Oc1c(Cl)c(Cl)c(O)c(Cl)c1Cl]

and the second redox compound has the structure

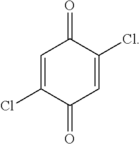

Embodiment 5. The composition of embodiment 3, wherein the first redox compound has the structure:

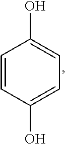

and the second redox compound has the structure

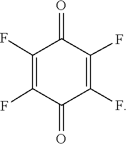 [SMILES: O=C1C(F)=C(F)C(=O)C(F)=C1F]

Embodiment 6. The composition of embodiment 3, wherein the first redox compound has the structure:

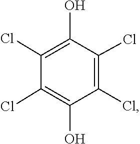 [SMILES: Oc1c(Cl)c(Cl)c(O)c(Cl)c1Cl]

and the second redox compound has the structure

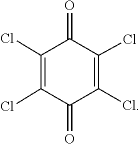 [SMILES: O=C1C(Cl)=C(Cl)C(=O)C(Cl)=C1Cl]

Embodiment 7. The composition of embodiment 3, wherein the first redox compound has the structure:

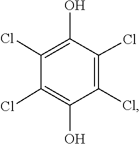 [SMILES: Oc1c(Cl)c(Cl)c(O)c(Cl)c1Cl]

and the second redox compound has the structure

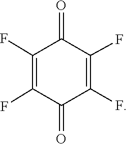 [SMILES: O=C1C(F)=C(F)C(=O)C(F)=C1F]

Embodiment 8. The composition of any one of embodiments 1-7, wherein the organic salt comprises a tetraalkylammonium cation. Embodiment 9. The composition of any one of embodiments 1-8, wherein the organic salt comprises a hexafluorophosphate anion. Embodiment 10. The composition of embodiment 1-7, wherein the organic salt is tetrabutylammonium hexafluorophosphate. Embodiment 11. The composition of embodiment 1-10, wherein the at least one solvent is acetonitrile, methanol, ethanol, dichloromethane, chloroform, 1,2-dichloromethane, dimethylformamide, ethylene glycol, propylene carbonate, or a mixture thereof. Embodiment 12. The composition of embodiment 1-11, wherein the concentration of the first redox compound and the concentration of the second redox compound is 0.1-2M. Embodiment 13. The composition of embodiment 1-11, wherein the concentration of the first redox compound and the concentration of the second redox compound is 0.1-0.5M. Embodiment 14. The composition of embodiment 1-13, wherein the concentration of the organic salt is 10-50 mM. Embodiment 15. The composition of embodiment 1-14, wherein the composition does not comprise an amine base. Embodiment 16. The composition of embodiment 15, wherein the amine base is non-nucleophilic base. Embodiment 17. The composition of embodiment 15, wherein the amine base is 2,6-lutidine, DIPEA, DBU, or pyridine. Embodiment 18. A method for polynucleotide synthesis comprising: contacting a nucleoside attached to a solid support with a protected nucleoside, wherein the protected nucleoside is configured to form a covalent bond with the nucleoside to generate a protected polynucleotide; contacting the protected polynucleotide with a composition of any one of embodiments 1-16, and applying a voltage to a solvent in fluid communication with the protected polynucleotide, wherein the voltage results in deprotection of the terminal nucleoside of the protected polynucleotide. Embodiment 19. The method of embodiment 18, wherein the voltage is less than 2 volts. Embodiment 20. The method of embodiment 18, wherein the voltage is 0.1-2 volts. Embodiment 21. The method of embodiment 19, wherein the voltage is applied for 0.001-5000 seconds. Embodiment 22. The method of embodiment 19, wherein the voltage is applied for 0.001-5 seconds. Embodiment 23. The method of any one of embodiments 18-22, wherein the voltage is applied in one or more pulses. Embodiment 24. The method of embodiment 23, wherein the time between pulses is 0-500 milliseconds. Embodiment 25. The method of any one of embodiments 18-24, wherein the protected polynucleotide comprises an acid-cleavable protecting group. Embodiment 26. The method of embodiment 18-25, wherein the voltage generates acid. Embodiment 27. The method of any one of embodiments 18-26, wherein the protected polynucleotide is 25-500 bases in length. Embodiment 28. A device for polynucleotide synthesis comprising: a surface comprising a plurality of loci configured for polynucleotide synthesis, wherein the composition of embodiments 1-17 is in contact with one or more loci; and a plurality of vias and/or routing configured for addressable control of the plurality of loci, wherein the area of each loci is 50-500 nm. Embodiment 29. The device of embodiment 28, wherein the loci comprises a pitch distance of no more than 1000 nm. Embodiment 30. The device of embodiment 28, wherein the loci comprises a pitch distance of no more than 500 nm. Embodiment 31. The device of embodiment 28, wherein the loci comprises a pitch distance of no more than 250 nm. Embodiment 32. The device of embodiment 28, wherein the loci comprises a pitch distance of no more than 100 nm. Embodiment 33. The device of any one of embodiments 28-32, wherein the device comprises at least 10 loci per square micron. Embodiment 34. The device of any one of embodiments 28-32, wherein the device comprises at least 5 loci per square micron. Embodiment 35. The device of any one of embodiments 28-34, wherein the device is integrated into a CMOS. Embodiment 36. The device of any one of embodiments 28-35, wherein the device further comprises a fluidics interface.

### EXAMPLES

### Example 1: Functionalization of a Device Surface

A device was functionalized to support the attachment and synthesis of a library of polynucleotides. The device surface was first wet cleaned using a piranha solution comprising 90% H₂SO₄ and 10% H₂O₂ for 20 minutes. The device was rinsed in several beakers with DI water, held under a DI water gooseneck faucet for 5 min, and dried with N₂. The device was subsequently soaked in NH₄OH (1:100; 3 mL:300 mL) for 5 min, rinsed with DI water using a handgun, soaked in three successive beakers with DI water for 1 min each, and then rinsed again with DI water using the handgun. The device was then plasma cleaned by exposing the device surface to O₂. A SAMCO PC-300 instrument was used to plasma etch O₂ at 250 watts for 1 min in downstream mode.

The cleaned device surface was actively functionalized with a solution comprising N-(3-triethoxysilylpropyl)-4-hydroxybutyramide using a YES-1224P vapor deposition oven system with the following parameters: 0.5 to 1 torr, 60 min, 70° C., 135° C. vaporizer. The device surface was resist coated using a Brewer Science 200X spin coater. SPR™ 3612 photoresist was spin coated on the device at 2500 rpm for 40 sec. The device was pre-baked for 30 min at 90° C. on a Brewer hot plate. The device was subjected to photolithography using a Karl Suss MA6 mask aligner instrument. The device was exposed for 2.2 sec and developed for 1 min in MSF 26A. Remaining developer was rinsed with the handgun and the device soaked in water for 5 min. The device was baked for 30 min at 100° C. in the oven, followed by visual inspection for lithography defects using a Nikon L200. A cleaning process was used to remove residual resist using the SAMCO PC-300 instrument to O₂ plasma etch at 250 watts for 1 min.

The device surface was passively functionalized with a 100 μL solution of perfluorooctyltrichlorosilane mixed with 10 μL light mineral oil. The device was placed in a chamber, pumped for 10 min, and then the valve was closed to the pump and left to stand for 10 min. The chamber was vented to air. The device was resist stripped by performing two soaks for 5 min in 500 mL NMP at 70° C. with ultrasonication at maximum power (9 on Crest system). The device was then soaked for 5 min in 500 mL isopropanol at room temperature with ultrasonication at maximum power. The device was dipped in 300 mL of 200 proof ethanol and blown dry with N₂. The functionalized surface was activated to serve as a support for polynucleotide synthesis.

### Example 2: Synthesis of a 50-Mer Sequence on a Polynucleotide Synthesis Device

A two dimensional polynucleotide synthesis device was assembled into a flowcell, which was connected to a flowcell (Applied Biosystems (ABI394 DNA Synthesizer″). The polynucleotide synthesis device was uniformly functionalized with N-(3-TRIETHOXYSILYLPROPYL)-4-HYDROXYBUTYRAMIDE (Gelest) was used to synthesize an exemplary polynucleotide of 50 bp (“50-mer polynucleotide”) using polynucleotide synthesis methods described herein.

The sequence of the 50-mer was as described in SEQ ID NO.: 1. 5′AGACAATCAACCATTTGGGGTGGACAGCCTTGACCTCTAGACTTCGGCAT ##TTTTTTTTT T3′ (SEQ ID NO.: 1), where #denotes Thymidine-succinyl hexamide CED phosphoramidite (CLP-2244 from ChemGenes), which is a cleavable linker enabling the release of polynucleotides from the surface during deprotection.

The synthesis was done using standard DNA synthesis chemistry (coupling, capping, oxidation, and deblocking) according to the protocol in Table 2 and an ABI synthesizer.

TABLE 2

| General DNA Synthesis | | |
|---|---|---|
| Process Name | Process Step | Time (sec) |
| WASH (Acetonitrile | Acetonitrile System Flush | 4 |
| Wash Flow) | Acetonitrile to Flowcell | 23 |
| | N2 System Flush | 4 |
| | Acetonitrile System Flush | 4 |
| DNA BASE ADDITION | Activator Manifold Flush | 2 |
| (Phosphoramidite + | Activator to Flowcell | 6 |
| Activator Flow) | Activator + | 6 |
| | Phosphoramidite to | |
| | Flowcell | |
| | Activator to Flowcell | 0.5 |
| | Activator + | 5 |
| | Phosphoramidite to | |
| | Flowcell | |
| | Activator to Flowcell | 0.5 |
| | Activator + | 5 |
| | Phosphoramidite to | |
| | Flowcell | |
| | Activator to Flowcell | 0.5 |
| | Activator + | 5 |
| | Phosphoramidite to | |
| | Flowcell | |
| | Incubate for 25 sec | 25 |
| WASH (Acetonitrile | Acetonitrile System Flush | 4 |
| Wash Flow) | Acetonitrile to Flowcell | 15 |
| | N2 System Flush | 4 |
| | Acetonitrile System Flush | 4 |
| DNA BASE ADDITION | Activator Manifold Flush | 2 |
| (Phosphoramidite + | Activator to Flowcell | 5 |
| Activator Flow) | Activator + | 18 |
| | Phosphoramidite to | |
| | Flowcell | |
| | Incubate for 25 sec | 25 |
| WASH (Acetonitrile | Acetonitrile System Flush | 4 |
| Wash Flow) | Acetonitrile to Flowcell | 15 |
| | N2 System Flush | 4 |
| | Acetonitrile System Flush | 4 |
| CAPPING (CapA + | CapA + B to Flowcell | 15 |
| B, 1:1, Flow) | | |
| WASH (Acetonitrile | Acetonitrile System Flush | 4 |
| Wash Flow) | Acetonitrile to Flowcell | 15 |
| | Acetonitrile System Flush | 4 |
| OXIDATION (Oxidizer | Oxidizer to Flowcell | 18 |
| Flow) | | |
| WASH (Acetonitrile | Acetonitrile System Flush | 4 |
| Wash Flow) | N2 System Flush | 4 |
| | Acetonitrile System Flush | 4 |
| | Acetonitrile to Flowcell | 15 |
| | Acetonitrile System Flush | 4 |
| | Acetonitrile to Flowcell | 15 |
| | N2 System Flush | 4 |
| | Acetonitrile System Flush | 4 |
| | Acetonitrile to Flowcell | 23 |
| | N2 System Flush | 4 |
| | Acetonitrile System Flush | 4 |
| DEBLOCKING | Deblock to Flowcell | 36 |
| (Deblock Flow) | | |
| WASH (Acetonitrile | Acetonitrile System Flush | 4 |
| Wash Flow) | N2 System Flush | 4 |
| | Acetonitrile System Flush | 4 |
| | Acetonitrile to Flowcell | 18 |
| | N2 System Flush | 4.13 |
| | Acetonitrile System Flush | 4.13 |
| | Acetonitrile to Flowcell | 15 |

The phosphoramidite/activator combination was delivered similar to the delivery of bulk reagents through the flowcell. No drying steps were performed as the environment stays “wet” with reagent the entire time.

The flow restrictor was removed from the ABI 394 synthesizer to enable faster flow. Without flow restrictor, flow rates for amidites (0.1 M in ACN), Activator, (0.25M Benzoylthiotetrazole (“BTT”; 30-3070-xx from GlenResearch) in ACN), and Ox (0.02M 12 in 20% pyridine, 10% water, and 70% THF) were roughly ~200 uL/sec, for acetonitrile (“ACN”) and capping reagents (1:1 mix of CapA and CapB, wherein CapA is acetic anhydride in THF/Pyridine and CapB is 16% 1-methylimidizole in THF), roughly ~200 uL/sec, and for Deblock (3% dichloroacetic acid in toluene), roughly ~300 uL/sec (compared to ~50 uL/sec for all reagents with flow restrictor). The time to completely push out Oxidizer was observed, the timing for chemical flow times was adjusted accordingly and an extra ACN wash was introduced between different chemicals. After polynucleotide synthesis, the chip was deprotected in gaseous ammonia overnight at 75 psi. Five drops of water were applied to the surface to recover polynucleotides. The recovered polynucleotides were then analyzed on a BioAnalyzer small RNA chip (data not shown).

### Example 3: Voltage Optimization

Cyclic voltammetry was used to evaluate redox potential for pairs of compounds shown in Table 3.

TABLE 3
Redox Pairs

| Pair | Compounds | |
|---|---|---|
| E1 | 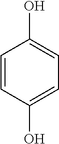 | 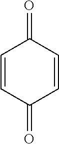 |
| E2 | 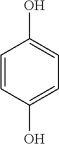 | 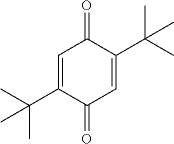 [SMILES: CC(C)(C)C1=CC(=O)C(C(C)(C)C)=CC1=O] |
| E3 | 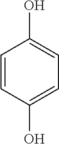 | 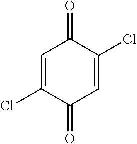 |
| E4 | 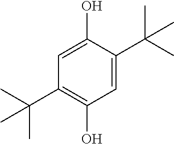 [SMILES: CC(C)(C)c1cc(O)c(C(C)(C)C)cc1O] | 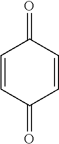 |
| E5 | 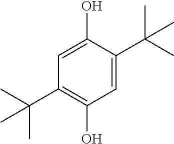 [SMILES: CC(C)(C)c1cc(O)c(C(C)(C)C)cc1O] | 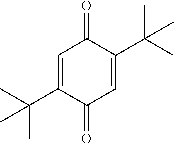 [SMILES: CC(C)(C)C1=CC(=O)C(C(C)(C)C)=CC1=O] |
| E6 | 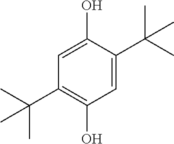 [SMILES: CC(C)(C)c1cc(O)c(C(C)(C)C)cc1O] | 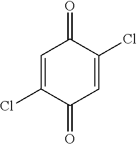 |
| E7 | 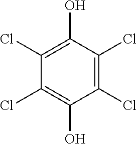 [SMILES: Oc1c(Cl)c(Cl)c(O)c(Cl)c1Cl] | 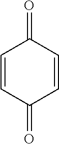 |
| E8 | 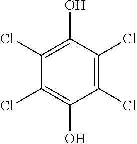 [SMILES: Oc1c(Cl)c(Cl)c(O)c(Cl)c1Cl] | 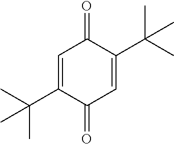 [SMILES: CC(C)(C)C1=CC(=O)C(C(C)(C)C)=CC1=O] |
| E9 | 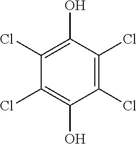 [SMILES: Oc1c(Cl)c(Cl)c(O)c(Cl)c1Cl] | 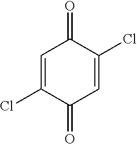 |
| E10 | 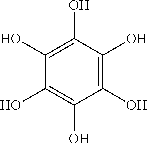 [SMILES: Oc1c(O)c(O)c(O)c(O)c1O] | 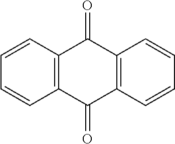 [SMILES: O=C1c2ccccc2C(=O)c2ccccc21] |
| E11 | 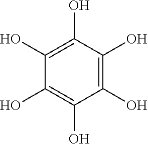 [SMILES: Oc1c(O)c(O)c(O)c(O)c1O] | 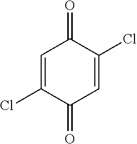 |
| E12 | 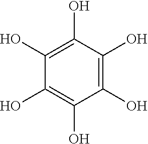 [SMILES: Oc1c(O)c(O)c(O)c(O)c1O] | 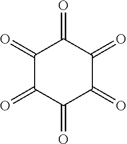 [SMILES: O=C1C(=O)C(=O)C(=O)C(=O)C1=O] |
| E13 | 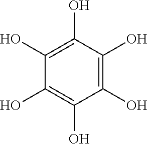 [SMILES: Oc1c(O)c(O)c(O)c(O)c1O] | 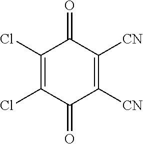 [SMILES: N#CC1=C(C#N)C(=O)C(Cl)=C(Cl)C1=O] |
| E14 | 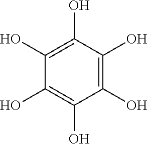 [SMILES: Oc1c(O)c(O)c(O)c(O)c1O] | 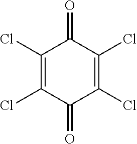 [SMILES: O=C1C(Cl)=C(Cl)C(=O)C(Cl)=C1Cl] |
| E15 | 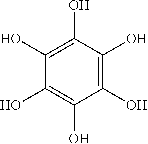 [SMILES: Oc1c(O)c(O)c(O)c(O)c1O] | 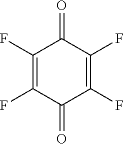 [SMILES: O=C1C(F)=C(F)C(=O)C(F)=C1F] |
| E16 | 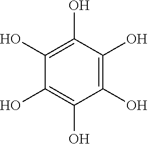 [SMILES: Oc1c(O)c(O)c(O)c(O)c1O] | 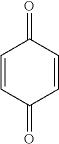 |
| E17 | 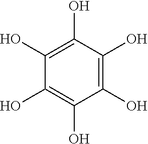 [SMILES: Oc1c(O)c(O)c(O)c(O)c1O] | 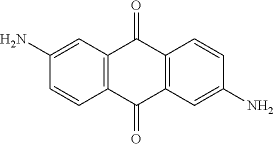 [SMILES: Nc1ccc2c(c1)C(=O)c1ccc(N)cc1C2=O] |
| E18 | 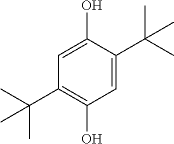 [SMILES: CC(C)(C)c1cc(O)c(C(C)(C)C)cc1O] | 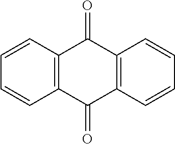 [SMILES: O=C1c2ccccc2C(=O)c2ccccc21] |
| E19 | 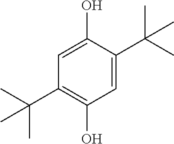 [SMILES: CC(C)(C)c1cc(O)c(C(C)(C)C)cc1O] | 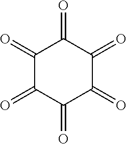 [SMILES: O=C1C(=O)C(=O)C(=O)C(=O)C1=O] |
| E20 | 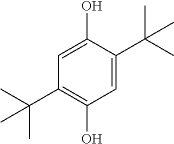 [SMILES: CC(C)(C)c1cc(O)c(C(C)(C)C)cc1O] | 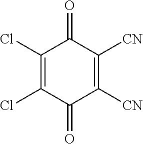 [SMILES: N#CC1=C(C#N)C(=O)C(Cl)=C(Cl)C1=O] |
| E21 | 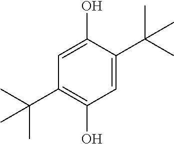 [SMILES: CC(C)(C)c1cc(O)c(C(C)(C)C)cc1O] | 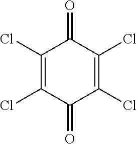 [SMILES: O=C1C(Cl)=C(Cl)C(=O)C(Cl)=C1Cl] |
| E22 | 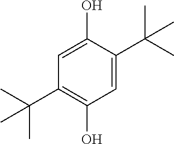 [SMILES: CC(C)(C)c1cc(O)c(C(C)(C)C)cc1O] | 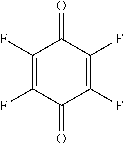 [SMILES: O=C1C(F)=C(F)C(=O)C(F)=C1F] |
| E23 | 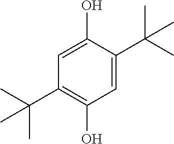 [SMILES: CC(C)(C)c1cc(O)c(C(C)(C)C)cc1O] | 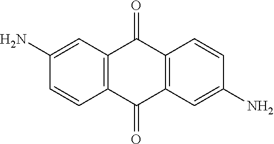 [SMILES: Nc1ccc2c(c1)C(=O)c1ccc(N)cc1C2=O] |
| E24 | 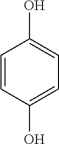 | 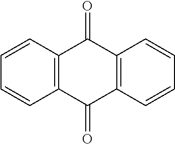 [SMILES: O=C1c2ccccc2C(=O)c2ccccc21] |
| E25 | 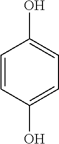 | 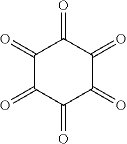 [SMILES: O=C1C(=O)C(=O)C(=O)C(=O)C1=O] |
| E26 | 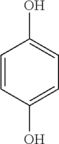 | 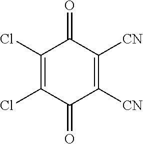 [SMILES: N#CC1=C(C#N)C(=O)C(Cl)=C(Cl)C1=O] |
| E27 | 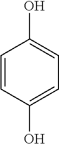 | 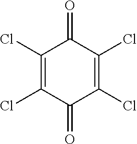 [SMILES: O=C1C(Cl)=C(Cl)C(=O)C(Cl)=C1Cl] |
| E28 | 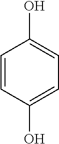 | 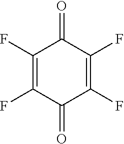 [SMILES: O=C1C(F)=C(F)C(=O)C(F)=C1F] |
| E29 | 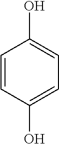 | 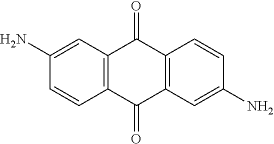 [SMILES: Nc1ccc2c(c1)C(=O)c1ccc(N)cc1C2=O] |
| E30 | 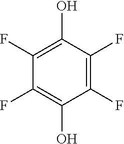 [SMILES: Oc1c(F)c(F)c(O)c(F)c1F] | 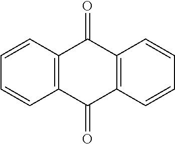 [SMILES: O=C1c2ccccc2C(=O)c2ccccc21] |
| E31 | 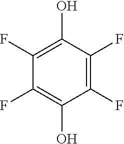 [SMILES: Oc1c(F)c(F)c(O)c(F)c1F] | 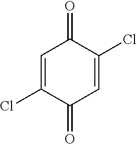 |
| E32 | 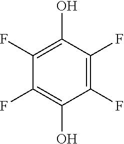 [SMILES: Oc1c(F)c(F)c(O)c(F)c1F] | 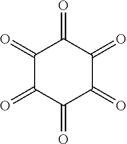 [SMILES: O=C1C(=O)C(=O)C(=O)C(=O)C1=O] |
| E33 | 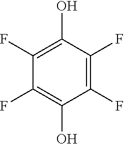 [SMILES: Oc1c(F)c(F)c(O)c(F)c1F] | 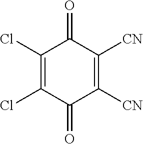 [SMILES: N#CC1=C(C#N)C(=O)C(Cl)=C(Cl)C1=O] |
| E34 | 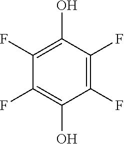 [SMILES: Oc1c(F)c(F)c(O)c(F)c1F] | 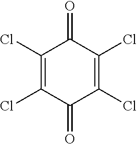 [SMILES: O=C1C(Cl)=C(Cl)C(=O)C(Cl)=C1Cl] |
| E35 | 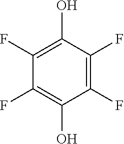 [SMILES: Oc1c(F)c(F)c(O)c(F)c1F] | 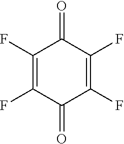 [SMILES: O=C1C(F)=C(F)C(=O)C(F)=C1F] |
| E36 | 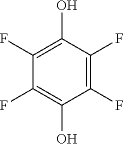 [SMILES: Oc1c(F)c(F)c(O)c(F)c1F] | 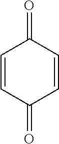 |
| E37 | 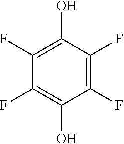 [SMILES: Oc1c(F)c(F)c(O)c(F)c1F] | 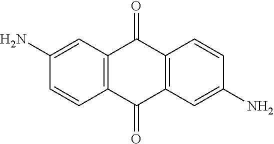 [SMILES: Nc1ccc2c(c1)C(=O)c1ccc(N)cc1C2=O] |
| E38 | 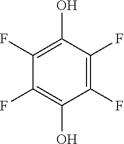 [SMILES: Oc1c(F)c(F)c(O)c(F)c1F] | 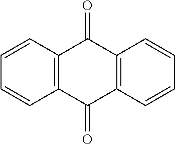 [SMILES: O=C1c2ccccc2C(=O)c2ccccc21] |
| E39 | 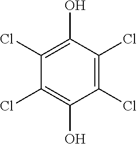 [SMILES: Oc1c(Cl)c(Cl)c(O)c(Cl)c1Cl] | 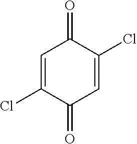 |
| E40 | 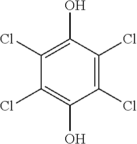 [SMILES: Oc1c(Cl)c(Cl)c(O)c(Cl)c1Cl] | 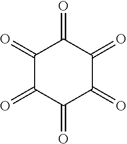 [SMILES: O=C1C(=O)C(=O)C(=O)C(=O)C1=O] |
| E41 | 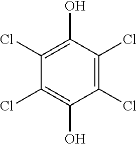 [SMILES: Oc1c(Cl)c(Cl)c(O)c(Cl)c1Cl] | 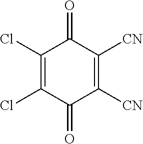 [SMILES: N#CC1=C(C#N)C(=O)C(Cl)=C(Cl)C1=O] |
| E42 | 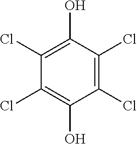 [SMILES: Oc1c(Cl)c(Cl)c(O)c(Cl)c1Cl] | 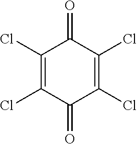 [SMILES: O=C1C(Cl)=C(Cl)C(=O)C(Cl)=C1Cl] |
| E43 | 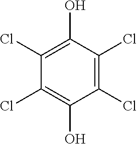 [SMILES: Oc1c(Cl)c(Cl)c(O)c(Cl)c1Cl] | 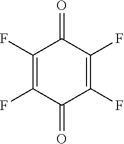 [SMILES: O=C1C(F)=C(F)C(=O)C(F)=C1F] |

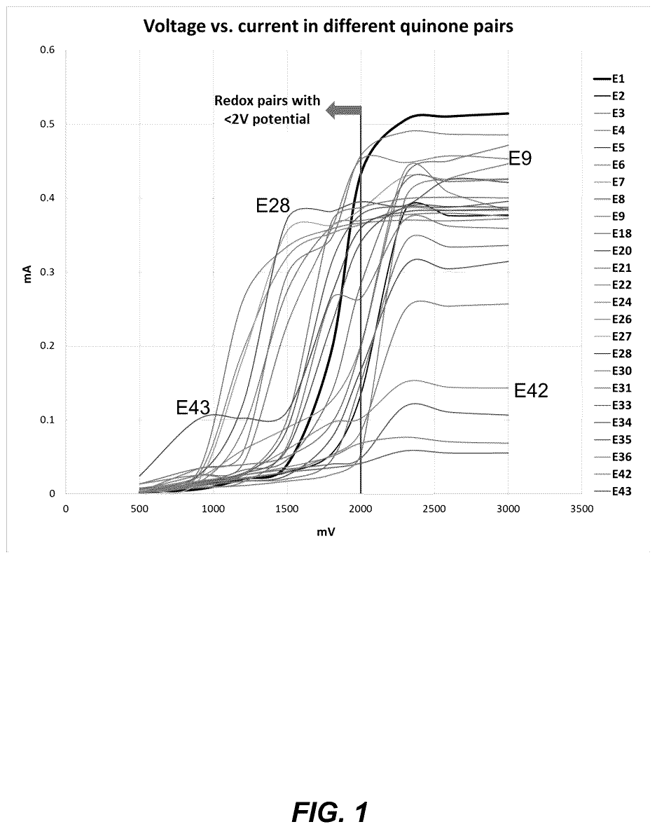

Oxidation potentials were measured from the current traces generated (FIG. 1) across a range of 0.5 to 3 volts. Compound pairs E9, E28, E42, and E43 showed redox potentials lower than 2 volts.

### Example 3: Electrochemical Deblocking During Polynucleotide Synthesis

A device comprising the layout of FIG. 2E was synthesized using general platinum patterning methods and synthesis surface preparation methods of Example 1. Each square represents an individually addressable loci for polynucleotide synthesis. The device had a pitch of 10 microns, with a 5-mer thymine stilt attached to the loci. Polynucleotide synthesis steps were conducted for 15 cycles, following the general methods of Examples 1 and 2 with modification; the chemical deblocking step was replaced with an electrochemical deblocking step comprising treatment of the surface with compound combination E42 (each at a concentration of 1 mM), 17 mM tetrabutylammonium hexafluorophosphate (17 mM) and application of a voltage (1.9V). The deblocking step was carried out with a voltage cycle of 1 second on, 1 second off. Three voltage cycles were used per deblocking step. Acetonitrile was used as the deblocking solvent.

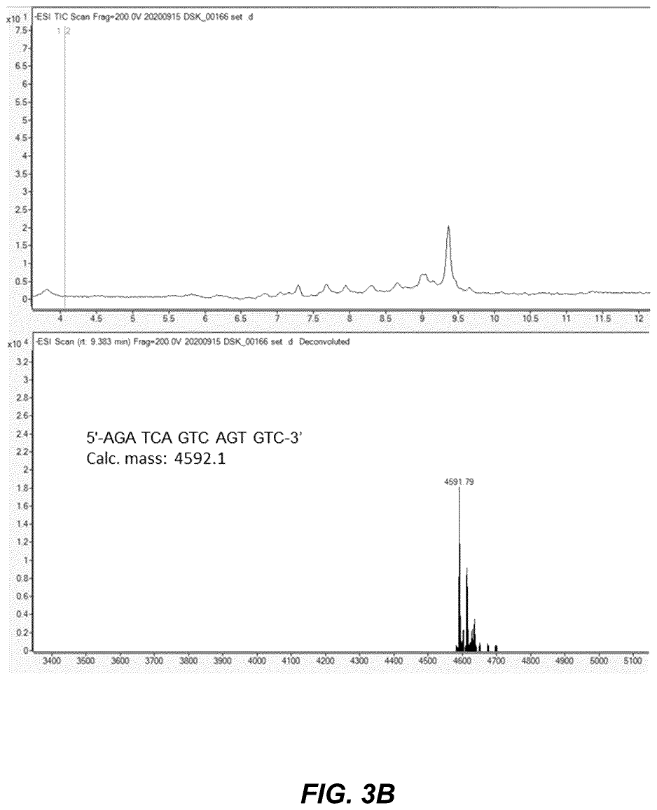

Prior to cleavage of the polynucleotide products, a fluorescently labeled nucleotide, TAMRA labeled dT (5′-dimethoxytrityloxy-5-[N-((tetramethylrhodaminyl)-aminohexyl)-3-acrylimido]-2′-deoxyuridine-3′-[(2-cyanoethyl)-(N,N-diisopropyl)]-phosphoramidite), is added to selectively deprotected polynucleotides. Lighter areas indicate the presence of the labeled dT incorporated into a surface-bound polynucleotide (FIG. 3A). After synthesis, the mixed-base 15-mer (AGATCAGTCAGTGTC (SEQ ID NO: 2)) was cleaved from the surface and analyzed by LCMS (FIG. 3B).

### Example 4: Data Storage

A device of Example 4 is used following the general procedures of Example 3 to synthesize an array comprising a plurality of polynucleotides, where the plurality of polynucleotides collectively encode for digital information. The polynucleotides are optionally cleaved from the device surface and sequenced, or sequenced directly from the device. The sequences obtained are then converted into the digital information.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-36. (canceled)

37. A method for polynucleotide synthesis comprising:
contacting a nucleoside attached to a solid support with a protected nucleoside, wherein the protected nucleoside is configured to form a covalent bond with the nucleoside to generate a protected polynucleotide comprising an acid cleavable protecting group;
contacting the protected polynucleotide with a composition comprising a first redox compound, and a second redox compound, wherein a redox potential between the first redox compound and the second redox compound is less than 2 volts; and
applying, in a series of pulses, a voltage to a solvent in fluid communication with the protected polynucleotide, wherein the voltage results in deprotection of a terminal nucleoside of the protected polynucleotide.

38. The method of claim 37, wherein the voltage is less than 2 volts.

39. The method of claim 37, wherein the voltage is applied in a series of 10-5,000 pulses.

40. The method of claim 37, wherein a total amount of time the voltage is applied is about 0.0001 to about 0.5 seconds.

41. The method of claim 37, wherein a total amount of time the voltage is applied is about 0.1 to about 10 seconds.

42. The method of claim 37, wherein a voltage application time per pulse is about 0.1 to about 10 microseconds.

43. The method of claim 37, wherein voltage applied in a first pulse is different from voltage applied in a second pulse.

44. The method of claim 39, wherein a ratio between voltage on and voltage off time for the series of pulses is less than 3 to 1.

45. The method of claim 37, wherein the composition further comprises at least one solvent, and wherein the at least one solvent is acetonitrile, methanol, ethanol, dichloromethane, chloroform, 1,2-dichloromethane, dimethylformamide, ethylene glycol, propylene carbonate, or a mixture thereof.

46. The method of claim 37, wherein the composition further comprises a salt, wherein the salt comprises hexafluorophosphate, tetrafluoroborate, tetraphenylborate, hexafluorophosphate, perchlorate, tetrachloroferrate, hexafluoroarsenate, hexafluoroantimonate, pentafluorohy-droxyantimonate, hexachloroantimonate, tetrakispentafluorophenylborate, tetrakis-(pentafluoromethylphenyl) borate, bi(trifluoromethylsulphonyl)amides, tris(trifluoromethylsulphonyl)methides, or tetrabutylammonium hexafluorophosphate, and wherein the salt is present at a concentration of about 10 to about 50 mM.

47. The method of claim 37, further comprising repeating steps of claim 37 to synthesize polynucleotides that are 25-500 bases long, and wherein the synthesized polynucleotides have an error rate of less than about 1 in 5,000 bases.

48. The method of claim 37, wherein the first redox compound or the second redox compound independently has a structure: 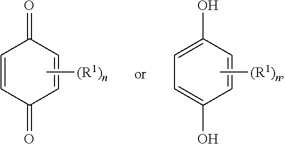
wherein:
each R1 is independently hydrogen, halogen, —CN, —ORa, —SRa, —S(═O)Rb, —NO₂, —NRcRd, —S(═O)₂Rd, —NRaS(═O)₂Rd, —S(═O)₂NRcRd, —C(═O)Rb, —OC(═O)Rb, —CO₂Ra, —OCO₂Ra, —C(═O)NRcRd, —OC(═O)NRcRd, —NRaC(═O)NRcRd, —NRaC(═O)Rb, —NRaC(═O)ORa, C1-C6 alkyl, C2-C6 alkenyl, C2-C6 alkynyl, C1-C6 heteroalkyl, C3-C8 cycloalkyl, C2-C8 heterocycloalkyl, aryl, or heteroaryl; or two or more R1 are taken together to form a cycloalkyl, heterocycloalkyl, aryl, or heteroaryl ring; and
wherein:
Ra is hydrogen, C1-C6 alkyl, C2-C6 alkenyl, C2-C6 alkynyl, C1-C6 heteroalkyl, C3-C8 cycloalkyl, C2-C8 heterocycloalkyl, aryl, or heteroaryl;
Rb is C1-C6 alkyl, C2-C6 alkenyl, C2-C6 alkynyl, C1-C6 heteroalkyl, C3-C8 cycloalkyl, C2-C8 heterocycloalkyl, aryl, or heteroaryl;
each Rc and Rd is independently hydrogen, C1-C6 alkyl, C2-C6 alkenyl, C2-C6 alkynyl, C1-C6 heteroalkyl, C3-C8 cycloalkyl, C2-C8 heterocycloalkyl, aryl, or heteroaryl; or
Rc and Rd, together with each nitrogen atom to which they are attached, form a heterocycloalkyl or heteroaryl; and
n is 0-4.

49. The method of claim 48, wherein the first redox compound has the structure: 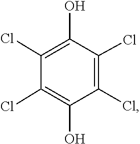 [SMILES: Oc1c(Cl)c(Cl)c(O)c(Cl)c1Cl]
nd the second redox compound has the structure

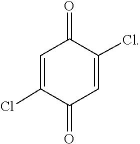

50. The method of claim 48, wherein the first redox compound has a structure: 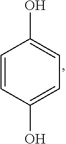
and the second redox compound has the structure

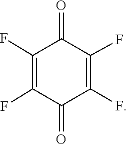 [SMILES: O=C1C(F)=C(F)C(=O)C(F)=C1F]

51. The method of claim 48, wherein the first redox compound has the structure: 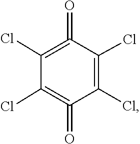
and the second redox compound has the structure

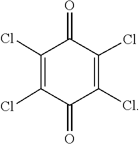 [SMILES: O=C1C(Cl)=C(Cl)C(=O)C(Cl)=C1Cl]

52. The method of claim 48, wherein the first redox compound has the structure: 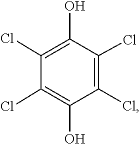 [SMILES: Oc1c(Cl)c(Cl)c(O)c(Cl)c1Cl]
and the second redox compound has the structure

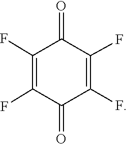 [SMILES: O=C1C(F)=C(F)C(=O)C(F)=C1F]

53. The method of claim 37, wherein a concentration of the first redox compound and a concentration of the second redox compound is about 0.1 to about 2M.

54. The method of claim 37, wherein the composition does not comprise a base.

55. The method of claim 37, wherein the solid support comprises:
a surface comprising a plurality of loci configured for polynucleotide synthesis, wherein the loci comprises a pitch distance of less than 1,000 nm; and
a plurality of vias and/or routing configured for addressable control of the plurality of loci.

56. The method of claim 55, further comprising washing the surface of the solid support in between pulses.
